(19) United States
(12) Patent Application Publication
(10) Pub. No.: US 2025/0171390 A1
(43) Pub. Date: May 29, 2025

(54) PROCESS FOR PREPARING UNSATURATED ALDEHYDES

(71) Applicant: Firmenich SA, Satigny (CH)

(72) Inventors: Oliver KNOPFF, Satigny (CH); Jean-Jacques RIEDHAUSER, Satigny (CH); Nicolas POIRIER, Satigny (CH); Luigi MARINONI, Satigny (CH)

(21) Appl. No.: 18/839,565

(22) PCT Filed: Mar. 1, 2023
(86) PCT No.: PCT/EP2023/055070

§ 371 Date: Aug. 19, 2024

(30) Foreign Application Priority Data
Mar. 1, 2022 (EP) 22159462.5

(51) Int. Cl. C07C 41/48; C07C 29/38

(52) U.S. Cl. CPC C07C 41/48; C07C 29/38; C07C 2523/46; C07C 2601/16

(57) ABSTRACT

Disclosed herein is a process for preparing a compound of formula (I) starting from a compound of formula (II). Also disclosed herein are a process to prepare a compound of formula (II) and the compound of formula (II).

### TECHNICAL FIELD

The present invention relates to the field of organic synthesis and more specifically it concerns a process for preparing compound of formula (I) starting from a compound of formula (II). The process to prepare compound of formula (II) and the compound of formula (II) are also part of the invention.

### BACKGROUND OF THE INVENTION

The access to compounds comprising unsaturated aldehydes, such as beta,gamma-, gamma,delta- or delta,epsilon-unsaturated aldehydes, is highly sought as they represent highly desirable skeletons which could be used as such or as key intermediates useful to prepare more complex compounds in different fields such as, among others, perfumery, cosmetic, pharmaceutic or agrochemistry. A multitude of methodologies has been developed in this context. However, said methodologies suffer from low selectivity, low yield and/or request several steps.

Being products of industrial interest, there is always a need for new processes showing an improved yield and selectivity.

So, there is a need to develop a straightforward methodology to access such compounds with high yield and selectivity.

The present invention provides a solution to the above problem by treating a tetrahydrofuran-2-ol or tetrahydro-2H-pyran-2-ol derivative, obtained via hydroformylation, with an acid in the presence of a polyol or an alpha hydroxy carboxylic acid. To the best of our knowledge, in the prior art there is no report of such a rearrangement as disclosed in the present invention.

### SUMMARY OF THE INVENTION

The invention relates to a novel process allowing the preparation of compound of formula (I) with a high yield and high selectivity starting from compound of formula (II). The invention process represents a new efficient route toward compound of formula (I) which could be easily converted into the corresponding aldehyde of formula (V).

So, the first object of the present invention is a process for the preparation of a compound of formula

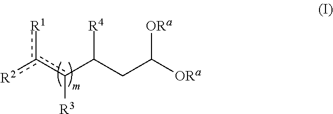

in the form of any one of its stereoisomers or a mixture thereof, and wherein one dotted line is a carbon-carbon double bond and the others are carbon-carbon single bonds; m is 0 or 1; R¹ represents a C₁₋₁₂ hydrocarbon group, optionally comprising one to three oxygen atoms and/or one to two nitrogen atoms and/or one sulfur atom; R² represents a hydrogen atom or a R¹ group; or R¹ and R² are taken together and form a C₅₋₁₈ cycloalkyl or C₅₋₁₈ cycloalkenyl group, each optionally substituted by one or more hydroxy, C₁₋₁₅ alkyl, C₂₋₁₅ alkenyl, C₁₋₁₅ alkoxy, C₃₋₁₅ cycloalkyl, C₅₋₁₅ cycloalkenyl, C₁₋₆ ester, COOH, C₆₋₁₀ aryl and/or C₆₋₁₀ aryloxy groups; R³ and R⁴, independently from each other, represent a hydrogen atom or a C₁₋₆ alkyl group; both R^{a} are taken together and represent a C₂₋₂₀ hydrocarbylene group, optionally comprising one to five oxygen atoms;
comprising the reaction of a compound of formula (II)

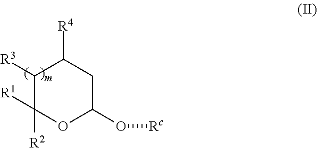

in the form of any one of its stereoisomers or a mixture thereof, and wherein R¹, R², R³, R⁴ and m have the same meaning as defined in formula (I) and R^{c} is a hydrogen atom, a C₁₋₁₂ hydrocarbon group optionally comprising one to five oxygen atoms or R^{c} is a group of formula

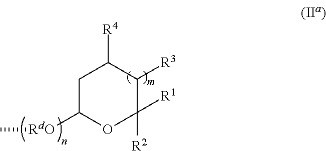

wherein R¹, R², R³, R⁴ and m have the same meaning as defined in formula (I); n is 0 or 1, R^{d} is a C₁₋₁₂ hydrocarbylene group optionally comprising one to five oxygen atoms and the hatched line indicates the location of the bond between (II^{a}) and the oxygen atom of formula (II);
with an acid and a polyol or an alpha hydroxy carboxylic acid.

A second object of the present invention is a process for the preparation of a compound of formula

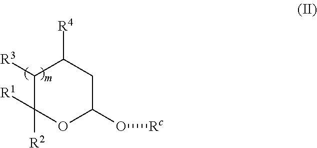

in the form of any one of its stereoisomers or a mixture thereof, and wherein m is 0 or 1; R¹ represents a C₁₋₁₂ hydrocarbon group, optionally comprising one to three oxygen atoms and/or one to two nitrogen atoms and/or one sulfur atom; R² represents a hydrogen atom or a R¹ group; or R¹ and R² are taken together and form C₅₋₁₈ cycloalkyl or C₅₋₁₈ cycloalkenyl group, each optionally substituted by one or more hydroxy, C₁₋₁₅ alkyl, C₂₋₁₅ alkenyl, C₁₋₁₅ alkoxy, C₃₋₁₅ cycloalkyl, C₅₋₁₅ cycloalkenyl, C₁₋₆ ester, COOH, C₆₋₁₀ aryl and/or C₆₋₁₀ aryloxy groups; R³ and R⁴, independently from each other, represent a hydrogen atom or a C₁₋₆ alkyl group; and R^{c} is a hydrogen atom, a C₁₋₁₂ hydrocarbon group optionally comprising one to five oxygen atoms or a R^{c} is a group of formula

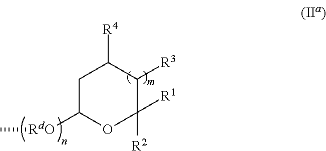

wherein R¹, R², R³, R⁴ and m have the same meaning as defined in formula (II); n is 0 or 1, R^{d} is a C₁₁₋₁₂ hydrocarbylene group optionally comprising one to five oxygen atoms and the hatched line indicates the location of the bond between (II^{a}) and the oxygen atom of formula (II);
by the hydroformylation of a compound of formula

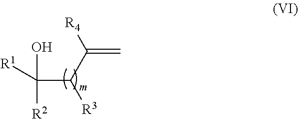

in the form of any one of its stereoisomers or a mixture thereof, and wherein m, R¹, R², R³ and R⁴ have the same meaning as defined in formula (II);
in a presence of a hydroformylation catalyst comprising a bidentate phosphorous ligand and optionally an alcohol or a polyol.

A further object of the invention is a compound of formula

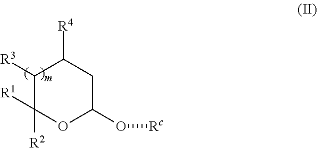

in the form of any one of its stereoisomers or a mixture thereof, and wherein m is 0 or 1; R¹ and R², independently from each other, represent a C₁₋₁₂ hydrocarbon group, optionally comprising one to three oxygen atoms and/or one to two nitrogen atoms and/or one sulfur atom; or R¹ and R² are taken together and form C₅₋₁₈ cycloalkyl or C₅₋₁₈ cycloalkenyl group, each optionally substituted by one or more hydroxy, C₁₋₁₅ alkyl, C₂₋₁₅ alkenyl, C₁₋₁₅ alkoxy, C₃₋₁₅ cycloalkyl, C₅₋₁₅ cycloalkenyl, C₁₋₆ ester, COOH, C₆₋₁₀ aryl and/or C₆₋₁₀ aryloxy groups; R³ and R⁴, independently from each other, represent a hydrogen atom or a C₁₋₆ alkyl group; and R^{c} is a hydrogen atom, a C₁₋₁₂ hydrocarbon group optionally comprising one to five oxygen atoms or a R^{c} is a group of formula

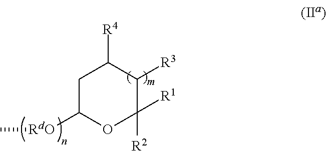

wherein R¹, R², R³, R⁴ and m have the same meaning as defined in formula (II); n is 0 or 1, R^{d} is a C₁₋₁₂ hydrocarbylene group optionally comprising one to five oxygen atoms and the hatched line indicates the location of the bond between (II^{a}) and the oxygen atom of formula (H); provided that 4-butyl-1-[(5,5-dibutyltetrahydro-2-furayl)oxy]-2,4-octanediol and 2-[(Tetrahydro-5,5-dimethyl-2-furanyl)oxy]ethanol are excluded.

### DESCRIPTION OF THE INVENTION

It has now been surprisingly found that the compound of formula (H), in a presence of acid and a polyol, in particular a diol, a triol, a tetraol, or an alpha hydroxy carboxylic acid provides a non-conjugated-unsaturated aldehyde with high yield and selectivity. Said rearrangement is novel and has never been reported. Moreover, the compound of formula (H) may be easily obtained via the hydroformylation of a allylic or homoallylic alcohol The present inventions provide a three-step straightforward approach toward non-conjugated unsaturated aldehydes.

So, the first object of the invention is a process for the preparation of a compound of formula

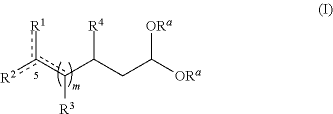

in the form of any one of its stereoisomers or a mixture thereof, and wherein one dotted line is a carbon-carbon double bond and the others are carbon-carbon single bonds; m is 0 or 1; R¹ represents a C₁₋₁₂ hydrocarbon group, optionally comprising one to three oxygen atoms and/or one to two nitrogen atoms and/or one sulfur atom; R² represents a hydrogen atom or a R¹ group; or R¹ and R² are taken together and form a C₅₋₁₈ cycloalkyl or C₅₋₁₈ cycloalkenyl group, each optionally substituted by one or more hydroxy, C₁₋₁₅ alkyl, C₂₋₁₅ alkenyl, C₁₋₁₅ alkoxy, C₃₋₁₅ cycloalkyl, C₅₋₁₅ cycloalkenyl, C₁₋₆ ester, COOH, C₆₋₁₀ aryl and/or C₆₋₁₀ aryloxy groups; R³ and R⁴, independently from each other, represent a hydrogen atom or a C₁₋₆ alkyl group; both R^{a} are taken together and represent a C₂₋₂₀ hydrocarbylene group, optionally comprising one to five oxygen atoms;
comprising the reaction of a compound of formula (H)

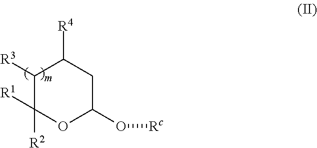

in the form of any one of its stereoisomers or a mixture thereof, and wherein R¹, R², R³, R⁴ and m have the same meaning as defined in formula (I) and R^{c} is a hydrogen atom, a C₁₋₁₂ hydrocarbon group optionally comprising one to five oxygen atoms or R^{c} is a group of formula

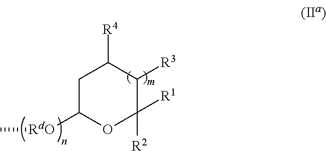

wherein R¹, R², R³, R⁴ and m have the same meaning as defined in formula (I); n is 0 or 1, R^{d} is a C₁₋₁₂ hydrocarbylene group optionally comprising one to five oxygen atoms and the hatched line indicates the location of the bond between (II^{a}) and the oxygen atom of formula (II);
with an acid and a polyol or an alpha hydroxy carboxylic acid.

For the sake of clarity, by the expression “any one of its stereoisomers or a mixture thereof”, or the similar, it is meant the normal meaning understood by a person skilled in the art, i.e. that the compound of formula (I) and (II) can be a pure enantiomer or a mixture of enantiomers. In other words, the compound of formula (I) and (II) may possess at least one stereocenter which can have two different stereochemistries (e.g. R or S). The compounds of formula (I) and (II) may even be in the form of a pure enantiomer or in the form of a mixture of enantiomers. The compounds of formula (I) and (II) may even be in the form of a pure diastereoisomer or in the form of a mixture of diastereoisomers when compounds of formula (I) and (II) possess more than one stereocenter. The compounds of formula (I) and (II) can be in a racemic form or scalemic form. Therefore, the compounds of formula (I) and (II) can be one stereoisomer or in the form of a composition of matter comprising, or consisting of, various stereoisomers.

For the sake of clarity, by the expression “one dotted line is a carbon-carbon double bond and the others are carbon-carbon single bonds”, or the similar, it is meant the normal meaning understood by a person skilled in the art, i.e. that the whole bonding (solid and dotted line) between the carbon atoms connected by said dotted line is a carbon-carbon single or double bond. The compound of formula (I) may be of formula

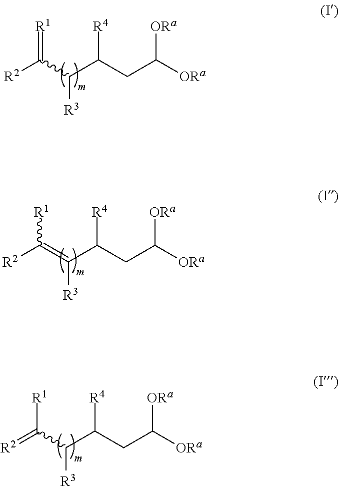

in the form of any one of its stereoisomers or a mixture thereof, and wherein R¹, R², R³, R⁴, R^{a} and m have the same meaning as defined above. The person skilled in the art is well aware that the compound of formula (I″′) is formed only when R² is not a hydrogen atom.

For the sake of clarity, by the wavy bond in compound of (I′), (I″) and (I″′), or the similar, it is meant the normal meaning understood by a person skilled in the art, i.e. that the double bond may have a cis configuration corresponding to the Z isomer, a trans configuration corresponding to the E isomer or a mixture thereof. Indeed, all compounds of the invention having a double bond may be in the form of its E or Z isomer or a mixture thereof. In other words, the compound of formula (I) may be in the form of its E or Z isomer or of a mixture thereof, e.g. the invention process leads to a composition of matter consisting of one or more compounds of formula (I), having the same chemical structure but differing by the configuration of the double bond. In particular, the compound of formula (I) can be in the form of a mixture consisting of isomers E and Z and wherein said isomer E represents at least 50% of the total mixture, or even at least 75% (i.e a mixture E/Z comprised between 75/25 and 100/0).

The term “optionally” is understood that a certain group to be optionally substituted can or cannot be substituted with a certain functional group. The term “one or more” is understood as being substituted with 1 to 7, preferably 1 to 5, preferably 1 to 3 and more preferably 1 to 2 of a certain functional group.

It is understood that by “ . . . hydrocarbon group . . . ” it is meant that said group consists of hydrogen and carbon atoms and can be in the form of an aliphatic hydrocarbon, i.e. linear or branched saturated hydrocarbon (e.g. alkyl group), a linear or branched unsaturated hydrocarbon (e.g. alkenyl or alkynyl group), a saturated cyclic hydrocarbon (e.g. cycloalkyl) or an unsaturated cyclic hydrocarbon (e.g. cycloalkenyl or cycloalkynyl), or can be in the form of an aromatic hydrocarbon, i.e. aryl group, or can also be in the form of a mixture of said type of groups, e.g. a specific group may comprise a linear alkyl, a branched alkenyl (e.g. having one or more carbon-carbon double bonds), a (poly)cycloalkyl and an aryl moiety, unless a specific limitation to only one type is mentioned. Similarly, in all the embodiments of the invention, when a group is mentioned as being in the form of more than one type of topology (e.g. linear, cyclic or branched) and/or being saturated or unsaturated (e.g. alkyl, aromatic or alkenyl), it is also meant a group which may comprise moieties having any one of said topologies or being saturated or unsaturated, as explained above. Similarly, in all the embodiments of the invention, when a group is mentioned as being in the form of one type of saturation or unsaturation, (e.g. alkyl), it is meant that said group can be in any type of topology (e.g. linear, cyclic or branched) or having several moieties with various topologies.

It is understood that with the term “ . . . a hydrocarbon group, optionally comprising . . . ” it is meant that said hydrocarbon group optionally comprises one two or three oxygen atoms in a form of alcohol, ketone, aldehyde, ether, ester, carboxylic acid, carbonate groups and/or one or two nitrogen atoms in a form of amine or amide groups and/or one sulfur atom in a form of a thiol group. These groups can either substitute a hydrogen atom of the hydrocarbon group and thus be laterally attached to said hydrocarbon, or substitute a carbon atom (if chemically possible) of the hydrocarbon group and thus be inserted into the hydrocarbon chain. For example, a —CH₂—CH₂—CHOH—CH₂— group represents a C₄ hydrocarbon group comprising an alcohol group (substitution of a hydrogen atom), i.e. a C₄ hydrocarbon comprising an oxygen atom; a —CH₂—CH₂—COO—CH₂—CH₂CH₂—CH₂— group represents a C₇ hydrocarbon group comprising one ester group (substitution of carbon atoms/insertion into the hydrocarbon chain), i.e. a C₇ hydrocarbon comprising two oxygen atoms and, similarly, a —CH₂—CH₂—O—CH₂—CH₂—O—CH₂—CH₂— group represents a C₆ hydrocarbon group comprising two ether groups, i.e. a C₆ hydrocarbon comprising two oxygen atoms. It is understood that the first atom of the R¹ or R² group connected to carbon 5 is always a carbon atom.

The terms “alkyl”, “alkenyl” and “alkoxy” are understood as comprising branched and linear alkyl and alkenyl groups. The terms “alkenyl” and “cycloalkenyl” are understood as comprising 1, 2 or 3 olefinic double bonds, preferably 1 or 2 olefinic double bonds. The terms “cycloalkyl” and “cycloalkenyl” are understood as comprising a monocyclic or fused, spiro and/or bridged bicyclic or tricyclic cycloalkyl and cycloalkenyl, groups, preferably monocyclic cycloalkyl and cycloalkenyl groups.

The term “aryl” are understood as comprising any group comprising at least one aromatic group such as phenyl, indenyl, indanyl, tetrahydronaphthalenyl or naphthalenyl group.

For the sake of clarity, by the expression “R¹ and R², are taken together and form C₃₋₁₈ cycloalkyl or C₅₋₁₈ cycloalkenyl group”, it is meant that the carbon atom to which both groups are bonded is included into the C₅₋₁₈ cycloalkyl or C₅₋₁₈ cycloalkenyl group.

The term “alkanediyl” is understood as comprising branched, linear, cyclic and alicyclic alkanediyl group.

The term “oxo group” are understood as comprising any group of formula ═0; i.e. such as a ketone or an aldehyde. In other words, a C₁₋₇ alkyl group optionally substituted by an oxo group is an alkyl group having from 1 to 7 carbons and one of these carbon atoms, even the terminal carbon, may be substituted by a ═O group instead of two hydrogen atoms.

The term “hydrocarbylene group” is understood as divalent groups formed by removing two hydrogen atoms from a hydrocarbon group.

For the sake of clarity, by the term “polyol”, or the similar, it is meant the normal meaning understood by a person skilled in the art, i.e. a compound having at least 2 hydroxy groups. The polyol may comprise further functional groups such as ether, ester or acid groups.

For the sake of clarity, when R^{c} is a group of formula (Ha), then compound of formula (II) is a compound of formula

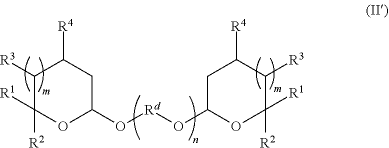

in the form of any one of its stereoisomers or a mixture thereof, and wherein R¹, R², R³, R⁴, R^{d}, n and m have the same meaning as defined above.

According to any embodiment of the invention, the compound (I) can be in the form of a mixture consisting of compound of formula (I′), compound of formula (I″) and compound of formula (I″′) and wherein said compound of formula (I′) represents at least 30% of the total mixture, at least 40%, at least 50%, at least 60%, at least 70%, at least 80%, or even at least 90%. Particularly, the compound of formula (I) is a compound of formula (I′).

According to any embodiment of the invention, when the compound of formula (I) is in the form of a mixture consisting of compound of formula (I′), compound of formula (I″) and compound of formula (I″′), the invention's process may further comprise an isomerisation step to obtain mainly one isomer or increase the amount of one isomer. The person skilled in the art is well aware of the conditions suitable for isomerisation, such as for example under acidic conditions or in presence of metal catalyst either in elemental form or supported or as Rhodium, Ruthenium, Iridium, Platinum or Palladium complex.

According to any embodiment of the invention, when m is 0 the compound of formula (I), the compound of formula (I′), the compound of formula (I″) and the compound of formula (I″′) are respectively compounds of formula

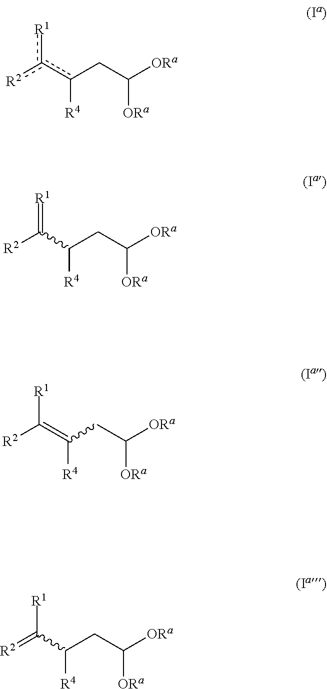

in the form of any one of its stereoisomers or a mixture thereof, and wherein R¹, R², R³, R⁴ and R^{a} have the same meaning as defined above.

According to any embodiment of the invention, R³ may be a hydrogen atom or a C₁₋₄ alkyl group. Particularly, R³ may be a hydrogen atom or a C₁₋₃ alkyl group. Particularly, R³ may be hydrogen atom or a methyl or ethyl group. Even more particularly, R³ may be a hydrogen atom or a methyl group.

According to any embodiment of the invention, m is 0.

According to any embodiment of the invention, R⁴ may be a hydrogen atom or a C₁₋₄ alkyl group. Particularly, R⁴ may be a hydrogen atom or a C₁₋₃ alkyl group. Particularly, R⁴ may be hydrogen atom or a methyl or ethyl group. Even more particularly, R⁴ may be a hydrogen atom.

According to any embodiment of the invention, R^{c} may be a hydrogen atom, a C₂₋₁₀ hydrocarbone group, optionally comprising one to five oxygen atoms or R^{c} may be a group of formula (II^{a}) as defined above. Particularly, R may be a hydrogen atom, a C₂₋₁₀ hydrocarbon group optionally substituted by one oxo group or one or three hydroxy groups and optionally comprising one or two ether functional groups or R^{c} may be a group of formula (II^{a}) as defined above. Particularly, R^{c} may be a hydrogen atom, a C₁₋₈ alkyl group, optionally substituted by an oxo or one or two hydroxy groups or R^{c} may be a group of formula (II^{a}) as defined above. Particularly, R^{c} may be a hydrogen atom, a C₁₋₇ alkyl group optionally substituted by one oxo group or one or two hydroxy groups or R^{c} may be a group of formula (II^{a}) as defined above. Particularly, R^{c} may be a hydrogen atom, a C₁₋₆ alkyl group optionally substituted by one or two hydroxy groups or R^{c} may be a group of formula (II^{a}) as defined above. Particularly, R^{c} may be a hydrogen atom, a C₁₋₄ alkyl group optionally substituted by one or two hydroxy groups or R^{c} may be a group of formula (Ha) as defined above wherein n is 1. Particularly, R^{c} may be a hydrogen atom, a C₁₋₃ alkyl group optionally substituted by one or two hydroxy groups or R^{c} may be a group of formula (Ha) as defined above wherein n is 1. Particularly, R^{c} may a C₁₋₃ alkyl group optionally substituted by one hydroxy group. Even more particularly, R^{c} may be a (CH₂)ₙOH group wherein n is 2 or 3 or 4, particularly n is 2.

According to any embodiment of the invention, R^{d} may be a C₂₋₁₀ hydrocarbylene group, optionally comprising one to five oxygen atoms. Particularly, R^{d} may be a C₂₋₁₀ hydrocarbylene group optionally substituted by one oxo group or one or three hydroxy groups and optionally comprising one or two ether functional groups. Particularly R^{d} may be a C₂₋₈ alkanediyl group, optionally substituted by one oxo group or one or two hydroxy groups. Particularly, R^{d} may be a C₂₋₇ alkanediyl group optionally substituted by one oxo group or one or two hydroxy groups. Particularly, R^{d} may be a C₂₋₆ alkanediyl group optionally substituted by one oxo group or one or two hydroxy groups. Particularly, R^{d} may be a C₂₋₆ alkanedyl group optionally substituted by one oxo or hydroxy group. Particularly, R^{d} may a C₂₋₄ alkanediyl group optionally substituted by oxo or hydroxy group. Particularly, R^{d} may be a C₂₋₃ alkanediyl group optionally substituted by one hydroxy group. Particularly, R^{d} may a C₂₋₃ alkanediyl group optionally substituted by one hydroxy group. Even more particularly, R^{d} may be a (CH₂)ₙ group wherein n is 2 or 3 or 4, particularly n is 2.

According to any embodiment of the invention, the sum of carbon atoms of R¹ and R² groups is at least 5.

According to any embodiment of the invention, R² may be a hydrogen atom or a C₁₋₁₀ hydrocarbon group, optionally comprising one to three oxygen atoms. Particularly, R² may be a hydrogen atom or a C₁₋₁₀ alkyl group, optionally comprising one to two oxygen atoms, Particularly, R² may be a hydrogen atom or a C₁₋₈ alkyl group. Particularly, R² may be a hydrogen atom or a C₁₋₆ alkyl group. Particularly, R² may be a hydrogen atom or a C₁₋₄ alkyl group. Particularly, R² may be a hydrogen atom or a C₁₋₃ alkyl group. Particularly, R² may be hydrogen atom or a methyl or ethyl group. Even more particularly, R² may be a hydrogen atom or a methyl group.

According to any embodiment of the invention, R¹ may be a C₁₋₁₀ hydrocarbon group, optionally comprising one to three oxygen atoms. Particularly, R¹ may be a C₁₋₁₀ alkyl group optionally comprising one to two oxygen atoms or a phenyl or benzyl group optionally substituted by one to three C₁₋₃ alkoxy groups, a C₁₋₆ alkyl groups or a C₂₋₆ alkenyl groups. Particularly, R¹ may be a C₁₋₈ alkyl group optionally comprising one to two oxygen atoms or a phenyl or benzyl group optionally substituted by one or two methoxy or ethoxy groups or C₁₋₄ alkyl groups. Particularly, R¹ may be a C₁₋₆ alkyl group or a phenyl or benzyl group optionally substituted by a C₁₋₃ alkyl group. Even more particularly, R¹ may be a phenyl or benzyl group optionally substituted by a methyl or an ethyl group.

According to any embodiment of the invention, R¹ and R² may be taken together and form a C₅₋₁₆ cycloalkyl or C₅₋₁₆ cycloalkenyl group, each optionally substituted by one or more hydroxy, C₁₋₁₂ alkyl, C₂₋₁₂ alkenyl, C₁₋₁₂ alkoxy, C₃₋₁₂ cycloalkyl, C₅₋₁₂ cycloalkenyl, C₁₋₆ ester, COOH, C₆₋₁₀ aryl and/or C₆₋₁₀ aryloxy groups. Particularly, R¹ and R² are taken together and form a C₅₋₁₄ cycloalkyl or C₅₋₁₄ cycloalkenyl group, each optionally substituted by one or more C₁₋₁₂ alkyl, C₁₋₁₂ alkoxy, C₃₋₁₂ cycloalkyl, C₆₋₁₀ aryl and/or C₆₋₁₀ aryloxy groups. Particularly, R¹ and R² are taken together and form a C₅₋₁₂ cycloalkyl or C₅₋₁₂ cycloalkenyl group, each optionally substituted by one or two C₁₋₁₀ alkyl groups. Particularly, R¹ and R² are taken together and form a C₅₋₁₀ cycloalkyl or C₅. cycloalkenyl group, each optionally substituted by one or two C₁₋₁₀ alkyl groups.

Particularly, R¹ and R² are taken together and form a C₅₋₈ cycloalkyl or C₅₋₈ cycloalkenyl group, each optionally substituted by one or two C₁₋₁₀ alkyl groups. Particularly, R¹ and R² are taken together and form a C₅₋₆ cycloalkyl or C₅₋₆ cycloalkenyl group, each optionally substituted by one or two C₁₋₈ alkyl groups. Particularly, R¹ and R² are taken together and form a C₅₋₆ cycloalkyl or C₅₋₆ cycloalkenyl group, each optionally substituted by one or two C₁₋₆ alkyl group. Particularly, R¹ and R² are taken together and form a C₅₋₆ cycloalkyl or C₅₋₆ cycloalkenyl group, each optionally substituted by one or two C₁₋₄ alkyl groups. Particularly, R¹ and R² are taken together and form a C₅₋₆ or C₅₋₆ cycloalkenyl cycloalkyl group, each optionally substituted by one or two methyl, ethyl, propyl, isopropyl, butyl, isobutyl, sec butyl or tert butyl groups.

According to any embodiment of the invention, the compound of formula (I) is of formula

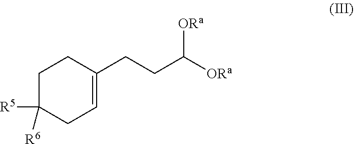

in the form of any one of its stereoisomers or a mixture thereof, and wherein each R^{a} has the same meaning as defined above and R⁵ and R⁶, independently from each other, represent a hydrogen atom, a C₁₋₆ alkyl group;
and said compound of formula (II) is of formula

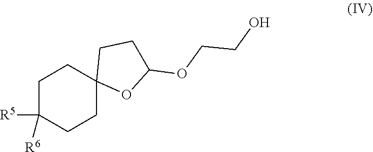

in the form of any one of its stereoisomers or a mixture thereof, and wherein each R⁵ and R⁶ have the same meaning as defined above.

According to any embodiment of the invention, R⁶ may be a hydrogen atom or a C₁₋₄ alkyl group. Particularly, R⁶ may be a hydrogen atom or a C₁₋₃ alkyl group.

Particularly, R⁶ may be hydrogen atom or a methyl or ethyl group. Even more particularly, R⁶ may be a methyl group.

According to any embodiment of the invention, R⁵ may be a hydrogen atom or a C₁₋₄ alkyl group. Particularly, R⁵ may be a hydrogen atom or a C₁₋₃ alkyl group.

Particularly, R⁵ may be hydrogen atom or a methyl or ethyl group. Even more particularly, R⁵ may be a methyl group.

According to any embodiment of the invention, the acid is a Lewis acid or a Bronsted acid. Said acid has a pH below 3. Non-limiting examples of acid suitable for the invention's process may be selected from the group consisting of pTsOH, MsOH, TfOH, H₂SO₄, HCl, H₃PO₄, KHSO₄, NaHSO₄, BF₃Et₂O, BF₃ (AcOH)₂, ZnBr₂, Fe(OTf)₃, Fe(OTs)₃, FeCl₃, Zn(OTf)₂, Bi(OTf)₃, Cu(OTf)₂, Al(OTf)₃, SnCl₄, Alox acidic (Axsorb A2-5, Al₂O₃ 504C), Amberlyst 15, Amberlyst 35, SiO₂, Wayphos, polyphosphoric acid, Zeolite (CBV 21A sold by Zeolyst, CBV 780 sold by Zeolyst, CP814E sold by Zeolyst), boric acid, CSA, K10-S300 (Bentonite) sold by Clariant, F24 X (Clay) sold by EP minerals, F21 X (Clay) sold by EP minerals, Siral® 40 HPV sold by Sasol.

The acid can be added into the reaction medium of the invention's process in a large range of concentrations. As non-limiting examples, one can cite as acid concentration values those ranging from about 1 to about 100 mol %, preferably from 1 to about 50 mol %, relative to the amount of the compound of formula (II), preferably from 1.5 to about 2.5 mol %, relative to the amount of the compound of formula (II). The optimum concentration of the acid will depend, as the person skilled in the art knows, on the nature of the latter, on the nature of the compound of formula (II), on the reaction temperature as well as on the desired time of reaction.

According to any embodiment of the invention, the polyol used in the process to prepare compound of formula (I) is a diol, triol, tetraol or pentaol. In particular, the polyol is a compound of formula HOROH wherein R is a C₂₋₁₂ hydrocarbylene group, optionally comprising one to five oxygen atoms, particularly, wherein R is a C₂₋₁₀ hydrocarbylene group optionally comprising one to five oxygen atoms, particularly wherein R is a C₂₋₁₀ hydrocarbylene group optionally substituted by one oxo group or one to three hydroxy groups and optionally comprising one or two ether functional groups, particularly R is a C₂₋₈ alkanediyl group optionally substituted by one or two hydroxy groups and optionally comprising an ether functional group, particularly R may be a C₂₋₆ alkanediyl group optionally substituted by one or two hydroxy groups, particularly R may be a C₂₋₄ alkanediyl group optionally substituted by a hydroxy group, particularly R may be a linear C₂₋₄ alkanediyl group optionally substituted by a hydroxy group, particularly R may be a linear C₂₋₃ alkanediyl group optionally substituted by a hydroxy group, even more particularly, R may be a 1,2-ethanediyl group. Particularly, the polyol is a diol, in particular a 1,2-alkanediol, a 1,3-alkanediol or a 1,4-alkanediol. Non-limiting examples of diol suitable for the invention's process may be selected from the group consisting of ethylene glycol, propane-1,2-diol, propane-1,3-diol, butane-2,3-diol, 1,2-butanediol, 1,2-pentandiol, 2,3-dimethyl-3-hydroxy-2-butanol, glycerol, diglycerol, triglycerol, erythritol, threitol, trans-1,2-cyclohexandiol, cis-1,2-cyclohexanol, 1,2-cyclopentanol, neopentylglycol, 2-methyl-2-propyl-1,3-propanediol, 2-methyl-1,2-propanediol, 2,2-dimethyl-1,3-propanediol, pentane-2,4-diol, 2-methylpentane-2,4-diol, butane-1,3-diol, 3-methylbutane-1,3-diol, 2-methylpropane-1,3-diol, 2-methylpropane-1,2,3-triol, 1,3-dihydroxypropan-2-one, 1,4-butandiol, hexane-2,5-diol, 2,5-dimethylhexane-2,5-diol, 2-methylhexane-2,5-diol, 2,2-dimethylbutane-1,4-diol, 1,2-benzenedimethanol, 2-hydroxybenzyl alcohol, (Z)-but-2-ene-1,4-diol, pentaerythritol. Particularly, the diol may be selected from the group consisting of ethylene glycol, 2,2-dimethylpropane-1,3-diol and propane-1,3-diol.

The polyol, such as the diol, triol or tetraol, can be added into the reaction medium of the invention's process in a large range of concentrations. As non-limiting examples, one can cite as diol or triol concentration values those ranging from about 1 to about 12 equivalents, relative to the amount of the compound of formula (II), preferably from 2 to about 12 equivalents, relative to the amount of the compound of formula (II), preferably from 3 to about 5 equivalents, relative to the amount of the compound of formula (II). The optimum concentration of the diol or triol will depend, as the person skilled in the art knows, on the nature of the latter, on the nature of the compound of formula (II), on the reaction temperature as well as on the desired time of reaction.

According to any embodiment of the invention, the alpha hydroxy carboxylic acid is of formula R′CH(OH)COOH wherein R′ is a hydrogen atom or a C₁₋₆ alkyl group, particularly R′ may be a hydrogen atom or a C₁₋₄ alkyl group, particularly R^{c} may be a hydrogen atom or a C₁₋₃ alkyl group, particularly R′ may be a hydrogen atom or a C₁₋₂ alkyl group, even more particularly, R′ may be a hydrogen atom or a methyl group. Non-limiting examples of alpha hydroxy carboxylic acids suitable for the invention's process may be selected from the group consisting of lactic acid, and glycolic acid.

The alpha hydroxy carboxylic acid can be added into the reaction medium of the invention's process in a large range of concentrations. As non-limiting examples, one can cite as alpha hydroxy carboxylic acid concentration values those ranging from about 2 to about 12 equivalents, relative to the amount of the compound of formula (II), preferably from 3 to about 5 equivalents, relative to the amount of the compound of formula (II). The optimum concentration of the alpha hydroxy carboxylic acid will depend, as the person skilled in the art knows, on the nature of the latter, on the nature of the compound of formula (II), on the reaction temperature as well as on the desired time of reaction.

According to any embodiment of the invention, both R^{a} are taken together and represent a C₂₋₁₈ hydrocarbylene group, optionally comprising one to five oxygen atoms. Particularly, both R^{a} are taken together and represent a C₂₋₁₆ hydrocarbylene group, optionally comprising one to five oxygen atoms. Particularly, both R^{a} are taken together and represent a C₂₋₁₄ hydrocarbylene group, optionally comprising one to five oxygen atoms. Particularly, both R^{a} are taken together and represent a C₂₋₁₂ hydrocarbylene group, optionally comprising one to five oxygen atoms. Particularly, both R^{a} are taken together and represent a C₂₋₁₀ hydrocarbylene group, optionally comprising one to five oxygen atoms. Particularly, both R^{a} are taken together and represent a C₂₋₁₀ hydrocarbylene group optionally substituted by one oxo group or one or three hydroxy groups and optionally comprising one or two ether functional groups, particularly both R^{a} are taken together and represent a C₂₋₈ alkanediyl group, optionally substituted by an oxo or one or two hydroxy groups. Particularly, both R^{a} are taken together and represent a C₂₋₈ alkanediyl group, optionally substituted by an oxo or hydroxy group. Particularly, both R^{a} may be taken together and represent a C₂₋₆ alkanediyl group, optionally substituted by an oxo or hydroxy group. Particularly, both R^{a} may be taken together and represent a C₂₋₄ alkanediyl group, optionally substituted by an oxo or hydroxy group. Particularly, both R^{a} may be taken together and represent a C₂₋₃ alkanediyl group, optionally substituted by an oxo or hydroxy group. Even more particularly, R^{a} may be taken together and represent a 1,2-ethanediyl, 1,3-propanediyl or a 2,2-dimethylpropane-1,3-diyl group.

According to a particular embodiment of the invention, when the polyol is a tetraol, the compound of formula (I) may be in a form of a dimer of formula

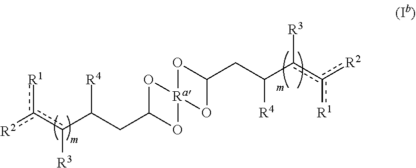

wherein the dotted lines, R¹, R², R³, R⁴ and m have the same meaning as defined in formula (I) and R^{a} is a C₂₋₈ alkanetetrayl group, particularly, R^{a}′ is C₃₋₆ alkanetetrayl group.

According to a particular embodiment of the invention, the compound of formula

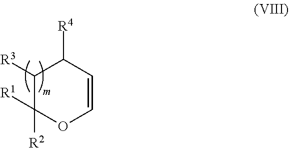

in the form of any one of its stereoisomers or a mixture thereof, and wherein R¹,
R², R³, R⁴ and m have the same meaning as defined in formula (I); may be formed during the invention process. Compound of formula (VIII), under invention's process is converted into compound of formula (II).

According to any one of the invention's embodiments, the invention's process to prepare compound of formula (I) is carried out at a temperature comprised between 20° C. and 250° C. In particular, the temperature is in the range between 90° C. and 145° C. Of course, a person skilled in the art is also able to select the preferred temperature as a function of the melting and boiling point of the starting and final products as well as the desired time of reaction or conversion.

The invention's process to prepare compound of formula (I) can be carried out in the presence or absence of a solvent. When a solvent is required or used for practical reasons, then any solvent current in such reaction type can be used for the purposes of the invention. Non-limiting examples include C₆₋₁₂ aromatic solvents such as toluene, xylene, 1,3-diisopropylbenzene, cumene, chlorobenzene or pseudocumene, or mixtures thereof, hydrocarbon solvents such as cyclohexane or heptane, octane or ethereal solvents as methyl tetrahydrofuran. The choice of the solvent is function of the nature of the substrate and/or catalyst and the person skilled in the art is well able to select the solvent most suitable in each case to optimize the reaction.

According to any one of the invention's embodiments, the water generated during the invention's process is removed continuously during the invention's process. The removal of water formed during the invention process may be done by distillation, azeotropic distillation, addition of a drying agent or orthoformiate.

Non-limiting examples of suitable compounds of formula (I) may include 2-(2-(4,4-dimethylcyclohex-1-en-1-yl)ethyl)-1,3-dioxolane, 2-(2-(4,4-dimethylcyclohex-1-en-1-yl)ethyl)-1,3-dioxane, 2-(2-(4,4-dimethylcyclohex-1-en-1-yl)ethyl)-4-propyl-1,3-dioxolane, 2-(2-(4,4-dimethylcyclohex-1-en-1-yl)ethyl)-4,5-dimethyl-1,3-dioxolane, 2-(2-(4,4-dimethylcyclohex-1-en-1-yl)ethyl)-5,5-dimethyl-1,3-dioxane, 3,9-bis(2-(4,4-dimethylcyclohex-1-en-1-yl)ethyl)-2,4,8,10-tetraoxaspiro[5.5]undecane, 2-(3-(4,4-dimethylcyclohex-1-en-1-yl)propyl)-1,3-dioxolane, (E)-2-(3,7-dimethyloct-3-en-1-yl)-1,3-dioxolane/(Z)-2-(3,7-dimethyloct-3-en-1-yl)-1,3-dioxolane/(E)-2-(3,7-dimethyloct-2-en-1-yl)-1,3-dioxolane/(Z)-2-(3,7-dimethyloct-2-en-1-yl)-1,3-dioxolane/2-(7-methyl-3-methyleneoctyl)-1,3-dioxolane, 2-(2-(4,4-dimethylcyclohex-1-en-1-yl)ethyl)-1,3-dioxan-5-ol, (2-(2-(4,4-dimethylcyclohex-1-en-1-yl)ethyl)-1,3-dioxolan-4-yl)metanol,_2-(2-(4-(tert-butyl)cyclohex-1-en-1-yl)ethyl)-1,3-dioxolane, 2-(2-(5-isopropylcyclohex-1-en-1-yl)ethyl)-1,3-dioxolane,2-(2-(3-isopropylcyclohex-1-en-1-yl)ethyl)-1,3-dioxolane, 2-(2-(4-isopropylcyclohex-1-en-1-yl)ethyl)-1,3-dioxolane, 2-(2-(4-butylcyclohex-1-en-1-yl)ethyl)-1,3-dioxolane, 2-(2-(4-isopropylcyclopent-1-en-1-yl)ethyl)-1,3-dioxolane, 2-(2-(3-isopropylcyclopent-1-en-1-yl)ethyl)-1,3-dioxolane, (E)-2-(3-methyl-4-(p-tolyl)but-3-en-1-yl)-1,3-dioxolane, (Z)-2-(3-methyl-4-(p-tolyl)but-3-en-1-yl)-1,3-dioxolane, (E/Z)-2-(3-methyl-4-(p-tolyl)but-2-en-1-yl)-1,3-dioxolane, 2-(3-(4-methylbenzyl)but-3-en-1-yl)-1,3-dioxolane. Non-limiting examples of suitable compounds of formula (II) may include 2-((8,8-dimethyl-1-oxaspiro[4.5]decan-2-yl)oxy)ethan-1-ol, 3-((8,8-dimethyl-1-oxaspiro[4.5]decan-2-yl)oxy)propan-1-ol, 1-((8,8-dimethyl-1-oxaspiro[4.5]decan-2-yl)oxy)pentan-2-ol, 3-((8,8-dimethyl-1-oxaspiro[4.5]decan-2-yl)oxy)butan-2-ol, 3-((8,8-dimethyl-1-oxaspiro[4.5]decan-2-yl)oxy)-2,2-dimethylpropan-1-ol, 2-(((8,8-dimethyl-1-oxaspiro[4.5]decan-2-yl)oxy)methyl)-2-(hydroxymethyl)propane-1,3-diol, 2-((9,9-dimethyl-1-oxaspiro[5.5]undecan-2-yl)oxy)ethan-1-ol, 2-((5-methyl-5-(4-methylpentyl)tetrahydrofuran-2-yl)oxy)ethan-1-ol, 3-((8,8-dimethyl-1-oxaspiro[4.5]decan-2-yl)oxy)propane-1,2-diol, 2-((8-(tert-butyl)-1-oxaspiro[4.5]decan-2-yl)oxy)ethan-1-ol, 2-((7-isopropyl-1-oxaspiro[4.5]decan-2-yl)oxy)ethan-1-ol, 2-((8-isopropyl-1-oxaspiro[4.5]decan-2-yl)oxy)ethan-1-ol, 2-((8-butyl-1-oxaspiro[4.5]decan-2-yl)oxy)ethan-1-ol, 2-((7-isopropyl-1-oxaspiro[4.4]nonan-2-yl)oxy)ethan-1-ol, 2-((5-methyl-5-(4-methylbenzyl)tetrahydrofuran-2-yl)oxy)ethan-1-ol, 2-((5-methyl-6-(p-tolyl)tetrahydro-2H-pyran-2-yl)oxy)ethan-1-ol.

According to any embodiment of the invention, the invention's process for the preparation of a compound of formula (I) may be carried out under batch and/or continuous conditions.

According to any embodiment of the invention, the compound of formula (I) may be converted into a compound of formula

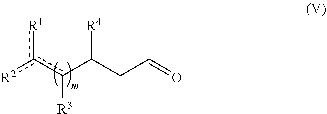

in the form of any one of its stereoisomers or a mixture thereof, and wherein the dotted lines, m, R¹, R², R³ and R⁴ have the same meaning as defined above. The conversion of compound of formula (I) into compound of formula (V) is a deprotection that has been largely reported in the prior arts. According to any embodiments of the invention, the deprotection of the acetal group to obtain compound of formula (V) may be carried out under normal condition known by the person skilled in the art, i.e. with a large molar excess of carboxylic acid in water. Specific and non-limiting examples of carboxylic acids may be selected from the group consisting of acetic acid, propionic acid, citric acid, formic acid, TFA, oxalic acid or a mixture thereof. Optionally, the deprotection may be carried out in the presence of ethylene glycol or a C₁₋₆ alcohol, such as methanol, ethanol or propanol and in the presence of a strong acid.

The carboxylic acid, used in the deprotection, can be added into the reaction medium of the invention's process in a large range of concentrations. As non-limiting examples, one can cite as acid concentration values those ranging from about 5 to about equivalents, relative to the amount of the of substrate, preferably from 5 to about 10 equivalents, relative to the amount of the of substrate. The optimum concentration of the acid will depend, as the person skilled in the art knows, on the nature of the latter, on the nature of the substrate, on the reaction temperature as well as on the desired time of reaction.

The ethylene glycol or a C₁₋₆ alcohol, used in the deprotection, can be added into the reaction medium of the invention's process in a large range of concentrations. As non-limiting examples, one can cite as ethylene glycol or a C₁₋₆ alcohol concentration values those ranging from about 5 to about 20 equivalents, relative to the amount of the of substrate, preferably from 5 to about 10 equivalents, relative to the amount of the of substrate. The optimum concentration of the acid will depend, as the person skilled in the art knows, on the nature of the latter, on the nature of the substrate, on the reaction temperature as well as on the desired time of reaction. The strong acid can be added into the reaction medium of the invention's process in a large range of concentrations. As non-limiting examples, one can cite as acid concentration values those ranging from about 1 mol % to about 50 mol % equivalents, relative to the amount of the of substrate, preferably from 5 to about 10 mol %, relative to the amount of the of substrate.

According to any one of the invention's embodiments, the deprotection to form compound of formula (V) may be carried out at a temperature comprised between 40° C. and 120° C. In particular, the temperature is in the range between 70° C. and 90° C. Of course, a person skilled in the art is also able to select the preferred temperature as a function of the melting and boiling point of the starting and final products as well as the desired time of reaction or conversion.

According to any one of the invention's embodiments, the deprotection to form compound of formula (V) can be carried out in the presence or absence of a solvent. When a solvent is required or used for practical reasons, then any solvent current in such reaction type can be used for the purposes of the invention. Non-limiting examples include C₆₋₁₂ aromatic solvents such as toluene, xylene, 1,3-diisopropylbenzene, cumene or pseudocumene, or mixtures thereof, alcoholic solvents such as methanol, ethanol, 2-methylbutan-2-ol or mixtures thereof, hydrocarbon solvents such as cyclohexane, heptane or mixtures thereof, esteric solvents such as n-butyl acetate, iso-propyl acetate, ethyl acetate or ethereal solvents such as methyl tetrahydrofuran, tetrahydrofuran or mixtures thereof. The choice of the solvent is a function of the nature of the substrate and of the carboxylic derivative and the person skilled in the art is well able to select the solvent most convenient in each case to optimize the reaction.

According to any embodiment of the invention, the compound of formula (H) may be prepared by hydroformylation. So another object of the present invention is a process for the preparation of a compound of formula

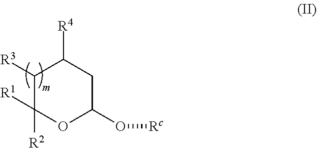

in the form of any one of its stereoisomers or a mixture thereof, and wherein m is 0 or 1; R¹ represents a C₁₋₁₂ hydrocarbon group, optionally comprising one to three oxygen atoms and/or one to two nitrogen atoms and/or one sulfur atom; R² represents hydrogen atom or a R¹ group; or R¹ and R² are taken together and form C₅₋₁₈ cycloalkyl or C₅₋₁₈ cycloalkenyl group, each optionally substituted by one or more hydroxy, C₁₋₁₅ alkyl, C₂₋₁₅ alkenyl, C₁₋₁₅ alkoxy, C₃₋₁₅ cycloalkyl, C₅₋₁₅ cycloalkenyl, C₁₋₆ ester, COOH, C₆₋₁₀ aryl and/or C₆₋₁₀ aryloxy group; R³ and R⁴, independently from each other, represent a hydrogen atom or a C₁₋₆ alkyl group; and R^{c} is a is a hydrogen atom, a C₁₋₁₂ hydrocarbon group optionally comprising one to five oxygen atoms or R^{c} is a group of formula

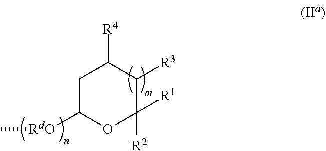

wherein R¹, R², R³, R⁴ and m have the same meaning as defined in formula (H); n is 0 or 1, R^{d} is a C₁₋₁₂ hydrocarbylene group optionally comprising one to five oxygen atoms and the hatched line indicates the location of the between (II^{a}) and the oxygen atom of formula (II);
by the hydroformylation of a compound of formula

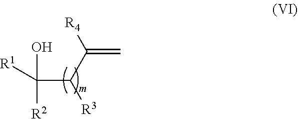

in the form of any one of its stereoisomers or a mixture thereof, and wherein R¹,
R², R³, R⁴ and m have the same meaning as defined in formula (II);
in a presence of a hydroformylation catalyst comprising a bidentate phosphorous ligand and optionally an alcohol or a polyol.

For the sake of clarity, by the expression “hydroformylation”, or the similar, it is meant the normal meaning understood by a person skilled in the art, i.e. the reaction is performed in a presence of a metal catalyst such as a Rhodium, Cobalt or Platinum complex, preferably a Rhodium complex, carbon monoxide, hydrogen and a bidentate phosphorous ligand.

According to any embodiments of the invention, the polyol used in the preparation of compound of formula (II); i.e. in the hydroformylation, may be a C₁₋₁₀ hydrocarbon comprising at least 2 hydroxy groups. Particularly, the polyol may be a C₁₋₁₀ hydrocarbon comprising 2 to 4 hydroxy groups. Particularly, the polyol may be a diol or a triol having 1 to 8 carbon atoms. Particularly the polyol may be a diol.

According to any embodiment of the invention, the hydroformylation of compound of formula (VI) may be carried out in the presence of an alcohol or a polyol.

According to any embodiment of the invention, the diol is of formula HOR^{d}OH wherein R^{d} may be a C₂₋₁₀ hydrocarbylene group, optionally comprising one to five oxygen atoms; particularly, R^{d} may be a hydrogen atom, a C₂₋₁₀ hydrocarbylene group optionally substituted by one oxo group or one or three hydroxy groups and optionally comprising one or two ether functional groups; particularly R^{d} may be a C₂₋₈ alkanediyl group, optionally substituted by an oxo or one or two hydroxy groups; particularly, R^{d} may be a C₂₋₇ alkanediyl group optionally substituted by one oxo group or one or two hydroxy groups; particularly, R^{d} may be a C₂₋₆ alkanediyl group optionally substituted by one oxo group or one or two hydroxy groups; R^{d} is a C₂₋₆ alkanediyl group optionally substituted by one oxo or hydroxy group, particularly R^{d} may be a C₂₋₄ alkanediyl group, particularly R^{d} may be a linear C₂₋₄ alkanediyl group, even more particularly, R^{d} may be a 1,2-ethanediyl group. Non-limiting examples of diol suitable for the invention's process may be selected from the group consisting of ethylene glycol, propane-1,2-diol, propane-1,3-diol, butane-2,3-diol.

According to any embodiment of the invention, the alcohol is of formula R^{c}OH wherein R^{e} is a C₁₋₆ alkyl group, particularly R^{e} may be a C₁₋₄ alkyl group, particularly R^{e} may be a C₁₋₃ alkyl group, particularly R^{e} may be a C₁₋₁₂ alkyl group, even more particularly, R^{e} may be a methyl group. Non-limiting examples of alcohol suitable for the invention's process may be selected from the group consisting of methanol, ethanol, propanol and butanol.

The diol or the alcohol can be added into the reaction medium of the invention's process in a large range of concentrations. As non-limiting examples, one can cite as diol or alcohol concentration values those ranging from about 1 to about 12 equivalents, relative to the amount of the compound of formula (VI), preferably from about 3.4 to about 12 equivalents, relative to the amount of the compound of formula (VI), preferably from 3.5 to about 5 equivalents, relative to the amount of the compound of formula (VI). Particularly, the alcohol or diol is added into the reaction medium at a concentrations of at least 3.4 equivalents, relative to the amount of the compound of formula (VI). The optimum concentration of the diol will depend, as the person skilled in the art knows, on the nature of the latter, on the nature of the compound of formula (VI), on the reaction temperature, the crystallization behavior of the reaction products as well as on the desired time of reaction.

According to any embodiment of the invention, the hydroformylation is performed in a presence of a Rhodium complex. The Rhodium complexes that can be used in the present invention include but are not limited to Rh(acac)(CO)₂, RhCl₃, Rh₂AcO₄, [Rh(OAc)(COD)]₂, Rh₄(CO)₁₂, Rh₆(CO)₁₆, RhCl(CO)(PPh₃)₂, Rh(C₂H₄)₂(acac), [Rh(Cl)(COD)]₂, [Rh(Cl)(COE)₂]₂, [Rh(OAc)(CO)₂]₂, Rh(acac)(COD), HRh(CO)(PPh₃)₃, RhCl(PPh₃)₃, [Rh(NBD)₂]BF₄, [Rh(OMe)(COD)]₂ and [Rh(OH)(COD)]₂ wherein acac represents an acetyl acetonate group, Ac an acetyl group, COD a 1,5-cyclooctadiene group, COE a cyclooctene group, Ph a phenyl group. Particularly, the Rhodium complex may be selected from the group consisting of Rh(acac)(CO)₂, [Rh(OAc)(COD)]₂, RhCl(CO)(PPh₃)₂, Rh(C₂H₄)₂(acac), [Rh(Cl)(COD)]₂, [Rh(Cl)(COE)₂]₂, [Rh(OAc)(CO)₂]₂, Rh(acac)(COD), HRh(CO)(PPh₃)₃, RhCl(PPh₃)₃, [Rh(NBD)₂]BF₄, [Rh(OMe)(COD)]₂, and [Rh(OH)(COD)]₂. Even more particularly, the Rhodium complex may be selected from the group consisting of Rh(acac)(CO)₂, Rh(acac)(COD), HRh(CO)(PPh₃)₃, [Rh(OMe)(COD)]₂ and [Rh(OH)(COD)]₂. Said complex can be added into the reaction medium of the invention's process in a large range of concentrations. As non-limiting examples, one can cite as complex concentration values those ranging from about 0.0005 mol % to about 5 mol %, relative to the amount of substrate, preferably from 0.001 mol % to about 5 mol %, relative to the amount of substrate. Preferably, the complex concentration will be comprised between 0.0025 mol % to 2 mol %. It goes without saying that the optimum concentration of the complex will depend, as the person skilled in the art knows, on the nature of the latter, on the nature of the substrate, on the nature of the ligand, on the reaction temperature as well as on the desired time of reaction.

According to any embodiment of the invention, the hydroformylation is performed in a presence of bidentate phosphorous ligand.

According to any one of the above embodiments, the hydroformylation may be performed in presence of a bidentate phosphorous ligand 3,9-bis[2,4-bis(1-methyl-1-phenyl-ethyl)phenoxy]-2,4,8,10-tetraoxa-3,9-diphosphaspiro[5.5]undecane or of formula

(R⁹)₂P-Q-P(R⁹)₂ (A)
wherein each R⁹, taken separately, represents a C₄ heteroaryl group, a C₆₋₁₀ aromatic group optionally substituted or a cyclohexyl group optionally substituted, or the two R⁹ bonded to the same P atom, taken together, represent a 1,1′-biphenyl-2,2′-dioxy, a 1,1′-biphenyl-2,2′-dimetyl or a 1,1′-biphenyl]-2,2′-diyl, each optionally substituted; and
Q represents a group of formula

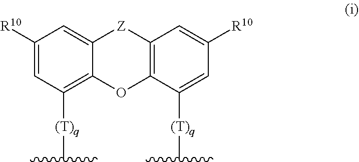

wherein q is 0 or 1, each T, independently from each other, represent an oxygen atom or a CH₂ group, each R¹⁰, independently from each other, represents a hydrogen atom or a C₁₋₈ alkyl group, and Z represents an oxygen or sulfur atom or a C(R¹¹)₂, Si(R¹²)₂ or NR¹¹ group, in which R¹¹ is a hydrogen atom or a R² group, R¹² representing a C₁₋₄ linear or branched alkyl group, preferably methyl group; or

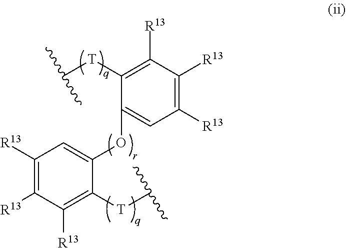

in the form of any one of its enantiomers, and wherein q is 0 or 1, r is 0 or 1, each T, independently from each other, represent an oxygen atom or a CH₂ group, R¹³ independently from each other, represent a hydrogen atom, a methoxy group or a C₁₋₄ alkyl optionally substituted by one to three halogen atom or alkoxy group; or two adjacent R¹³ may be taken together and represent a (CH)₄ group;

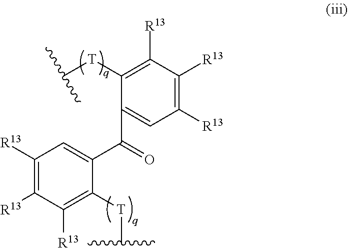

in the form of any one of its enantiomers, and wherein T, q and R¹³ have the same meaning as above;
and the wavy lines indicate the position of the bond between said Q group and the rest of the compound (A).

According to any one of the above embodiments, Q may be a group of formula (i) or (ii).

According to any one of the above embodiments, each R⁹ may be a furan-2-yl group, a 1H-pyrrol-1-yl group a C₆₋₁₀ aromatic group optionally substituted or a cyclohexyl group optionally substituted.

According to any one of the above embodiments, by “aromatic group or ring” it is meant a phenyl or naphthyl group, and in particular a phenyl group.

According to any one of the above embodiments, each R⁹ may be a phenyl group, a cyclohexyl group, a 3,5-dimethyl-phenyl, a 3,5-di(CF₃)-phenyl, a 3,5-dimethyl-4-methoxy-phenyl group.

According to any one of the above embodiments, the R¹⁰ may be a hydrogen atom.

According to any one of the above embodiments, Z may be a CMe₂, SiMe₂, NH or NMe group. Particularly, Z may be a CMe₂ group.

According to any one of the above embodiments, non-limiting examples of possible substituents of R⁹ are one, two, three or four groups selected amongst the halogen atoms, or C₁₋₁₀ alkoxy, alkyl, alkenyl, pyridyl or perhalo-hydrocarbon group. Two substituents may be taken together to form a C₄₋₈ cycloalkyl group. The expression “perhalo-hydrocarbon” has here the usual meaning in the art, e.g. a group such as CF₃ for instance. In particular said substituents are one or two halogen atoms, such as F or C₁, or C₁₋₄ alkoxy or alkyl groups, or CF₃ groups.

According to any one of the above embodiments, said R⁹, may be non-substituted.

According to any one of the above embodiments, the ligand of formula (A) can be in a racemic or optically active form.

Non limiting example of bidendate phosphorous ligand may include 2,2′-bis((di(1H-pyrrol-1-yl)phosphanyl)oxy)-1,1′-binaphthalene, 1,1′-((naphthalen-2-yloxy)phosphanediyl)bis(1H-pyrrole), 2,2′-bis((di(1H-pyrrol-1-yl)phosphanyl)oxy)-1,1′-biphenyl, (9,9-dimethyl-9H-xanthene-4,5-diyl)bis(diphenylphosphine), 2,2′-bis((di(1H-pyrrol-1-yl)phosphaneyl)oxy)-5,5′,6,6′,7,7′,8,8′-octahydro-1,1′-binaphthalene. 1,1′,1″,1′-(((2,7-di-tert-butyl-9,9-dimethyl-9H-xanthene-4,5- diyl)bis(oxy))bis(phosphanetriyl))tetrakis(1H-pyrrole), 6,6′-[(3,3′-Di-tert-butyl-5,5′-dimethoxy-1,1′-biphenyl-2,2′-diyl)bis(oxy)]bis(dibenzo[df][1,3,2]dioxaphosphepin), (Oxydi-2,1-phenylene)bis(diphenylphosphine), 2,2′-Bis(diphenylphosphinomethyl)-1,1′-biphenyl, 4,6-bis(diphenylphosphanyl)-10H-phenoxazine, 2-((3,3′-di-tert-butyl-2′-((4,8-di-tert-butyl-2,10-dimethoxydibenzo[df][1,3,2]dioxaphosphepin-6-yl)oxy)-5,5′-dimethoxy-[1,1′-biphenyl]-2-yl)oxy)-4H-naphtho[2,3-d][1,3,2]dioxaphosphinin-4-one, 2-((3,3′-di-tert-butyl-2′-((4,8-di-tert-butyl-2,10-dimethoxydibenzo[df][1,3,2]dioxaphosphepin-6-yl)oxy)-5,5′-dimethoxy-[1,1′-biphenyl]-2-yl)oxy)-8-methyl-4H-benzo[d][1,3,2]dioxaphosphinin-4-one, (1S,1'S)-(−)-(2,7-di-tert-butyl-9,9-dimethyl-9H-xanthene-4,5-diyl)bis((1-naphthyl) (phenyl)phosphine), (1S,1'S)-(−)-(2,7-di-tert-butyl-9,9-dimethyl-9H-xanthene-4,5-diyl)bis((4-methylphenyl) (phenyl)phosphine), 8-methyl-2-((3,3′,5,5′-tetra-tert-butyl-2′-((2,4,8,10-tetra-tert-butyldibenzo[d,f][1,3,2]dioxaphosphepin-6-yl)oxy)-[1,1′-biphenyl]-2-yl)oxy)-4H-benzo[d][1,3,2]dioxaphosphinin-4-one, 2-((3,3′-di-tert-butyl-2′-((4,8-di-tert-butyl-2,10-dimethoxydibenzo[df][1,3,2]dioxaphosphepin-6-yl)oxy)-5,5′-dimethoxy-[1,1′-biphenyl]-2-yl)oxy)-8-isopropyl-5-methyl-4H-benzo[d][1,3,2]dioxaphosphinin-4-one, (1S,1'S)-(+)-(9,9-Dimethyl-9H-xanthene-4,5-diyl)bis((2-methoxyphenyl)(phenyl) phosphine), (1S,1'S)-(+)-(2,7-di-tert-butyl-9,9-dimethyl-9H-xanthene-4,5-diyl)bis((2-methoxyphenyl)(phenyl)phosphine), (1S,1'S)-(+)-(9,9-Dimethyl-9H-xanthene-4,5-diyl)bis((2-methylphenyl)(phenyl) phosphine), (1S,1'S)-(−)-(9,9-Dimethyl-9H-xanthene-4,5-diyl)bis(naphthalen-2-yl(phenyl)phosphine), (1S,1'S)-(−)-(9,9-Dimethyl-9H-xanthene-4,5-diyl)bis((4-methoxyphenyl)(phenyl) phosphine), (1S,1'S)-(−)-(2,7-di-tert-butyl-9,9-dimethyl-9H-xanthene-4,5-diyl)bis((2-naphthyl) (phenyl)phosphine), (1S,1'S)-(−)-(9,9-Dimethyl-9H-xanthene-4,5-diyl)bis(naphthalen-1-yl(phenyl)phosphine), (1S,1'S)-(+)-(2,7-di-tert-butyl-9,9-dimethyl-9H-xanthene-4,5-diyl)bis((2-isopropoxyphenyl)(phenyl)phosphine), (1S,1'S)-(+)-(2,7-di-tert-butyl-9,9-dimethyl-9H-xanthene-4,5-diyl)bis((2-isopropylphenyl)(phenyl)phosphine) or (1S,1'S)-(−)-(2,7-di-tert-butyl-9,9-dimethyl-9H-xanthen-4,5-diyl)bis((dibenzo[b,d]-furan-4-yl)(phenyl)phosphine), (2,7-di-tert-butyl-9,9-dimethyl-9H-xanthene-4,5-diyl)bis((4-methoxyphenyl)(phenyl)phosphane), (4,4′,6,6′-Tetramethoxybiphenyl-2,2′-diyl) bis{bis[3,5-bis(trifluoromethyl)phenyl]phosphine}, (6,6′-dimethoxy-[1,1′-biphenyl]-2,2′-diyl)bis(di(furan-2-yl)phosphane) and 3,9-bis(2,4-bis(2-phenylpropan-2-yl)phenoxy)-2,4,8,10-tetraoxa-3,9-diphosphaspiro[5.5]undecane.

Particularly, the ligand is a bidentate phosphorous ligand which may be selected from the group consisting of (9,9-dimethyl-9H-xanthene-4,5-diyl)bis(diphenylphosphine), 1,1′,1″,1″′-(((2,7-di-tert-butyl-9,9-dimethyl-9H-xanthene-4,5-diyl)bis(oxy))bis(phosphanetriyl))tetrakis(1H-pyrrole), 6,6′-[(3,3′-Di-tert-butyl-5,5′-dimethoxy-1,1′-biphenyl-2,2′-diyl)bis(oxy)]bis(dibenzo[df][1,3,2]dioxaphosphepin), (Oxydi-2,1-phenylene)bis(diphenylphosphine), 2,2′-Bis(diphenylphosphinomethyl)-1,1′-biphenyl, 4,6-bis(diphenylphosphanyl)-10H-phenoxazine, 2-((3,3′-di-tert-butyl-2′-((4,8-di-tert-butyl-2,10-dimethoxydibenzo[df][1,3,2]dioxaphosphepin-6-yl)oxy)-5,5′-dimethoxy-[1,1′-biphenyl]-2-yl)oxy)-4H-naphtho[2,3-d][1,3,2]dioxaphosphinin-4-one, 2-((3,3′-di-tert-butyl-2′-((4,8-di-tert-butyl-2,10-dimethoxydibenzo[df][1,3,2]dioxaphosphepin-6-yl)oxy)-5,5′-dimethoxy-[1,1′-biphenyl]-2-yl)oxy)-8-methyl-4H-benzo[d][1,3,2]dioxaphosphinin-4-one, (1S,1'S)-(−)-(2,7-di-tert-butyl-9,9-dimethyl-9H-xanthene-4,5-diyl)bis((1-naphthyl) (phenyl)phosphine) or (1S,1'S)-(−)-(2,7-di-tert-butyl-9,9-dimethyl-9H-xanthene-4,5-diyl)bis((4-methylphenyl) (phenyl)phosphine), (6,6′-dimethoxy-[1,1′-biphenyl]-2,2′-diyl)bis(di(furan-2-yl)phosphane) and 3,9-bis(2,4-bis(2-phenylpropan-2-yl)phenoxy)-2,4,8,10-tetraoxa-3,9-diphosphaspiro[5.5]undecane.

According to a particular embodiment of the invention, the bidentate phosphorous ligand is a bidentate phosphite ligand. Particularly, the bidentate phosphite ligand may be selected from the group consisting of 2,2′-bis(dibenzo[d,f][1,3,2]dioxaphosphepin-6-yloxy)-1,1′-biphenyl, 6,6′-[(3,3′-di-tert-butyl-5,5′-dimethoxy-1,1′-biphenyl-2,2′-diyl)bis(oxy)]bis(di-benzo[d,f][1,3,2]dioxaphosphepin), 6,6′-[[3,3′,5,5′-tetrakis(1,1-dimethylethyl)-[1,1′-biphenyl]-2,2′-diyl]bis(oxy)]bis-dibenzo[d,f][1,3,2]dioxaphosphepine, (6,6′-dimethoxy-[1,1′-biphenyl]-2,2′-diyl)bis(di(furan-2-yl)phosphane) and 3,9-bis(2,4-bis(2-phenylpropan-2-yl)phenoxy)-2,4,8,10-tetraoxa-3,9-diphosphaspiro[5.5]undecane.

The phosphorous ligand can be added into the reaction medium of the invention's process in a large range of concentrations. As non-limiting examples, one can cite as phosphorous ligand concentration values those ranging from about 0.001 mol % to about 50 mol %, relative to the amount of the of substrate, preferably from 0.005 mol % to about 50 mol %, relative to the amount of the of substrate, preferably from about 0.005 mol % to about 15 mol %, relative to the amount of the of substrate. The optimum concentration of the phosphorous ligand will depend, as the person skilled in the art knows, on the nature of the latter, on the nature of the substrate, on the nature of the metal complex, on the reaction temperature as well as on the desired time of reaction.

According to any one of the above embodiments, carbon monoxide and hydrogen gas may be generated in situ by known methods by the person skilled in the art, e.g. from methyl formate, formic acid, or formaldehyde. The CO/H₂ gas volume ratio is comprised between 2/1 to 1/5, preferably between 1/1 to 1/5 or preferably between 2/1 to 1/2, preferably between 1.5/1 to 1/1.5 and more preferably the ratio is 1/1.

The reaction can be carried out in the presence or absence of a solvent. When a solvent is required or used for practical reasons, then any solvent current in such reaction type can be used for the purposes of the invention. Non-limiting examples include C₆₋₁₂ aromatic solvents such as toluene, 1,3-diisopropylbenzene, cumene or pseudocumene, or mixtures thereof, alcoholic solvents such as methanol, ethanol, 2-methylbutan-2-ol or mixtures thereof, hydrocarbon solvents such as cyclohexane, heptane or mixtures thereof, esteric solvent such as n-butyl acetate, iso-propyl acetate, ethyl acetate or ethereal solvents such as methyl tetrahydrofuran, tetrahydrofuran or mixtures thereof. The choice of the solvent is function of the nature of the substrate and/or catalyst and the person skilled in the art is well able to select the solvent most suitable in each case to optimize the reaction.

The hydroformylation reaction can be carried out at a temperature in the range comprised between 50° C. and 150° C., more preferably in the range comprised between 80° C. and 130° C., or even between 90° C. and 110° C. Of course, a person skilled in the art is also able to select the preferred temperature according to the melting and boiling point of the starting and final products as well as the desired time of reaction or conversion.

The hydroformylation can be carried out at a CO/H₂ pressure comprised between 1 bar and 50 bar, preferably in the range of between 10 bar and 50 bar, more preferably in the range of between 10 bar and 20 bar. Of course, a person skilled in the art is well able to adjust the pressure as a function of the catalyst load and of the dilution of the substrate in the solvent.

According to a particular embodiment of the invention, the hydroformylation is performed in the presence of a diol. When the hydroformylation is performed in the presence of a diol a dimer may be formed. The dimer is of formula

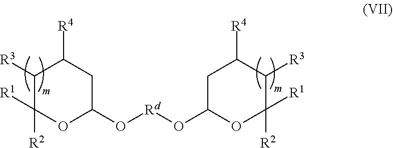

in the form of any one of its stereoisomers or a mixture thereof, and wherein R¹, R², R³, R⁴, R^{d} and m have the same meaning as defined above. Particularly, at most 20% of the dimer of formula (VII) is formed. The dimer may be converted into compound of formula (I) as defined above for compound of formula (II).

The compound of formula (II) is a novel compound and present a number of advantages as explained above and shown in the Examples. Therefore, another object of the present invention is a compound of formula

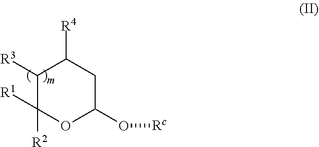

in the form of any one of its stereoisomers or a mixture thereof, and wherein m is 0 or 1; R¹ and R², independently from each other, represent a C₁₋₁₂ hydrocarbon group, optionally comprising one to three oxygen atoms and/or one to two nitrogen atoms and/or one sulfur atom; or R¹ and R² are taken together and form C₅₋₁₈ cycloalkyl or C₅₋₁₈ cycloalkenyl group, each optionally substituted by one or more hydroxy, C₁₋₁₅ alkyl, C₂₋₁₅ alkenyl, C₁₋₁₅ alkoxy, C₃₋₁₅ cycloalkyl, C₅₋₁₅ cycloalkenyl, C₁₋₆ ester, COOH, C₆₋₁₀ aryl and/or C₆₋₁₀ aryloxy groups; R³ and R⁴, independently from each other, represent a hydrogen atom or a C₁₋₆ alkyl group; and R^{c} is a hydrogen atom, a C₁₋₁₂ hydrocarbon group optionally comprising one to five oxygen atoms or a R^{c} is a group of formula

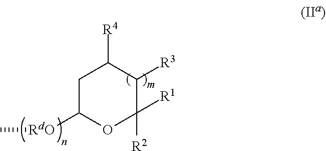

wherein R¹, R², R³, R⁴ and m have the same meaning as defined in formula (II); n is 0 or 1, R^{d} is a C₁₋₁₂ hydrocarbylene group optionally comprising one to five oxygen atoms and the hatched line indicates the location of the bond between (II^{a}) and the oxygen atom of formula (II); provided that 4-butyl-1-[(5,5-dibutyltetrahydro-2-furayl)oxy]-2,4-octanediol and 2-[(Tetrahydro-5,5-dimethyl-2-furanyl)oxy]ethanol are excluded.

Another object of the present invention is the use of a compound of formula

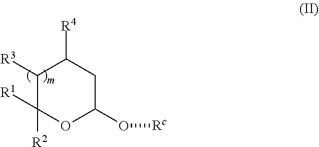

in the form of any one of its stereoisomers or a mixture thereof, and wherein m is 0 or 1; R¹ represents a C₁₋₁₂ hydrocarbon group, optionally comprising one to three oxygen atoms and/or one to two nitrogen atoms and/or one sulfur atom; R² represents a hydrogen atom or a R¹ group; or R¹ and R² are taken together and form C₅₋₁₈ cycloalkyl or C₅₋₁₈ cycloalkenyl group, each optionally substituted by one or more hydroxy, C₁₋₁₅ alkyl, C₂₋₁₅ alkenyl, C₁₋₁₅ alkoxy, C₃₋₁₅ cycloalkyl, C₅₋₁₅ cycloalkenyl, C₁₋₆ ester, COOH, C₆₋₁₀ aryl and/or C₆₋₁₀ aryloxy groups; R³ and R⁴, independently from each other, represent a hydrogen atom or a C₁₋₆ alkyl group; and R^{c} is a hydrogen atom, a C₁₋₁₂ hydrocarbon group optionally comprising one to five oxygen atoms or a R^{c} is a group of formula

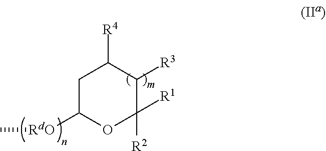

wherein R¹, R², R³, R⁴ and m have the same meaning as defined in formula (II); n is 0 or 1, R^{d} is a C₁₋₁₂ hydrocarbylene group optionally comprising one to five oxygen atoms and the hatched line indicates the location of the bond between (II^{a}) and the oxygen atom of formula (II);
in the preparation of a compound of formula (I).

Typical manners to execute the invention's process are reported herein below in the examples.

### EXAMPLES

The invention will now be described in further detail by way of the following examples, wherein the abbreviations have the usual meaning in the art, the temperatures are indicated in degrees centigrade (° C.). The preparation of precatalysts and ligands solutions were carried out under an inert atmosphere (Argon) using standard Schlenk techniques.

The solvents were dried by conventional procedures and distilled under an argon atmosphere. NMR spectra were recorded at 20° C. on Bruker AV 300, AV 400, or AV 500 MHz spectrometers. Chemical shifts are reported in ppm relative to solvent signals (chloroform, δ_{H}=7.26 ppm, δ_{C}=77.0 ppm). The signal assignment was ensured by recording ¹H,¹H— COSY, —NOESY, ¹³C,¹H—HSQC and —HMBC experiments. Gas chromatography was performed on an Agilent 7890 A Series equipped with a HP5 column (30 m×0.25 mm ID, 0.25 μm film) and tetradecane was used as internal standard.

### Example 1

### Hydroformylation of 4,4-dimethyl-1-vinyl-cyclohexan-1-ol

The autoclave was charged with 4,4-dimethyl-1-vinylcyclohexan-1-ol (74.38 g, 482.18 mmol), 1,2-ethanediol (101.24 g, 1.63 mol), 6,6′-[(3,3′-Di-tert-butyl-5,5′-dimethoxy-1,1′-biphenyl-2,2′-diyl)bis(oxy)]bis(dibenzo[d,f][1,3,2]dioxaphosphepin) [BiPhePhos](1.1245 mg, 0.0014 mmol) and [Rh(CO)₂acac](5×10⁻⁴ mmol [Rh]). The vessel was purged with H₂/CO (1:1, 4×5 and 2×10 bar and pressurized at 10 bar and heated 384 h at 75° C. under vigorous stirring to reach 97.6% conversion. After cooling and depressurization, GLC analysis of the slightly turbid reaction mixture revealed the presence of 4,4-dimethyl-1-vinyl-cyclohexan-1-ol (1.85%), 2-((8,8-dimethyl-1-oxaspiro[4.5]decan-2-yl)oxy)ethan-1-ol (76.4%), 8,8-dimethyl-1-oxaspiro[4.5]decan-2-ol (1.3%), 1,2-bis((8,8-dimethyl-1-oxaspiro[4.5]decan-2-yl)oxy)ethanes (12.7%) and hydrogenated starting material (4.1%). This crude reaction mixture (186.5 g) was further processed in the next stage.

### 2-((8,8-dimethyl-1-oxaspiro[4.5]decan-2-yl)oxy)ethan-1-ol

¹H-NMR (500.15 MHz, CDCl₃): 30.90 (s, 3H), 0.94 (s, 3H), 1.18-1.28 (m, 2H), 1.39-1.51 (m, 4H), 1.54-1.65 (m, 2H), 1.72-1.80 (m, 2H), 1.81-1.89 (m, 1H), 1.93-2.00 (m, 1H), 2.01-2.09 (m, 1H), 3.68-3.75 (m, 4H), 5.07-5.09 (m, 1H).

¹³C NMR (150 MHz, CDCl₃): δ 28.1 (q), 29.4 (s), 32.4 (t), 33.5 (t), 33.9 (t), 35.3 (t), 36.6(t), 62.6 (t), 70.9 (t), 84.8 (s), 104.8 (d).

### 1,2-bis((8,8-dimethyl-1-oxaspiro[4.5]decan-2-yl)oxy)ethanes

¹H-NMR (500.15 MHz, CDCl₃): δ 0.9 (s, 6H), 0.94 (s, 6H), 1.19-1.28 (m, 4H), 1.38-1.52, (m, 6H), 1.53-1.64 (m, 4H), 1.66-1.78 (m, 4H), 1.79-1.89 (m, 2H), 1.92-2.01 (m, 4H), 3.48-3.59 (m, 2H), 3.75-3.85 (m, 2H), 5.05-5.12 (m, 2H).

¹³C NMR (125 MHz, CDCl₃): 328.3 (q), 29.4 (s), 32.4 (t), 33.8 (t), 35.4 (t), 36.6 (t), 36.8 (t), 65.7 (t), 84.2 (s), 103.5 (d).

### Example 2

### Preparation of (2-(2-(4,4-dimethylcyclohex-1-en-1-yl)ethyl)-1,3-dioxolane from 2-((8,8-dimethyl-1-oxaspiro[4.5]decan-2-yl)oxy)ethan-1-ol and 1,2-bis((8,8-dimethyl-1-oxaspiro[4.5]decan-2-yl)oxy)ethane

186.5 g of a biphasic mixture of (2-(2-(4,4-dimethylcyclohex-1-en-1-yl)ethyl)-1,3-dioxolane and 1,2-bis((8,8-dimethyl-1-oxaspiro[4.5]decan-2-yl)oxy)ethane and 1,2-ethanediol were obtained from the hydroformylation reaction from 74.38 g (482.18 mmol) 4,4-dimethyl-1-vinylcyclohexan-1-ol, as reported in Example 1.

The 1,2-ethanediol phase (˜71.2 g) was separated from the product phase ˜115.3 g. The 1,2-ethanediol phase (˜71.2 g) was mixed with 45 g 1,2-ethanediol, 330 g xylene and 1.24 g (96%) H₂SO₄ (2.5 mol %) in a 1 L flask with a small column (2 elements Sulzer EX) and a Dean Stark apparatus. The mixture was dried and 115.3 g of product phase was added in 2.5 hours (internal temperature 130° C.-138° C.) and water was collected (Dean Stark apparatus). After 1.5 hours of further heating (no starting material) the mixture was cooled down and the 1,2-ethanediol phase was separated (99.6 g). The organic phase was washed with 160 g of a saturated aqueous NaHCO₃ solution and twice with 93 g water. Xylene was distilled of (75 mbar, 73° C.-110° C. internal temperature). The crude (103.6 g crude) was distilled through a Vigreux column (1.5 mbar, 145° C. internal temperature at the end). 78.85 g (374.9 mmol, 77.7% yield over 2 steps) 2-(2-(4,4-dimethylcyclohex-1-en-1-yl)ethyl)-1,3-dioxolane (and 3.8 g (18.1 mmol, 3.8% yield over 2 steps) 2-(2-(4,4-dimethylcyclohexylidene)ethyl)-1,3-dioxolane) were obtained.

### (2-(2-(4,4-dimethylcyclohex-1-en-1-yl)ethyl)-1,3-dioxolane

¹H-NMR (500.15 MHz): 0.88 (s, 6H), 1.35 (t, 2H, J=6.5 Hz), 1.74-1.79 (m, 4H), 1.92-1.97 (m, 2H), 2.07 (t, 2H, J=8.3 Hz), 3.82-3.88 (m, 2H), 3.93-4.00 (m, 2H), 4.86 (t, 1H, J=4.9 Hz), 5.34 (m, 1H).

¹³C NMR (125 MHz, CDCl₃): δ 26.2, 28.2, 28.5, 31.8, 32.2, 35.7, 39.3, 64.9, 104.5, 120.1, 135.4.

### 2-(2-(4,4-dimethylcyclohexylidene)ethyl)-1,3-dioxolane

¹H-NMR (500.15 MHz): 0.93 (s, 6H), 1.28-1.35 (m, 4H), 1.42 (s, 2H), 2.11-2.17 (m, 4H), 2.37-2.41 (m, 2H), 3.82-3.88 (m, 2H), 3.93-4.00 (m, 2H), 4.84 (t, 1H, J=4.9 Hz), 5.14 (t, 1H, J=7.2 Hz).

¹³C NMR (100 MHz, CDCl₃): δ 24.8, 28.2, 30.6, 32.3, 32.9, 40.1, 40.9, 64.9, 104.6, 114.0, 142.8.

The same yield for 2-(2-(4,4-dimethylcyclohex-1-en-1-yl)ethyl)-1,3-dioxolane (77% yield, 3.8% 2-(2-(4,4-dimethylcyclohexylidene)ethyl)-1,3-dioxolane) could be obtained by using pure 1,2-bis((8,8-dimethyl-1-oxaspiro[4.5]decan-2-yl)oxy)ethanes for the reaction (5 eq 1,2-ethanediol, H₂SO₄ (2.5 mol %), Xylene, azeotropic distillation of water).

### Example 3

### Preparation of 3-(4,4-dimethylcyclohex-1-en-1-yl)propanal from 2-(2-(4,4-dimethylcyclohex-1-en-1-yl)ethyl)-1,3-dioxolane

78.85 g (374.9 mmol) 2-(2-(4,4-dimethylcyclohex-1-en-1-yl)ethyl)-1,3-dioxolane (and 3.8 g (18.1 mmol) 2-(2-(4,4-dimethylcyclohexylidene)ethyl)-1,3-dioxolane), 83.5 g heptane, 125.3 g water and 125.3 g AcOH (5 eq) were mixed and heated under stirring at 80° C. for 4 hours (72% conversion). The mixture was cooled down to 0° C.-5° C. and a 36% aqueous NaOH solution (69.6 g NaOH, 139 g water) was added in 1.5 hours (internal temperature under 10° C.). The aqueous phase was separated (474.7 g, pH 6). The organic phase (157.3 g) is washed with 58.2 g of a saturated aqueous NaHCO₃ solution and 57.2 g water. Heptane (83.5 g) is distilled of (50-77° C., 190-30 mbar) and the mixture was distilled through a Vigreux column. 45.2 g (272.1 mmol) 3-(4,4-dimethylcyclohex-1-en-1-yl)propanal and 1.6 g (9.6 mmol) 3-(4,4-dimethylcyclohexylidene)propanal were obtained. Also 21.84 g (103.8 mmol) 2-(2-(4,4-dimethylcyclohex-1-en-1-yl)ethyl)-1,3-dioxolane (and 1.13 g (5.35 mmol) 2-(2-(4,4-dimethylcyclohexylidene)ethyl)-1,3-dioxolane) were recycled (yield on conversion of 99.2%).

### 3-(4,4-dimethylcyclohex-1-en-1-yl)propanal

¹H-NMR (300.13 MHz): 0.86 (s, 6H, 4′-(CH₃)₂), 1.34 (t, 2H, ³J_{5′,6′}=6.4 Hz, H-5′), 1.75 (m, 2H, H-3′), 1 h 0.88-1.94 (m, 2H, H-6′), 2.28 (m, 2H, H-3), 2.48-2.54 (m, 2H, H-2), 5.32 (m, 1H, H-2′), 9.74 (t, 1H, ²J_{1,2}=2.0 Hz, H-1).

¹³C-NMR (75.47 MHz): 26.2 (C-6′), 28.1 (CH₃), 28.4 (C-4′), 29.7 (C-3), 35.5 (C-5′), 39.1 (C-3′), 41.9 (C-2), 120.9 (C-2′), 134.2 (C-1′), 202.7 (C-1).

### 3-(4,4-dimethylcyclohexylidene)propanal

¹H-NMR (500.15 MHz): 0.95 (s, 6H), 1.32 (t, 2H, J=6.3 Hz), 1.36 (t, 2H, J=6.4 Hz), 2.12 (t, 2H, J=6.5 Hz), 2.17 (t, 2H, J=6.2 Hz), 3.13 (dd, 2H, J=7.3 Hz, J=1.0 Hz) 5.22 (t, 1H, J=7.3 Hz), 9.62 (t, 1H, J=2.1 Hz).

¹³C NMR (125 MHz, CDCl₃): δ 25.0, 28.1, 30.6, 32.8, 40.0, 40.8, 42.6, 109.5, 145.6, 200.2.

### Example 4

### Preparation of 2-methyl-1-(p-tolyl)but-3-en-2-ol from 4-methylphenylacetone

To a cooled solution (0° C.) of 139.9 mL vinylmagnesium chloride (1.6 M in THF, 254.4 mmol, 1.1 eq) was added slowly a solution of 1-(p-tolyl)propan-2-one (37.7 g, 254.4 mmol) in 153 mL THF. The internal temperature did not exceed 5° C. during the addition of the ketone. The mixture was further stirred at 0° C. over night (16 hours) and analysed by GC. The reaction mixture was added slowly to a cooled solution of 18.3 g AcOH (305.2 mmol) in 200 mL water. The phases were separated and the aqueous phase was extracted with 150 mL TBME. The combined organic phase were washed with a saturated aqueous NaHCO₃ solution and a saturated aqueous NaCl solution. After drying over Na₂SO₄ the solvent was evaporated under reduced pressure (500-20 mbar, 50° C.). The crude (48.2 g) was purified by a distillation through a Vigreux column under reduced pressure (oilbath 40° C.-125° C., 50-4 mbar, bp 97° C./4 mbar). 35.5 g (201.4 mmol, 79% yield) 2-methyl-1-(p-tolyl)but-3-en-2-ol of a colourless liquid were obtained.

### 2-methyl-1-(p-tolyl)but-3-en-2-ol

¹H-NMR analysis results in CDCl₃ were in accordance with data from literature:
Araki, S.; Ohmura, M.; Butsugan, Y. Bulletin of the Chemical Society of Japan (1986), 59(6), 2019-20.

¹³C NMR (100 MHz, CDCl₃): 321.0, 27.4, 48.3, 73.0, 111.9, 128.8, 130.5, 133.7, 136.1, 144.8.

### Example 5

### Preparation of (E)-2-(3-methyl-4-(p-tolyl)but-3-en-1-yl)-1,3-dioxolane from 2-((5-methyl-5-(4-methylbenzyl)tetrahydrofuran-2-yl)oxy)ethan-1-ol and 1,2-bis((5-methyl-5-(4-methylbenzyl)tetrahydrofuran-2-yl)oxy)ethane

The autoclave was charged with 2-methyl-1-(p-tolyl)but-3-en-2-ol (12.9 g, 73.2 mmol), 1,2-ethanediol (14.17 g, 0.228 mol), BiPhePhos solution (7.55×10⁻⁴ mmol) and Rh(CO)₂acac solution (2.46×10⁻⁴ mmol [Rh]). The vessel was purged with H₂/CO (1:1, 4×5 bar), pressurized at 10 bar and then heated under vigorous stirring at 90° C. and constant pressure for 24 h to reach >99% conversion. GLC analysis of the crude revealed the presence of 2-((5-methyl-5-(4-methylbenzyl)tetra-hydrofuran-2-yl)oxy)ethan-1-ol (47%, 33%), hydrogenated starting material (7%), and some minor unidentified products. Parts of this crude reaction mixture (27.0 g, contains 14.7 g of a mixture of 2-((5-methyl-5-(4-methylbenzyl)tetrahydrofuran-2-yl)oxy)ethan-1-ol and 1,2-bis((5-methyl-5-(4-methylbenzyl)tetrahydrofuran-2-yl)oxy)ethane, 80% yield, 58.6 mmol) were further processed in the next stage.

### 2-((5-methyl-5-(4-methylbenzyl)tetrahydrofuran-2-yl)oxy)ethan-1-ol

¹³C NMR (125 MHz, CDCl₃): 321.0 (q), 28.7 (q), 33.1 (t), 33.9 (t), 47.0 (t), 62.6 (t), 71.0 (t), 85.3 (s), 105.6 (d), 128.7 (d), 130.2 (d), 134.8 (s), 135.8 (s).

¹³C NMR (125 MHz, CDCl₃): 321.0 (q), 25.9 (q), 32.6 (t), 34.6 (t), 48.3 (t), 62.3 (t), 70.2 (t), 85.4 (s), 105.6 (d), 128.7 (d), 130.1 (d), 135.0 (s), 135.8 (s).

### 1,2-bis((5-methyl-5-(4-methylbenzyl) tetrahydrofuran-2-yl)oxy)ethane

¹H-NMR (500.15 MHz, CDCl₃): δ 1.33 (s, 3H), 1.34 (s, 3H), 1.37-1.47 (m, 2H), 1.73-1.84 (m, 6H), 2.31 (s, 6H), 2.63 (dd, 2H), 2.75 (dd, 2H), 3.5-3.56 (m, 2H), 3.76-3.83 (m, 2H), 4.99-5.0 (m, 2H), 7.04-7.13 (m, 6H).

¹³C NMR (125 MHz, CDCl₃): 321.0 (q), 29.1 (q), 33.1 (t), 33.5 (t), 47.2 (t), 65.8 (t), 84.8 (s), 104.3 (d), 128.6 (d), 130.3 (d), 135.2 (s), 135.6 (s) (signals of one stereo isomer).

In a 10 mL reactor (equipped with a Dean-Stark apparatus and a small column, 2 elements sulzer EX) 3.0 g (from 8.13 mmol 2-methyl-1-(p-tolyl)but-3-en-2-ol) of the mixture of (2-((5-methyl-5-(4-methylbenzyl)tetrahydrofuran-2-yl)oxy)ethan-1-ol and 1,2-bis((5-methyl-5-(4-methylbenzyl)tetrahydrofuran-2-yl)oxy)ethane) from the above hydroformylation reaction (6.5 mmol) was stirred with 5 mL Xylene, 2.5 eq 1,2-ethanediol (overall 5 eq) and 13.1 mg (0.133 mmol, 2.5 mol %) (98%) H₂SO₄. The mixture was heated to 130° C.-138° C.) and water was collected (Dean Stark apparatus). After 3 hours a full conversion of (2-((5-methyl-5-(4-methylbenzyl)tetrahydrofuran-2-yl)oxy)ethan-1-ol and 1,2-bis((5-methyl-5-(4-methylbenzyl)tetrahydrofuran-2-yl)oxy)ethane) was observed by GC. The mixture is diluted with 10 mL of MTBE and washed with 5 mL of water. The organic phase was then washed with 5 mL of a saturated aqueous NaHCO₃ solution, once with 5 mL of water, and with 5 mL of a saturated aqueous NaCl solution. After drying over sodium sulfate the solvent was evaporated under reduced pressure (50° C., 15 mbar). The crude was purified by column chromatography (80 g cartridge, from pentane 100% to MTBE 100%). 937 mg (4.035 mmol, 62% yield) of a mixture comprising (E)-2-(3-methyl-4-(p-tolyl)but-3-en-1-yl)-1,3-dioxolane, (Z)-2-(3-methyl-4-(p-tolyl)but-3-en-1-yl)-1,3-dioxolane, (E/Z)-2-(3-methyl-4-(p-tolyl)but-2-en-1-yl)-1,3-dioxolane and 2-(3-(4-methylbenzyl)but-3-en-1-yl)-1,3-dioxolane (ratio 38.5/31.1/25.4/4.0) was isolated.

### (E)-2-(3-methyl-4-(p-tolyl)but-3-en-1-yl)-1,3-dioxolane

¹H-NMR (500.15 MHz): 1.86 (s, 3H), 1.87-1.90 (m, 2H), 2.26-2.30 (m, 2H), 2.34 (s, 3H), 3.83-3.90 (m, 2H), 3.96-4.01 (m, 2H), 4.92 (t, 1H, J=4.7 Hz), 6.27 (s, 1H), 7.08-7.15 (m, 4H).

¹³C NMR (125 MHz, CDCl₃): δ 17.8, 21.1, 32.4, 34.9, 64.9, 104.2, 124.9, 128.7, 128.7, 135.5, 135.5, 137.3.

### (Z)-2-(3-methyl-4-(p-tolyl)but-3-en-1-yl)-1,3-dioxolane (Signals from the Mixture)

¹H-NMR (500.15 MHz): 1.88 (s, 3H), 1.84-1.86 (m, 2H), 2.32 (s, 3H), 2.34-2.38 (m, 2H), 3.80-3.86 (m, 2H), 3.92-3.98 (m, 2H), 4.87 (t, 1H, J=4.6 Hz), 6.26 (s, 1H), 7.11-7.15 (m, 4H).

¹³C NMR (125 MHz, CDCl₃): 321.1, 24.1, 26.9, 32.3, 64.9, 104.3, 125.9, 135.3, 135.5, 137.7

### (E)-2-(3-methyl-4-(p-tolyl)but-2-en-1-yl)-1,3-dioxolane (Characteristic Signals)

¹H-NMR (500.15 MHz): 2.31 (s, 3H), 2.41-2.45 (m, 2H), 3.28 (s, 2H).

¹³C NMR (125 MHz, CDCl₃): 3104.3, 119.28.

### (Z)-2-(3-methyl-4-(p-tolyl)but-2-en-1-yl)-1,3-dioxolane (Characteristic Signals)

¹H-NMR (500.15 MHz): 3.35 (s, 2H).

¹³C NMR (125 MHz, CDCl₃): 3104.3, 119.25.

### 2-(3-(4-methylbenzyl)but-3-en-1-yl)-1,3-dioxolane (Characteristic Signals)

¹H-NMR (500.15 MHz): 3.30 (s, 2H), 4.75 (s, 1H), 4.83 (s, 1H).

¹³C NMR (125 MHz, CDCl₃): 3104.2, 111.0, 148.3.

### Example 6

### Preparation of 2-methyl-1-(p-tolyl)but-3-en-1-ol from 4-methylbenzaldehyde

To a cooled solution (0° C.) of 494.4 mL 1-methyl-2-propenylmagnesium chloride (0.5 M in THF, 247.2 mmol, 1.1 eq) was added slowly a solution of 4-methylbenzaldehyde (27.0 g, 224.7 mmol) in 135 mL THF. The internal temperature did not exceed 5° C. during the addition of the aldehyde. The mixture was further stirred at 0° C. overnight (16 hours) and analysed by GC. The reaction mixture was added slowly to a cooled solution of 16.2 g AcOH (269.7 mmol) in 200 mL water. The phases were separated and the aqueous phase was extracted twice with 150 mL TBME. The combined organic phase were washed with a saturated aqueous NaHCO₃ solution and a saturated aqueous NaCl solution. After drying over Na₂SO₄ the solvent was evaporated under reduced pressure (500-4 mbar, 50° C.). The crude (44.3 g) was purified by a distillation through a Vigreux column under reduced pressure (oilbath 120° C., 900-3 mbar, bp 90° C./3 mbar). 39.0 g (221.3 mmol, 98.4% yield) 2-methyl-1-(p-tolyl)but-3-en-1-ol (syn/anti mixture) of a colourless liquid were obtained.

NMR analysis results in CDCl₃ were in accordance with data from literature for the syn isomer:
Shibata, I.; Yoshimura, N.; Yabu, M. Baba, A. Eur. J. Org. Chem. 2001, 3207-3211.
S. Hayashi, K. Hirano, H. Yorimitsu, K. Oshima Org. Lett. 2005, 7, 16, 3577-3579.

### 2-methyl-1-(p-tolyl)but-3-en-1-ol (Syn Isomer)

¹³C NMR (125 MHz, CDCl₃): δ 14.2, 21.1, 44.6, 77.2, 115.4, 126.5, 128.8, 137.0, 139.6, 140.4.

### 2-methyl-1-(p-tolyl)but-3-en-1-ol (Anti Isomer)

¹³C NMR (125 MHz, CDCl₃): 316.6, 21.1, 46.2, 77.7, 116.7, 126.8, 128.9, 137.3, 139.5, 140.8.

### Example 7

### Preparation of (E)-2-(3-methyl-4-(p-tolyl)but-3-en-1-yl)-1,3-dioxolane from 2-((5-methyl-6-(p-tolyl)tetrahydro-2H-pyran-2-yl)oxy)ethan-1-ol and 1,2-bis((5-methyl-6-(p-tolyl)tetrahydro-2H-pyran-2-yl)oxy)ethane

The autoclave was charged with 2-methyl-1-(p-tolyl)but-3-en-1-ol (2.02 g, 11.09 mmol), ethylene glycol (2.24 g, 36.09 mmol), Rh(CO)₂acac (1.6 mg, 0.0062 mmol) and BiPhePhos (13.6 mg, 0.0173 mmol). The vessel was purged with H₂/CO (1:1, 4×5 bar), pressurized at 10 bar and then heated under vigorous stirring at 90° C. and constant pressure for 24 h to reach >99% conversion. The crude reaction mixture was diluted with Et₂O (25 mL) and washed with water (2×5 mL). The organic phase was dried over anhydrous sodium sulfate, filtered and concentrated to give a light brown oil (2.15 g, 7.76 mmol, 70% estimated overall yield of 2-((5-methyl-6-(p-tolyl)tetrahydro-2H-pyran-2-yl)oxy)ethan-1-ols and 1,2-bis((5-methyl-6-(p-tolyl)tetrahydro-2H-pyran-2-yl)oxy)ethane), which was further processed in the next stage or purified by column chromatography.

### 2-((5-methyl-6-(p-tolyl)tetrahydro-2H-pyran-2-yl)oxy)ethan-1-ols

¹H-NMR (500.15 MHz): δ 0.6-0.94 (m, 4H), 1.14-2.03 (m, 5H), 2.19-2.41 (m, 4H), 3.35-4.00 (m, 4H), 4.21-5.07 (m, 1H), 7.1-7.24 (m, 4H).

¹³C NMR (150 MHz, CDCl₃): δ 11.09, 11.61, 12.34, 14.33, 17.14, 18.02, 20.25, 21.08, 21.17 (q), 24.15, 25.46, 26.01, 27.12, 27.38, 29.50, 30.32, 31.56, 31.6, 31.65 (t), 32.6, 32.81, 36.06, 36.16, 40.05 (d), 62.02, 62.22, 62.26, 62.32, 62.43, 69.13, 69.43, 69.49 (t), 72.0 (d), 72.18, 72.72 (t), 78.26, 79.81, 82.09, 85.1, 98.1, 98.46, 103.44, 104.23, 104.78, 125.27, 125.45, 126.26, 126.82, 127.21, 127.49, 128.69, 128.78, 128.85, 128.86, 128.87, 129.03, 129.05 (d), 136.13, 136.35, 137.04, 137.57, 137.66, 137.77, 137.78, 138.58 (s)

### 1,2-bis((5-methyl-6-(p-tolyl)tetrahydro-2H-pyran-2-yl)oxy)ethane

¹H-NMR (500.15 MHz): δ 0.53-2.07 (m, 14H), 2.14-2.47 (m, 8H), 3.54-3.80 (m, 4H), 4.91-5.10 (m, 4H), 7.07-7.24 (m, 8H).

¹³C NMR (125 MHz, CDCl₃): δ 11.2 (q), 21.1 (q), 24.1 (t), 25.6 (t), 32.7 (d), 65.9 (t), 71.4 (d), 97.8 (d), 125.5 (d), 128.6 (d), 135.9 (s), 139.1 (s).

In a 10 mL reactor (equipped with a Dean-Stark apparatus and a small column, 2 elements sulzer EX) 1.18 g (4.714 mmol) of 2-((5-methyl-6-(p-tolyl)tetrahydro-2H-pyran-2-yl)oxy)ethan-1-ol from the above hydroformylation reaction was stirred with 5 mL Xylene, 2.34 g (37.7 mmol) 1,2-ethanediol and 23.1 mg (0.133 mmol, 2.5 mol %) (98%) H₂SO₄. The mixture was heated to 130° C.-138° C.) and water was collected (Dean Stark apparatus). After 3 hours a full conversion of 2-((5-methyl-6-(p-tolyl)tetrahydro-2H-pyran-2-yl)oxy)ethan-1-ol was observed by GC. The mixture is diluted with 10 mL of MTBE and washed with 5 mL of water. The organic phase was then washed with 5 mL of a saturated aqueous NaHCO₃ solution, once with 5 mL of water, and with 5 mL of a saturated aqueous NaCl solution. After drying over sodium sulfate the solvent was evaporated under reduced pressure (50° C., 15 mbar). The crude was purified by column chromatography (80 g cartridge, from cyclohexane 100% to MTBE 100%). 470 mg (2.023 mmol, 42.9% yield) of a mixture of isomers (ratio 56.3/27.6/13.0/3.1) was isolated (1.139 mmol (E)-2-(3-methyl-4-(p-tolyl)but-3-en-1-yl)-1,3-dioxolane (56.3%) and 0.884 mmol of the other isomers (Z)-2-(3-methyl-4-(p-tolyl)but-3-en-1-yl)-1,3-dioxolane, (E/Z)-2-(3-methyl-4-(p-tolyl)but-2-en-1-yl)-1,3-dioxolane, 2-(3-(4-methylbenzyl)but-3-en-1-yl)-1,3-dioxolane, ratio 27.6/13.0/3.1).

### Example 8

### Preparation of (E)-4-methyl-5-(p-tolyl)pent-4-enal from (E)-2-(3-methyl-4-(p-tolyl)but-3-en-1-yl)-1,3-dioxolane

9.6 g (41.32 mmol) (E)-2-(3-methyl-4-(p-tolyl)but-3-en-1-yl)-1,3-dioxolane 14.5 mL heptane, 15 g water and 15 g AcOH (5 eq) were mixed and heated under stirring at 80° C. for 4 hours (63% conversion). The mixture was cooled down to 0° C.-5° C. and a 26% aqueous NaOH solution (9 g NaOH, 25 g water) was added in 1.5 hours (internal temperature under 10° C.). The aqueous phase was separated (pH 6) and reextracted with 15 mL heptane. The combined organic phases were washed with 10 mL water and 15 mL of a saturated aqueous NaHCO₃ solution. The organic phase was dried over Na₂SO₄ and the solvent was evaporated under reduced pressure. 8.73 g of a mixture of (E)-4-methyl-5-(p-tolyl)pent-4-enal (GC 63%, 29.2 mmol, 71% yield) and (E)-2-(3-methyl-4-(p-tolyl)but-3-en-1-yl)-1,3-dioxolane (GC 32%, 12.0 mmol, 29% yield) were obtained (99% yield on conversion).

### (E)-4-methyl-5-(p-tolyl)pent-4-enal

The ¹H and ¹³C NMR analysis results in CDCl₃ were in accordance with data from literature (see R. Moretti WO 2010052635 A1).

¹³C NMR (100 MHz, CDCl₃): δ 17.8, 21.1, 32.7, 42.2, 125.7, 128.7, 128.8, 135.1, 135.8, 136.0, 202.1.

The same deprotection can be done with (E/Z) mixtures of 2-(3-methyl-4-(p-tolyl)but-3-en-1-yl)-1,3-dioxolane (the same yield was achieved).

### (Z)-4-methyl-5-(p-tolyl)pent-4-enal (Signals from the Mixture)

¹³C NMR (100 MHz, CDCl₃): δ 21.1, 23.7, 24.9, 42.2, 126.9, 128.4, 128.9, 135.0, 135.9, 136.1, 202.0.

### Example 9

### Hydroformylation of 4,4-dimethyl-1-vinylcyclohexan-1-ol in 1,3-propanediol

The autoclave was charged with 4,4-dimethyl-1-vinylcyclohexan-1-ol (4.94 g, 32.415 mmol), 1,3-propanediol (8.49 g, 111.57 mmol), BiPhePhos (39.7 mg, 0.0505 mmol) and Rh(CO)₂acac (4.2 mg, 0.0163 mmol). The vessel was purged with H₂/CO 1:1 (4×5 bar) and heated under vigorous stirring at 90° C. and 10 bar syngas pressure for 72 h. After cooling and depressurization, the reaction mixture was diluted in Et₂O (20 ml) and washed with H₂O (20 ml). The aqueous layer was decanted and extracted with a second portion of Et₂O (20 ml). The reunified organic layers were dried with anhydrous sodium sulfate, filtered and concentrated to give a yellow oil (6.75 g), which was analyzed by GLC to reveal 99% conversion and the formation of hydrogenated starting material (6.6%), 8,8-dimethyl-1-oxaspiro[4.5]decan-2-ol (1.7%), 3-((8,8-dimethyl-1-oxaspiro[4.5]decan-2-yl)oxy)propan-1-ol (55.2%) and 1,3-bis((8,8-dimethyl-1-oxaspiro[4.5]decan-2-yl)oxy)propane (27.5%). Products were isolated by chromatography (Et₂O/pentane, 1:1) and identified by spectral means. The crude yellow oil remaining after the analyses (5.93 g) was further processed in the next step.

### 3-((8,8-dimethyl-1-oxaspiro[4.5]decan-2-yl)oxy)propan-1-ol

¹H-NMR (500.15 MHz, CDCl₃): δ 0.90 (s, 3H), 0.94 (s, 3H), 1.18-1.27 (m, 2H), 1.39-1.52 (m, 3H), 1.55-1.63 (m, 2H), 1.70-1.86 (m, 5H), 1.89-1.95 (m, 1H), 1.96-2.04 (m, 1H), 2.5 (s, br, 1H), 3.51-3.57 (m, 1H), 3.72-3.79 (m, 2H), 3.85-3.92 (m, 1H), 5.05 (d, 1H).

¹³C NMR (125 MHz, CDCl₃): δ 28.3 (q), 29.4 (s), 32.0 (t), 32.5 (t), 33.7 (t), 35.5 (t), 36.6 (t), 36.7 (t), 62.0 (t), 65.9 (t), 84.6 (s), 103.7 (d).

### 1,3-bis((8,8-dimethyl-1-oxaspiro[4.5]decan-2-yl)oxy)propane (Major Isomer)

¹H-NMR (500.15 MHz, CDCl₃): δ 0.90 (s, 3H), 0.94 (s, 3H), 1.18-1.27 (m, 4H), 1.38-1.52 (m, 6H), 1.53-1.61 (m, 4H), 1.67-1.76 (m, 4H), 1.77-1.85 (m, 4H), 1.88-1.99 (m, 4H), 3.36-3.45 (m, 2H), 3.69-3.79 (m, 2H), 5.04 (d, 2H).

¹³C NMR (125 MHz, CDCl₃): δ 28.3 (q), 29.4 (s), 30.1 (t), 32.4 (t), 33.8 (t), 35.5 (t), 36.6 (t), 36.7 (t), 63.7 (t), 84.1 (s), 103.5 (d).

### Example 10

### Preparation of 2-(2-(4,4-dimethylcyclohex-1-en-1-yl)ethyl)-1,3-dioxane from 3-((8,8-dimethyl-1-oxaspiro[4.5]decan-2-yl)oxy)propan-1-ol and 1,3-bis((8,8-dimethyl-1-oxaspiro[4.5]decan-2-yl)oxy)propane

In a 50 mL reactor (equipped with a Dean-Stark apparatus and a small column, 2 elements sulzer EX) 4.79 g of the crude of Example 9 (hydroformylation reaction, 22.529 mmol 4,4-dimethyl-1-vinylcyclohexan-1-ol) was stirred with 25 mL Xylene, 8.67 g (112.65 mmol) 1,3-propanediol and 55.2 mg (0.563 mmol, 2.5 mol %) (98%) H₂SO₄. The mixture was heated to 130° C.-138° C. and water was collected (Dean Stark apparatus). After 1.14 hours a full conversion of 3-((8,8-dimethyl-1-oxaspiro[4.5]decan-2-yl)oxy)propan-1-ol and 1,3-bis((8,8-dimethyl-1-oxaspiro[4.5]decan-2-yl)oxy)propane was observed by GC. After cooling down to room temperature the mixture was diluted with 20 mL of MTBE and washed with 10 mL of water. The organic phase was then washed with 10 mL of a saturated aqueous NaHCO₃ solution, once with 10 mL of water, and with 10 mL of a saturated aqueous NaCl solution. After drying over sodium sulfate the solvent was evaporated under reduced pressure (50° C., 15 mbar). The crude (4.22 g, GC 93.3%) was distilled (Kugelrohr). 3.77 g (16.81 mmol) 2-(2-(4,4-dimethylcyclohex-1-en-1-yl)ethyl)-1,3-dioxane and 0.201 g (0.896 mmol) 2-(2-(4,4-dimethylcyclohexylidene)ethyl)-1,3-dioxane) were obtained (78.6% yield over 2 steps).

### 2-(2-(4,4-dimethylcyclohex-1-en-1-yl)ethyl)-1,3-dioxane

¹H-NMR (500.15 MHz): 0.88 (s, 6H), 1.30-1.34 (m, 2H), 1.34 (t, 2H, J=6.4 Hz), 1.66-1.72 (m, 2H), 1.74-1.79 (m, 2H), 1.89-1.96 (m, 2H), 1.99-2.05 (m, 2H), 2.05-2.14, 3.71-3.79 (m, 2H), 4.07-4.13 (m, 2H), 4.49 (t, 1H, J=5.2 Hz), 5.30-5.34 (m, 1H).

¹³C NMR (125 MHz, CDCl₃): 325.9, 26.1, 28.2, 28.5, 31.7, 33.5, 35.7, 39.3, 66.9, 102.2, 120.0, 135.5.

### 2-(2-(4,4-dimethylcyclohexylidene)ethyl)-1,3-dioxane

¹³C NMR (125 MHz, CDCl₃) characteristic signal: 114.4.

### Example 11

### Preparation of 3-(4,4-dimethylcyclohex-1-en-1-yl)propanal from 2-(2-(4,4-dimethylcyclohex-1-en-1-yl)ethyl)-1,3-dioxane

2-(2-(4,4-dimethylcyclohex-1-en-1-yl)ethyl)-1,3-dioxane was transformed according the protocol of Example 3 to 3-(4,4-dimethylcyclohex-1-en-1-yl)propanal (80° C., 4.5 h). To increase the conversion the aqueous phase was separated and a fresh water/AcOH mixture was added and the mixture was further heated at 80° C. (1 h, 60% conversion).

### Example 12

### Hydroformylation of 4,4-dimethyl-1-vinylcyclohexan-1-ol in 1,2-pentanediol

The autoclave was charged with 4,4-dimethyl-1-vinylcyclohexan-1-ol (4.97 g, 32.609 mmol), 1,2-pentanediol (10.43 g, 100.15 mmol), BiPhePhos (38.7 mg, 0.0492 mmol) and Rh(CO)₂acac (4.3 mg, 0.0167 mmol). The vessel was purged with H₂/CO 1:1 (4×5 bar) and heated under vigorous stirring at 90° C. and 10 bar syngas pressure for 48 h. After cooling and depressurization, a sample (1.13 g) of the reaction mixture (15.36 g) was diluted in Et₂O (10 ml) and washed with H₂O (2×5 ml). The organic layer was dried with anhydrous sodium sulfate, filtered and concentrated to give a colorless oil (0.73 g), which was analyzed by GLC to reveal >99% conversion and the formation of hydrogenated starting material (5.6%), 8,8-dimethyl-1-oxaspiro[4.5]decan-2-ol (1.7%), 2-((8,8-dimethyl-1-oxaspiro[4.5]decan-2-yl)oxy)pentan-1-ol (10.1%), 1-((8,8-dimethyl-1-oxaspiro[4.5]decan-2-yl)oxy)pentan-2-ol (69.4%) and 2,2′-(pentane-1,2-diylbis(oxy))bis(8,8-dimethyl-1-oxaspiro[4.5]decanes) (2.4%). The products were isolated by chromatography (Et₂O/pentane, 1:1) and identified by spectral means. The crude reaction mixture remaining after the analyses (14.23 g) was further processed in the next step.

### 2-((8,8-dimethyl-1-oxaspiro[4.5]decan-2-yl)oxy)pentan-1-ol (Major Diastereomer)

¹H-NMR (500.15 MHz, CDCl₃): δ 0.87-0.96 (m, 9H), 1.16-1.36 (m, 4H), 1.36-1.49 (m, 5H), 1.55-1.61 (m, 1H), 1.62-1.69 (m, 1H), 1.74-1.83 (m, 2H), 1.84-1.91 (m, 1H), 1.95-2.0 (m, 1H), 2.05-2.13 (m, 1H), 3.43-3.58 (m, 3H), 4.32 (dd, 1H), 5.11 (dd, 1H).

¹³C NMR (150 MHz, CDCl₃): δ 14.0 (q), 18.9 (t), 28.3 (q), 29.4 (s), 32.5 (t), 33.3 (t), 34.1 (t), 35.0 (t), 36.5 (t), 36.6 (t), 66.6 (t), 83.3 (d), 85.0 (s), 105.5 (d).

### 1-((8,8-dimethyl-1-oxaspiro[4.5]decan-2-yl)oxy)pentan-2-ol (Isomer A)

¹H-NMR (500.15 MHz, CDCl₃): δ 0.85-0.97 (m, 9H), 1.17-1.29 (m, 2H), 1.32-1.51 (m, 7H), 1.54-1.65 (m, 2H), 1.72-1.88 (m, 3H), 1.93-1.99 (m, 1H), 2.0-2.1 (m, 1H), 3.35 (d, 1H), 3.42-3.47 (m, 1H), 3.61 (dd, 1H), 3.72-3.79 (m, 1H), 5.08 (dd, 1H).

¹³C NMR (125 MHz, CDCl₃): δ 14.1 (q), 18.8 (t), 28.2 (q), 29.4 (s), 32.5 (t), 33.5 (t), 33.9 (t), 35.1 (t), 35.3 (t), 36.5 (t), 36.6 (t), 70.4 (d), 74.0 (t), 84.9 (s), 104.8 (d).

### 1-((8,8-dimethyl-1-oxaspiro[4.5]decan-2-yl)oxy)pentan-2-ol (Isomer B)

¹H-NMR (500.15 MHz, CDCl₃): δ 0.86-0.96 (m, 9H), 1.23-1.28 (m, 4H), 1.34-1.52 (m, 5H) 1.55-1.63 (m, 2H), 1.71-1.78 (m, 2H), 1.79-1.86 (m, 1H), 1.95-2.06 (m, 2H), 2.67 (d, 1H), 3.31 (q, 1H), 3.68-3.77 (m, 2H), 5.07 (d, 1H).

¹³C NMR (150 MHz, CDCl₃): δ 14.1 (q), 18.8 (t), 28.2 (q), 29.4 (s), 32.4 (t), 33.6 (t), 33.8 (t), 35.4 (t), 35.4 (t), 36.6 (t), 36.7 (t), 70.3 (d), 72.6 (t), 84.8 (s), 104.7 (d).

### 2,2′-(pentane-1,2-diylbis(oxy))bis(8,8-dimethyl-1-oxaspiro[4.5]decanes) (Mixture of 4 Diastereomers)

¹H-NMR (500.15 MHz, CDCl₃): δ 0.86-0.97 (m, 15H), 1.16-2.02 (m, 28H), 3.23-3.84 (m, 3H), 5.01-5.42 (m, 2H).

¹³C NMR (150 MHz, CDCl₃): δ 14.21, 14.23, 14.27, 14.33 (q), 18.39, 18.63, 18.72, 18.82 (t), 28.23 (q, br), 29.38, 29.39, 29.41, 29.43 (s), 32.37, 32.39, 32.41, 32.46, 32.52, 32.56, 33.61, 33.68, 33.72, 33.75, 33.79, 33.84, 34.81, 34.83, 35.33, 35.37, 35.45, 36.66, 36.74, 36.81, 68.57, 68.63, 69.14, 69.25 (t), 73.77, 74.20, 75.02, 75.68 (d), 84.15 (s, br), 102.07, 102.36, 102.64, 103.4, 103.64, 103.77, 103.78, 103.9 (d).

### Example 13

### Preparation of 2-(2-(4,4-dimethylcyclohex-1-en-1-yl)ethyl)-4-propyl-1,3-dioxolane from 1-((8,8-dimethyl-1-oxaspiro[4.5]decan-2-yl)oxy)pentan-2-ol, 2-((8,8-dimethyl-1-oxaspiro[4.5]decan-2-yl)oxy)pentan-1-ol and 2,2′-(pentane-1,2-diylbis(oxy))bis(8,8-dimethyl-1-oxaspiro[4.5]decane)

In a 100 mL reactor (equipped with a Dean-Stark apparatus and a small column, 2 elements sulzer EX) 13.1 g of the crude of the Example 12 (hydroformylation reaction, 27.788 mmol 4,4-dimethyl-1-vinylcyclohexan-1-ol) was stirred with 50 mL Xylene, 7.235 g (69.47 mmol) 1,2-pentanediol and 61.3 mg (0.625 mmol, 2.2 mol %) (98%) H₂SO₄. The mixture was heated to 130° C.-138° C. and water was collected (Dean Stark apparatus). After 3 hours 40 min a full conversion of 1-((8,8-dimethyl-1-oxaspiro[4.5]decan-2-yl)oxy)pentan-2-ol, 2-((8,8-dimethyl-1-oxaspiro[4.5]decan-2-yl)oxy)pentan-1-ol and 2,2′-(pentane-1,2-diylbis(oxy))bis(8,8-dimethyl-1-oxaspiro[4.5]decane) was observed by GC. After cooling down to room temperature the mixture was diluted with 20 mL of MTBE and washed with 10 mL of water. The organic phase was then washed with 10 mL of a saturated aqueous NaHCO₃ solution, once with 10 mL of water, and with 10 mL of a saturated aqueous NaCl solution. After drying over sodium sulfate the solvent was evaporated under reduced pressure (50° C., 15 mbar). The crude (6.7 g) was distilled (Kugelrohr) for the purification. 5.64 g (22.37 mmol) 2-(2-(4,4-dimethylcyclohex-1-en-1-yl)ethyl)-4-propyl-1,3-dioxolane were obtained (80.5% yield over 2 steps). 2% of 2-(2-(4,4-dimethylcyclohexylidene)ethyl)-4-propyl-1,3-dioxolane were identified by GC/NMR.

¹H-NMR (500.15 MHz): 0.88 (s, 6H), 0.94 (t, 3H, J=7.2 Hz), 1.35 (t, 2H, J=6.5 Hz), 1.38-1.54 (m, 2.5H), 1.58-1.68 (m, 1.5H), 1.70-1.79 (m, 4H), 1.90-1.96 (m, 2H), 2.02-2.09 (m, 2H), 3.40-3.49 (m, 1H), 3.89-3.94 (m, 0.5H), 4.00-4.13 (m, 1.5H), 3.71-3.79 (m, 2H), 4.07-4.13 (m, 2H), 4.88 (t, 0.5H, J=4.8 Hz), 4.96 (t, 0.5H, J=4.9 Hz), 5.31-5.36 (m, 1H).

¹³C NMR (125 MHz, CDCl₃) major isomer: δ 14.1, 19.0, 26.2, 28.2, 28.2, 28.5, 31.7, 32.5, 35.6, 35.7, 39.3, 69.6, 76.6, 104.4, 119.9, 135.5.

¹³C NMR (125 MHz, CDCl₃) minor isomer: δ 14.1, 19.0, 26.2, 28.2, 28.2, 28.5, 31.8, 32.6, 35.3, 35.4, 39.3, 70.5, 75.8, 103.7, 120.0, 135.5.

### Example 14

### Preparation of 3-(4,4-dimethylcyclohex-1-en-1-yl)propanal from 2-(2-(4.4-dimethylcyclohex-1-en-1-yl)ethyl)-4-propyl-1,3-dioxolane

2-(2-(4,4-dimethylcyclohex-1-en-1-yl)ethyl)-4-propyl-1,3-dioxolane was transformed according the protocol of Example 3 to 3-(4,4-dimethylcyclohex-1-en-1-yl)propanal (80° C., 4.5 h). To increase the conversion the aqueous phase was separated and a fresh water/AcOH mixture was added and the mixture was further heated at 80° C.

### Example 15

### Hydroformylation of 4,4-dimethyl-1-vinylcyclohexan-1-ol in trans-1,2-cyclohexanediol

The autoclave was charged with 4,4-dimethyl-1-vinylcyclohexan-1-ol (5.00 g, 32.804 mmol), trans-1,2-cyclohexanediol (11.78 g, 99.384 mmol). This mixture was stirred half an hour to give a very viscous biphasic suspension. Then, BiPhePhos (39.8 mg, 0.0506 mmol) and Rh(CO)₂acac (4.1 mg, 0.0159 mmol) were added. The vessel was purged with H₂/CO 1:1 (4×5 bar) and heated under vigorous stirring at 90° C. and 10 bar syngas pressure for 48 h. After cooling and depressurization, the crude white solid reaction mass was dissolved with MeOH (5 ml) and Et₂O (20 ml). The resulting solution was washed with H₂O (3×20 ml) before the organic layer was dried with anhydrous sodium sulfate, filtered and concentrated to give a crude oil (7.97 g), which was analyzed by GLC to reveal total conversion and the formation of hydrogenated starting material (7.9%), 8,8-dimethyl-1-oxaspiro[4.5]decan-2-ol (5.9%), 2,2′-oxybis(8,8-dimethyl-1-oxaspiro[4.5]decane) (1.7%), 2-((8,8-dimethyl-1-oxaspiro[4.5]decan-2-yl)oxy)cyclohexan-1-ols (84%) and traces of the 1,2-bis((8,8-dimethyl-1-oxaspiro[4.5]decan-2-yl)oxy)cyclo-hexane. The products were isolated by chromatography (Et₂O/pentane, 1:1) and identified by spectral means. The crude reaction mixture remaining after the analyses (6.88 g) was further processed in the next step. 2-((8,8-dimethyl-1-oxaspiro[4.5]decan-2-yl)oxy)cyclohexan-1-ol (diastereomer A) ¹H-NMR (500.15 MHz, CDCl₃): δ 0.89 (s, 3H), 0.93 (s, 3H), 1.14-1.30 (m, 6H), 1.36-1.52 (m, 3H), 1.54-1.62 (m, 1H), 1.62-1.71 (m, 3H), 1.72-1.99 (m, 5H), 2.01-2.15 (m, 2H), 3.17-3.25 (m, 1H), 3.3-3.39 (m, 1H), 4.85 (s, 1H), 5.18 (dd, 1H).

¹³C NMR (125 MHz, CDCl₃): δ 23.9 (t), 24.5 (t), 28.1 (q), 29.4 (s), 31.6 (t), 32.5 (t), 32.8 (t), 33.3 (t), 34.2 (t), 35.0 (t), 36.4 (t), 36.6 (t), 73.7 (d), 85.0 (s), 85.4 (d), 104.6 (d).

### 2-((8,8-dimethyl-1-oxaspiro[4.5]decan-2-yl)oxy)cyclohexan-1-ol (Diastereomer B)

¹³C NMR (125 MHz, CDCl₃): δ 24.07 (t), 24.55 (t), 28.2 (q), 29.4 (s), 32.0 (t), 32.3 (t), 32.7 (t), 33.6 (t), 33.75 (t), 35.42 (t), 36.6 (t), 36.7 (t), 73.9 (d), 83.5 (d), 84.5 (s), 104.8 (d).

### Example 16

### Preparation of 2-(2-(4,4-dimethylcyclohex-1-en-1-yl)ethyl)hexahydrobenzo[d][1,3]dioxole from 2-((8,8-dimethyl-1-oxaspiro[4.5]decan-2-yl)oxy)cyclohexan-1-ol and 1,2-bis((8,8-dimethyl-1-oxaspiro[4.5]decan-2-yl)oxy)cyclohexane

In a 50 mL reactor (equipped with a Dean-Stark apparatus and a small column, 2 elements sulzer EX) 2 g of the crude of the Example 15 (hydroformylation reaction, 10.0 mmol 4,4-dimethyl-1-vinylcyclohexan-1-ol) was stirred with 20 mL Xylene, 2.96 g (25 mmol) trans-1,2-cyclohexanediol and 24.5 mg (0.25 mmol, 2.5 mol %) (98%) H₂SO₄. The mixture was heated to 130° C.-138° C. and water was collected (Dean Stark apparatus). After 3 hours 40 min an incomplete conversion of 2-((8,8-dimethyl-1-oxaspiro[4.5]decan-2-yl)oxy)cyclohexan-1-ol was observed by GC. After cooling down to room temperature the mixture was diluted with 20 mL of MTBE and washed with 10 mL of water. The organic phase was then washed with 10 mL of a saturated aqueous NaHCO₃ solution, once with 10 mL of water, and with 10 mL of a saturated aqueous NaCl solution. After drying over sodium sulfate the solvent was evaporated under reduced pressure (50° C., 15 mbar). The crude (1.6 g) was purified by column chromatography (80 g cartridge, from cyclohexane/MTBE 99/1 to cyclohexane/MTBE) and 2-(2-(4,4-dimethylcyclohex-1-en-1-yl)ethyl)hexahydrobenzo[d][1,3]dioxole was isolated (226 mg, 0.85 mmol, 8.5% yield). 1% 2-(2-(4,4-dimethylcyclohexylidene)ethyl)hexahydrobenzo[d][1,3]dioxole were identified by GC/NMR.

### 2-(2-(4,4-dimethylcyclohex-1-en-1-yl)ethyl)hexahydrobenzo[d][1,3]dioxole

¹H-NMR (500.15 MHz) 3 isomers: 0.88 (s, 6H), 1.24-1.31 (m, 2H), 1.35 (t, 2H, J=6.5 Hz), 1.39-1.51 (m, 4H), 1.74-1.84 (m, 4H) 1.90-1.96 (m, 2H), 2.06-2.17 (m, 4H), 3.15-3.26 (m, 2H), 5.15 (t, 1H, J=4.9 Hz), 5.33-5.37 (m, 1H).

¹³C NMR (150 MHz, CDCl₃): 323.7, 23.8, 26.2, 28.2, 28.5, 28.6, 29.0, 31.8, 33.5, 35.7, 39.3, 79.6, 81.8, 104.7, 119.9, 135.4.

### 2-(2-(4,4-dimethylcyclohexylidene)ethyl)hexahydrobenzo[d][1,3]dioxole

¹³C NMR (150 MHz, CDCl₃) characteristic signal: 3114.2.

### Example 17

### Hydroformylation of 4,4-dimethyl-1-vinylcyclohexan-1-ol in 2,3-butanediol

The autoclave was charged with 4,4-dimethyl-1-vinylcyclohexan-1-ol (4.98 g, 32.67 mmol), 2,3-butanediol (8.99 g, 99.755 mmol), BiPhePhos (39.3 mg, 0.05 mmol) and Rh(CO)₂acac (4.4 mg, 0.0171 mmol). The vessel was purged with H₂/CO 1:1 (4×5 bar) and heated under vigorous stirring at 90° C. and 10 bar syngas pressure for 48 h. After cooling and depressurization, a sample (1 g) of the reaction mixture (13.33 g) was diluted in Et₂O (10 ml) and washed with H₂O (10 ml). The aqueous layer was decanted and extracted with a second portion of Et₂O (10 ml). The combined organic layers were dried with anhydrous sodium sulfate, filtered and concentrated to give a pale yellow oil (0.62 g), which was analyzed by GLC to reveal >99% conversion and the formation of hydrogenated starting material (5.3%), 8,8-dimethyl-1-oxaspiro[4.5]decan-2-ol (4%), 3-((8,8-dimethyl-1-oxaspiro[4.5]decan-2-yl)oxy)butan-2-ol (67%) and 2,2′-oxybis(8,8-dimethyl-1-oxaspiro[4.5]decane) (1.1%). The products were isolated by chromatography (Et₂O/pentane, 1:1) and identified by spectral means. The crude reaction mixture remaining after the analyses (12.33 g) was further processed in the next step.

### 3-((8,8-dimethyl-1-oxaspiro[4.5]decan-2-yl)oxy)butan-2-ol (Major Isomer)

¹H-NMR (500.15 MHz, CDCl₃): δ 0.90 (s, 3H), 0.94 (s, 3H), 1.10 (d, 3H), 1.14 (d, 3H), 1.15-2.04 (m, 12H), 2.28 (s, br, 1H), 3.72 (m, 1H), 3.88 (m, 1H), 5.24 (dd, 1H).

¹³C NMR (125 MHz, CDCl₃): δ 14.6 (q), 17.7 (q), 28.2 (q), 29.4 (s), 32.6 (t), 33.7 (t), 35.5 (t), 36.6 (t), 68.7 (d), 76.1 (d), 84.5 (s), 102.3 (d).

### Example 18

### Preparation of 2-(2-(4,4-dimethylcyclohex-1-en-1-yl)ethyl)-4,5-dimethyl-1,3-dioxolane from 3-((8,8-dimethyl-1-oxaspiro[4.5]decan-2-yl)oxy)butan-2-ol

In a 50 mL reactor (equipped with a Dean-Stark apparatus and a small column, 2 elements sulzer EX) 4.0 g of the crude of the Example 17 (hydroformylation reaction, 9.68 mmol 4,4-dimethyl-1-vinylcyclohexan-1-ol) was stirred with 17 mL Xylene, 2.2 g (24.2 mmol) 2,3-butanediol and 23.8 mg (0.242 mmol, 2.5 mol %) (98%) H₂SO₄. The mixture was heated to 130° C.-138° C. and water was collected (Dean Stark apparatus). After 1 hour a full conversion of 3-((8,8-dimethyl-1-oxaspiro[4.5]decan-2-yl)oxy)butan-2-ol and 2,2′-(butane-2,3-diylbis(oxy))bis(8,8-dimethyl-1-oxaspiro[4.5]decane) was observed by GC. After cooling down to room temperature the mixture was diluted with 20 mL of MTBE and washed with 10 mL of water. The organic phase was then washed with 10 mL of a saturated aqueous NaHCO₃ solution, once with 10 mL of water, and with 10 mL of a saturated aqueous NaCl solution. After drying over sodium sulfate the solvent was evaporated under reduced pressure (50° C., 15 mbar). The crude (2.02 g) was distilled (Kugelrohr) for the purification. 1.83 g (7.659 mmol) 2-(2-(4,4-dimethylcyclohex-1-en-1-yl)ethyl)-4,5-dimethyl-1,3-dioxolane were obtained (79% yield over 2 steps). 2% 2-(2-(4,4-dimethylcyclohexylidene)ethyl)-4,5-dimethyl-1,3-dioxolane were identified by GC/NMR.

### 2-(2-(4,4-dimethylcyclohex-1-en-1-yl)ethyl)-4,5-dimethyl-1,3-dioxolane

¹H-NMR (500.15 MHz) 3 isomers: 0.88 (s, 6H), 0.94 (t, 3H, J=7.2 Hz), 1.11-1.16 (m, 6H), 1.35 (t, 2H, J=6.5 Hz), 1.71-1.78 (m, 4H) 1.90-1.96 (m, 2H), 2.00-2.10 (m, 2H), 3.55-3.63 (m, 0.6H), 4.07-4.14 (m, 1H), 4.18-4.26 (m, 0.4H), 4.86 (t, 0.5H, J=4.8 Hz), 5.03 (t, 0.3H, J=4.9 Hz), 5.17 (t, 0.2H, J=4.9 Hz), 5.31-5.36 (m, 1H).

¹³C NMR (125 MHz, CDCl₃): 315.5, 26.2, 28.2, 28.5, 31.8, 33.0, 35.7, 39.3, 74.4, 103.1, 119.9, 135.5.

¹³C NMR (125 MHz, CDCl₃) characteristic signals minor isomer: δ 74.2, 78.1, 79.7, 102.4, 103.2.

### 2-(2-(4,4-dimethylcyclohexylidene)ethyl)-4,5-dimethyl-1,3-dioxolane

¹³C NMR (125 MHz, CDCl₃) characteristic signals: 114.2, 114.3, 114.4.

### Example 19

### Preparation of 3-(4,4-dimethylcyclohex-1-en-1-yl)propanal from 2-(2-(4.4-dimethylcyclohex-1-en-1-yl)ethyl)-4,5-dimethyl-1,3-dioxolane

2-(2-(4,4-dimethylcyclohex-1-en-1-yl)ethyl)-4,5-dimethyl-1,3-dioxolane was transformed according the protocol of Example 3 to 3-(4,4-dimethylcyclohex-1-en-1-yl)propanal (80° C., 4.5 h). To increase the conversion the aqueous phase was separated and a fresh water/AcOH mixture was added and the mixture was further heated at 80° C.

### Example 20

### Hydroformylation of 4,4-dimethyl-1-vinylcyclohexan-1-ol in MeOH

The autoclave was charged with 4,4-dimethyl-1-vinylcyclohexan-1-ol (497.13 g, 3.26 mol), MeOH (365.94 g, 11.41 mol), BiPhePhos-solution (4 ml, 15.52 mg, 0.0197 mmol in 4,4-dimethyl-1-vinylcyclohexan-1-ol, 3.542 g, 22.9601 mmol) and Rh(CO)₂acac-solution (3.2 ml, 1.67 mg, 0.0065 mmol in 4,4-dimethyl-1-vinylcyclohexan-1-ol, 2.846 g, 18.4506 mmol). The vessel was purged with nitrogen (3×5 bar without stirring) and then with H₂/CO 1:1 (5×10 bar, without stirring). The reactor was pressurized at 10 bar and the mixture heated at 90° C. under vigorous stirring for 48 h at constant pressure. After cooling and depressurization, the crude was analyzed by GLC to reveal 96.6% conversion and the formation of hydrogenated starting material (2.6%), 8,8-dimethyl-1-oxaspiro[4.5]decan-2-ol (1.4%), 2,2′-oxybis (8,8-dimethyl-1-oxaspiro[4.5]decane) (0.4%) and 2-methoxy-8,8-dimethyl-1-oxaspiro[4.5]decane (92%). In order to completely convert the remaining starting material, BiPhePhos-solution (2 ml, 7.76 mg, 0.0099 mmol in 4,4-dimethyl-1-vinylcyclo-hexan-1-ol, 1.77 g, 11.4749 mmol) and [Rh(CO)₂acac-solution (1.5 ml, 0.78 mg, 0.003 mmol in 4,4-dimethyl-1-vinylcyclohexan-1-ol, 1.33 g, 8.6224 mmol) were injected and the hydroformylation continued at 6.5 bar for 21 h at 90° C. After cooling and depressurization, the crude emulsion (≈955.16 g) was analyzed by GLC to reveal total conversion (>99.5%) and the formation of hydrogenated starting material (2.6%), 8,8-dimethyl-1-oxaspiro[4.5]decan-2-ol (1.3%), 2,2′-oxybis (8,8-dimethyl-1-oxaspiro[4.5]decane) (0.3%) and 2-methoxy-8,8-dimethyl-1-oxaspiro[4.5]decane (95.3%). To isolate the main product a sample of the crude reaction mixture (250 mg) was diluted in Et₂O (5 ml) and washed with H₂O (2 ml). The aqueous layer was decanted, the organic solution dried with anhydrous sodium sulfate, filtered and concentrated to give almost pure 2-methoxy-8,8-dimethyl-1-oxaspiro[4.5]decane (93 mg) ready for spectral analysis. The remaining 952.96 g of crude reaction mixture (2 phases) were processed in the next step.

### 2-methoxy-8,8-dimethyl-1-oxaspiro[4.5]decane

¹H-NMR (500.15 MHz, CDCl₃): δ 0.90 (s, 3H), 0.95 (s, 3H), 1.19-1.27 (m, 2H), 1.39-1.53 (m, 3H), 1.55-1.62 (m, 2H), 1.69-1.85 (m, 3H), 1.89-2.02 (m, 2H), 3.32 (s, 3H), 4.96 (dd, 1H).

¹³C NMR (125 MHz, CDCl₃): 328.3 (q), 29.4 (s), 32.4 (t), 33.8 (t), 35.6 (t), 36.6 (t), 36.7 (t), 54.2 (q), 84.3 (s), 104.7 (d).

### Example 21

### Preparation of 2-(2-(4,4-dimethylcyclohex-1-en-1-yl)ethyl)-5,5-dimethyl-1,3-dioxane from 2-methoxy-8,8-dimethyl-1-oxaspiro[4.5]decane

In a 50 mL reactor (equipped with a Dean-Stark apparatus and a small column, 2 elements sulzer EX) 3.0 g of the crude of Example 20 (from 10.308 mmol 4,4-dimethyl-1-vinylcyclohexan-1-ol) were mixed after MeOH evaporation with 20 mL toluene, 5.37 g (51.54 mmol) 2,2-dimethyl-1,3-propanediol and 25.3 mg (0.258 mmol, 2.5 mol %) (98%) H₂SO₄. After stirring at 50° C. the formation of 3-((8,8-dimethyl-1-oxaspiro[4.5]decan-2-yl)oxy)-2,2-dimethylpropan-1-ol and 2,2′-((2,2-dimethylpropane-1,3-diyl)bis(oxy))bis(8,8-dimethyl-1-oxaspiro[4.5]decane) was observed by GC and GC-MS (30 min). The mixture was heated to reflux and water was collected (Dean Stark apparatus). After 1 hour a full conversion of 3-((8,8-dimethyl-1-oxaspiro[4.5]decan-2-yl)oxy)-2,2-dimethylpropan-1-ol and 2,2′-((2,2-dimethylpropane-1,3-diyl)bis(oxy))bis(8,8-dimethyl-1-oxaspiro[4.5]decane) was observed by GC. After cooling down to room temperature the mixture was diluted with 20 mL of MTBE and washed with 10 mL of water. The organic phase was then washed with 10 mL of a saturated aqueous NaHCO₃ solution, once with 10 mL of water, and with 10 mL of a saturated aqueous NaCl solution. After drying over sodium sulfate the solvent was evaporated under reduced pressure (50° C., 15 mbar). The crude (2.51 g) was distilled (Kugelrohr) for the purification. 2.245 g (8.895 mmol) 2-(2-(4,4-dimethylcyclohex-1-en-1-yl)ethyl)-5,5-dimethyl-1,3-dioxane and 88 mg (0.348 mmol) 2-(2-(4,4-dimethylcyclohexylidene)ethyl)-5,5-dimethyl-1,3-dioxane) were obtained (86% yield over 2 steps). 2-(2-(4,4-dimethylcyclohex-1-en-1-yl)ethyl)-5,5-dimethyl-1,3-dioxane

¹H-NMR (500.15 MHz): 0.71 (s, 3H), 0.88 (s, 6H), 1.19 (s, 3H), 1.35 (t, 2H, J=6.5 Hz), 1.71-1.78 (m, 4H), 1.90-1.96 (m, 2H), 2.02-2.08 (m, 1H), 3.39-3.43 (m, 2H), 3.58-3.61 (m, 2H), 4.39 (t, 1H, J=5.1 Hz), 5.31-5.35 (m, 1H).

¹³C NMR (125 MHz, CDCl₃): 321.9, 23.0, 26.2, 28.2, 28.5, 30.2, 31.7, 33.1, 35.7, 39.3, 77.3, 102.1, 120.0, 135.5.

### 2-(2-(4,4-dimethylcyclohexylidene)ethyl)-5,5-dimethyl-1,3-dioxane

¹³C NMR (125 MHz, CDCl₃) characteristic signal: 114.45.

The product obtained was transformed according the protocol of Example 3 to 3-(4,4-dimethylcyclohex-1-en-1-yl)propanal (80° C., 4-6 h). To increase the conversion the aqueous phase was separated and a fresh water/AcOH mixture was added and the mixture was further heated at 80° C. The procedure could be repeated several times.

### Example 22

### Preparation of 2-(2-(4,4-dimethylcyclohex-1-en-1-yl)ethyl)-5,5-dimethyl-1,3-dioxane from 2-methoxy-8,8-dimethyl-1-oxaspiro[4.5]decane

In a 50 mL reactor (equipped with a Dean-Stark apparatus and a small column, 2 elements sulzer EX) 3.0 g of the crude of Example 20 (from 10.308 mmol 4,4-dimethyl-1-vinylcyclohexan-1-ol) were mixed after MeOH evaporation with 20 mL toluene, 1.61 g (15.46 mmol) 2,2-dimethyl-1,3-propanediol and 25.3 mg (0.258 mmol, 2.5 mol %) (98%) H₂SO₄. After stirring at 50° C. the formation of 3-((8,8-dimethyl-1-oxaspiro[4.5]decan-2-yl)oxy)-2,2-dimethylpropan-1-ol and 2,2′-((2,2-dimethylpropane-1,3-diyl)bis(oxy))bis(8,8-dimethyl-1-oxaspiro[4.5]decane) was observed by GC and GC-MS (30 min). The mixture was heated to reflux and water was collected (Dean Stark apparatus). After 15 min a full conversion of 3-((8,8-dimethyl-1-oxaspiro[4.5]decan-2-yl)oxy)-2,2-dimethylpropan-1-ol and 2,2′-((2,2-dimethylpropane-1,3-diyl)bis(oxy))bis(8,8-dimethyl-1-oxaspiro[4.5]decane) was observed by GC and the mixture was stirred further 15 min. After cooling down to room temperature 2105 mg decane were added for the yield determination with internal standard. 2.196 g (8.70 mmol, 84.4% yield over 2 steps) 2-(2-(4,4-dimethylcyclohex-1-en-1-yl)ethyl)-5,5-dimethyl-1,3-dioxane and 155 mg (0.614 mmol, 6.0% yield over 2 steps) 2-(2-(4,4-dimethylcyclohexylidene)ethyl)-5,5-dimethyl-1,3-dioxane were obtained.

### Example 23

### Preparation of 2-(2-(4,4-dimethylcyclohex-1-en-1-yl)ethyl)-5,5-dimethyl-1,3-dioxane from 2-methoxy-8,8-dimethyl-1-oxaspiro[4.5]decane-acid screening

### General Procedure for Testing Several Acids:

In a 50 mL reactor (equipped with a Dean-Stark apparatus and a small column, 2 elements sulzer EX) 3.0 g of the crude of Example 20 (from 10.308 mmol 4,4-dimethyl-1-vinylcyclohexan-1-ol) were mixed after MeOH evaporation with 20 mL xylene, 3.22 g (30.92 mmol) 2,2-dimethyl-1,3-propanediol and the catalyst. After stirring at 50° C. the formation of 3-((8,8-dimethyl-1-oxaspiro[4.5]decan-2-yl)oxy)-2,2-dimethylpropan-1-ol and 2,2′-((2,2-dimethylpropane-1,3-diyl)bis(oxy))bis(8,8-dimethyl-1-oxaspiro[4.5]decane) was observed by GC and GC-MS (30 min). The mixture was heated to reflux and water was collected (Dean Stark apparatus). After the full conversion of 3-((8,8-dimethyl-1-oxaspiro[4.5]decan-2-yl)oxy)-2,2-dimethylpropan-1-ol and 2,2′-((2,2-dimethylpropane-1,3-diyl)bis(oxy))bis(8,8-dimethyl-1-oxaspiro[4.5]decane) was observed by GC the mixture was cooled down to room temperature and 2105 mg decane were added for the yield determination with internal standard.

Acid: 150 mg Clay F24X (1 h at 138° C.):

A yield of 85% of 2-(2-(4,4-dimethylcyclohex-1-en-1-yl)ethyl)-5,5-dimethyl-1,3-dioxane/2-(2-(4,4-dimethylcyclohexylidene)ethyl)-5,5-dimethyl-1,3-dioxane (ratio 93.2/6.8) was estimated.

Acid: 150 mg Clay F21X (1 h at 138° C.):

A yield of 85% of 2-(2-(4,4-dimethylcyclohex-1-en-1-yl)ethyl)-5,5-dimethyl-1,3-dioxane/2-(2-(4,4-dimethylcyclohexylidene)ethyl)-5,5-dimethyl-1,3-dioxane (ratio 93/7) was estimated.

Acid: 150 mg Zeolite CBV780 (30 Min at 138° C.)

A yield of 88.7% of 2-(2-(4,4-dimethylcyclohex-1-en-1-yl)ethyl)-5,5-dimethyl-1,3-dioxane/2-(2-(4,4-dimethylcyclohexylidene)ethyl)-5,5-dimethyl-1,3-dioxane (ratio 95.7/4.3) was obtained.

### Example 24

### Preparation of 2-(2-(4,4-dimethylcyclohex-1-en-1-yl)ethyl)-1,3-dioxepane from 2-methoxy-8,8-dimethyl-1-oxaspiro[4.5]decane Using 1,4-butandiol

In a 50 mL reactor (equipped with a Dean-Stark apparatus and a small column, 2 elements sulzer EX) 3.0 g of the crude of Example 20 (from 10.308 mmol 4,4-dimethyl-1-vinylcyclohexan-1-ol) were mixed after MeOH evaporation with 20 mL toluene, 4.64 g (51.54 mmol) 1,4-butandiol and 25.3 mg (0.258 mmol, 2.5 mol %) (98%) H₂SO₄. After stirring at 50° C. the full formation of 4-((8,8-dimethyl-1-oxaspiro[4.5]decan-2-yl)oxy)butan-1-ol and 1,4-bis((8,8-dimethyl-1-oxaspiro[4.5]decan-2-yl)oxy)butane from 2-methoxy-8,8-dimethyl-1-oxaspiro[4.5]decane was observed by GC and GC-MS (15 min). The mixture was heated to reflux during 3 hours and water was collected (Dean Stark apparatus). After cooling down to room temperature the mixture was diluted with 20 mL of MTBE and washed with 10 mL of water. The organic phase was then washed with 10 mL of a saturated aqueous NaHCO₃ solution, once with 10 mL of water, and with 10 mL of a saturated aqueous NaCl solution. After drying over sodium sulfate the solvent was evaporated under reduced pressure (50° C., 15 mbar). The crude (2.06 g) was purified by column chromatography (80 g cartridge, from cyclohexane/MTBE 98/2 to MTBE) and 2-(2-(4,4-dimethylcyclohex-1-en-1-yl)ethyl)-1,3-dioxepane (373 mg, 1.57 mmol, 15% yield), 2-(2-(4,4-dimethylcyclohexylidene)ethyl)-1,3-dioxepane (12 mg, 0.0516 mmol, 0.5% yield) were isolated. The starting material (4-((8,8-dimethyl-1-oxaspiro[4.5]decan-2-yl)oxy)butan-1-ol and 1,4-bis((8,8-dimethyl-1-oxaspiro[4.5]decan-2-yl)oxy)butane) could be isolated and reused in the same reaction.

### 2-(2-(4,4-dimethylcyclohex-1-en-1-yl)ethyl)-1,3-dioxepane

¹H-NMR (500.15 MHz): 0.88 (s, 6H), 1.35 (t, 2H, J=6.5 Hz), 1.64-1.78 (m, 8H), 1.90-1.96 (m, 2H), 1.99-2.04 (m, 2H), 3.59-3.64 (m, 2H), 3.85-3.91 (m, 2H), 4.64 (t, 1H, J=5.7 Hz), 5.31-5.35 (m, 1H).

¹³C NMR (125 MHz, CDCl₃): 326.2, 28.2, 28.5, 29.3, 32.5, 32.6, 35.7, 39.3, 65.7, 102.6, 120.0, 135.6.

### 2-(2-(4,4-dimethylcyclohexylidene)ethyl)-1,3-dioxepane

¹³C NMR (125 MHz, CDCl₃) characteristic signals: 115.3.

### 4-((8,8-dimethyl-1-oxaspiro[4.5]decan-2-yl)oxy)butan-1-ol

¹H-NMR (500.15 MHz, CDCl₃): 30.90 (s, 3H), 0.94 (s, 3H), 1.17-1.26 (m, 2H), 1.37-1.51 (m, 4H), 1.54-1.61 (m, 3H), 1.61-1.69 (m, 2H), 1.70-1.77 (m, 2H), 1.77-1.85 (m, 1H), 1.89-2.04 (m, 2H), 3.36-3.40 (m, 1H), 3.62-3.66 (m, 2H), 3.71-3.76 (m, 1H), 5.05-5.07 (m, 1H).

¹³C NMR (125 MHz, CDCl₃): 326.7, 28.1, 28.2, 29.4, 30.2, 32.4, 33.7, 33.7, 35.4, 36.6, 36.7, 62.7, 66.8, 84.5, 103.5.

### 1,4-bis((8,8-dimethyl-1-oxaspiro[4.5]decan-2-yl)oxy)butane

¹H-NMR (500.15 MHz, CDCl₃): δ 0.89 (s, 6H), 0.94 (s, 6H), 1.18-1.26 (m, 4H), 1.38-1.50 (m, 8H), 1.52-1.64 (m, 6H), 1.67-1.77 (m, 4H), 1.77-1.85 (m, 2H), 1.88-2.02 (m, 4H), 3.30-3.37 (m, 2H), 3.64-3.72 (m, 2H), 5.03-5.06 (m, 2H).

¹³C NMR (125 MHz, CDCl₃): δ 26.6, 26.7, 28.2, 28.2, 29.4, 32.5, 33.8, 35.5, 36.7, 36.8, 66.6, 66.7, 84.1, 103.5.

The product obtained was transformed according the protocol of Example 3 to 3-(4,4-dimethylcyclohex-1-en-1-yl)propanal (80° C., 4-6 h). To increase the conversion the aqueous phase was separated and a fresh water/AcOH mixture was added and the mixture was further heated at 80° C. The procedure could be repeated several times.

### Example 25

### Preparation of 3-(2-(4,4-dimethylcyclohex-1-en-1-yl)ethyl)-1,5-dihydrobenzo[e][1,3]dioxepine from 2-methoxy-8,8-dimethyl-1-oxaspiro[4.5]decane Using 1,2-Benzenedimethanol

In a 50 mL reactor (equipped with a Dean-Stark apparatus and a small column, 2 elements sulzer EX) 3.0 g of the crude of Example 20 (from 10.308 mmol 4,4-dimethyl-1-vinylcyclohexan-1-ol) were mixed after MeOH evaporation with 20 mL toluene, 4.4 g (30.9 mmol) 1,2-Benzenedimethanol and 25.3 mg (0.258 mmol, 2.5 mol %) (98%) H₂SO₄. After stirring at 50° C. the full formation of (2-(((8,8-dimethyl-1-oxaspiro[4.5]decan-2-yl)oxy)methyl)phenyl)methanol and 1,2-bis(((8,8-dimethyl-1-oxaspiro[4.5]decan-2-yl)oxy)methyl)benzene was observed by GC and GC-MS (15 min). The mixture was heated to reflux and water was collected (Dean Stark apparatus). After 15 min the formation of 3-(2-(4,4-dimethylcyclohex-1-en-1-yl)ethyl)-1,5-dihydrobenzo[e][1,3]dioxepine was observed (GC 60%). Some small amounts of 3-(2-(4,4-dimethylcyclohexylidene)ethyl)-1,5-dihydrobenzo[e][1,3]dioxepine were detected by GC/NMR.

A sample was isolated after workup (NaHCO₃, water) and column chromatography:

### 3-(2-(4,4-dimethylcyclohex-1-en-1-yl)ethyl)-1,5-dihydrobenzo[e][1,3]dioxepine

¹H-NMR (500.15 MHz): 0.89 (s, 6H), 1.36 (t, 2H, J=6.5 Hz), 1.75-1.84 (m, 4H), 1.93-1.98 (m, 2H), 2.06-2.11 (m, 2H), 4.86 (s, 4H), 4.87 (t, 1H, J=5.6 Hz), 5.34-5.38 (m, 1H), 7.15-7.23 (m, 4H).

¹³C NMR (125 MHz, CDCl₃): δ 26.2, 28.2, 28.5, 32.4, 32.7, 35.7, 39.3, 71.6, 108.3, 120.2, 127.4, 127.4, 135.4, 139.3.

### 3-(2-(4,4-dimethylcyclohexylidene)ethyl)-1,5-dihydrobenzo[e][1,3]dioxepine

¹³C NMR (125 MHz, CDCl₃) characteristic signal: 114.9.

The product obtained was transformed according the protocol of Example 3 to 3-(4,4-dimethylcyclohex-1-en-1-yl)propanal (80° C., 4-6 h). To increase the conversion the aqueous phase was separated and a fresh water/AcOH mixture was added and the mixture was further heated at 80° C. The procedure could be repeated several times.

### Example 26

### Preparation of 2-(2-(4,4-dimethylcyclohex-1-en-1-yl)ethyl)-4H-benzo[d][1,3]dioxine from 2-methoxy-8,8-dimethyl-1-oxaspiro[4.5]decane Using 2-Hydroxybenzyl alcohol

In a 50 mL reactor (equipped with a Dean-Stark apparatus and a small column, 2 elements sulzer EX) 3.0 g of the crude of Example 20 (from 10.308 mmol 4,4-dimethyl-1-vinylcyclohexan-1-ol) were mixed after MeOH evaporation with 20 mL toluene, 6.4 g (51.5 mmol) 2-Hydroxybenzyl alcohol and 351 mg (2.58 mmol, 25 mol %) KHSO₄. After stirring at 50° C. the full formation of 2-(((8,8-dimethyl-1-oxaspiro[4.5]decan-2-yl)oxy)methyl)phenol, 2-((2-((8,8-dimethyl-1-oxaspiro[4.5]decan-2-yl)oxy)benzyl)oxy)-8,8-dimethyl-1-oxaspiro[4.5]decane was observed by GC and GC-MS (15 min). The mixture was heated to reflux and water was collected (Dean Stark apparatus). After 15 min the formation of 2-(2-(4,4-dimethylcyclohexylidene)ethyl)-4H-benzo[d][1,3]dioxine (GC 42%).

A sample was isolated after workup (NaHCO₃, water) and column chromatography:

### 2-(2-(4,4-dimethylcyclohex-1-en-1-yl)ethyl)-4H-benzo[d][1,3]dioxine

¹H-NMR (500.15 MHz): 0.89 (s, 6H), 1.36 (t, 2H, J=6.4 Hz), 1.73-1.79 (m, 2H), 1.89-2.00 (m, 4H), 2.17 (t, 2H, 8.0 Hz), 4.83 (d, 1H, J=14.5 Hz) 4.97 (d, 1H, J=14.7 Hz), 5.00 (t, 1H, J=5.2 Hz), 5.36-5.40 (m, 1H), 6.83-7.17 (m, 4H).

¹³C NMR (125 MHz, CDCl₃): 326.2, 28.2, 28.2, 28.5, 31.4, 32.6, 35.7, 39.3, 66.5, 99.7, 116.7, 120.4, 120.9, 121.0, 124.9, 127.9, 135.1, 153.1.

The product obtained was transformed according the protocol of Example 3 to 3-(4,4-dimethylcyclohex-1-en-1-yl)propanal (80° C., 4-6 h). To increase the conversion the aqueous phase was separated and a fresh water/AcOH mixture was added and the mixture was further heated at 80° C. The procedure could be repeated several times.

### Example 27

### Preparation of 2-(2-(4,4-dimethylcyclohex-1-en-1-yl)ethyl)-4,7-dihydro-1,3-dioxepine from 2-methoxy-8,8-dimethyl-1-oxaspiro[4.5]decane Using (Z)-but-2-ene-1,4-diol

In a 50 mL reactor (equipped with a Dean-Stark apparatus and a small column, 2 elements sulzer EX) 3.0 g of the crude of Example 20 (from 10.308 mmol 4,4-dimethyl-1-vinylcyclohexan-1-ol) were mixed after MeOH evaporation with 20 mL toluene, 4.54 g (51.54 mmol) (Z)-but-2-ene-1,4-diol and 25.3 mg (0.258 mmol, 2.5 mol %) (98%) H₂SO₄. After stirring at 50° C. the full formation (Z)-4-((8,8-dimethyl-1-oxaspiro[4.5]decan-2-yl)oxy)but-2-en-1-ol and (Z)-1,4-bis((8,8-dimethyl-1-oxaspiro[4.5]decan-2-yl)oxy)but-2-ene from 2-methoxy-8,8-dimethyl-1-oxaspiro[4.5]decane was observed by GC and GC-MS (15 min). The mixture was heated to reflux and water was collected (Dean Stark apparatus).

A sample was isolated after workup (NaHCO₃, water) and column chromatography:

### 2-(2-(4,4-dimethylcyclohex-1-en-1-yl)ethyl)-4,7-dihydro-1,3-dioxepine

¹H-NMR (500.15 MHz): 0.88 (s, 6H), 1.35 (t, 2H, J=6.5 Hz), 1.74-1.79 (m, 4H), 1.92-1.97 (m, 2H), 2.00-2.06 (m, 2H), 4.12-4.19 (m, 2H), 4.36-4.43 (m, 2H), 4.74 (t, 1H, J=5.1 Hz), 5.32-5.36 (m, 1H), 5.70-5.73 (m, 2H).

¹³C NMR (125 MHz, CDCl₃): δ 26.2, 28.2, 28.5, 31.8, 32.6, 35.7, 39.3, 65.2, 104.3, 120.2, 129.8, 135.4.

### 2-(2-(4,4-dimethylcyclohexylidene)ethyl)-4,7-dihydro-1,3-dioxepine

¹³C NMR (125 MHz, CDCl₃) characteristic signals: 115.0.

The product obtained was transformed according the protocol of Example 3 to 3-(4,4-dimethylcyclohex-1-en-1-yl)propanal (80° C., 4-6 h). To increase the conversion the aqueous phase was separated and a fresh water/AcOH mixture was added and the mixture was further heated at 80° C. The procedure could be repeated several times.

### Example 28

### Preparation of 3,9-bis(2-(4,4-dimethylcyclohex-1-en-1-yl)ethyl)-2,4,8,10-tetraoxaspiro[5.5]undecane from 2-methoxy-8,8-dimethyl-1-oxaspiro[4.5]decane Using pentaerythritol

In a 50 mL reactor (equipped with a Dean-Stark apparatus and a small column, 2 elements sulzer EX) 3.0 g of the crude of Example 20 (from 10.308 mmol 4,4-dimethyl-1-vinylcyclohexan-1-ol) were mixed after MeOH evaporation with 20 mL toluene, 1.43 g (10.308 mmol) pentaerythritol and 1.43 g (10.308 mmol, 1 eq) KHSO₄. After stirring at 50° C. the formation of 2-(((8,8-dimethyl-1-oxaspiro[4.5]decan-2-yl)oxy)methyl)-2-(hydroxymethyl)propane-1,3-diol, (2-(2-(4,4-dimethylcyclohex-1-en-1-yl)ethyl)-1,3-dioxane-5,5-diyl)dimethanol and 2,2-bis(((8,8-dimethyl-1-oxaspiro[4.5]decan-2-yl)oxy)methyl)propane-1,3-diol was observed by GC and GC-MS (15 min). The mixture was heated to reflux and water was collected (Dean Stark apparatus). After 2 hour the formation of 3,9-bis(2-(4,4-dimethylcyclohex-1-en-1-yl)ethyl)-2,4,8,10-tetraoxaspiro[5.5]undecane (GC 86%) and (2-(2-(4,4-dimethylcyclohex-1-en-1-yl)ethyl)-1,3-dioxane-5,5-diyl)dimethanol (GC 7%) was observed (2% 3,9-bis(2-(4,4-dimethylcyclohexylidene)ethyl)-2,4,8,10-tetraoxaspiro[5.5]undecane by NMR analysis). A sample was isolated after workup (NaHCO₃, water) and column chromatography:

### 3,9-bis(2-(4,4-dimethylcyclohex-1-en-1-yl)ethyl)-2,4,8,10-tetraoxaspiro[5.5]undecane

¹H-NMR (500.15 MHz): 0.88 (s, 12H), 1.35 (t, 4H, J=6.5 Hz), 1.67-1.78 (m, 8H), 1.89-1.94 (m, 4H), 2.00-2.06 (m, 4H), 3.32 (d, 2H, J=11.6 Hz), 3.51 (d, 2H, J=11.6 Hz), 3.56 (dd, 2H, J=11.6 Hz, J=2.6 Hz), 4.42 (t, 2H, J=5.0 Hz), 4.55 (dd, 2H, J=11.4 Hz, J=2.3 Hz), 5.28-5.33 (m, 2H).

¹³C NMR (125 MHz, CDCl₃): 326.1, 28.2, 28.5, 31.5, 32.4, 33.0, 35.7, 39.3, 70.2, 70.6, 102.7, 120.1, 135.4.

### 3,9-bis(2-(4,4-dimethylcyclohexylidene)ethyl)-2,4,8,10-tetraoxaspiro[5.5]undecane

¹³C NMR (125 MHz, CDCl₃) characteristic signal: 3114.1, 142.3.

### 2-(2-(4,4-dimethylcyclohex-1-en-1-yl)ethyl)-1,3-dioxane-5,5-diyl)dimethanol

¹³C NMR (125 MHz, CDCl₃) characteristic signal: 364.4, 65.9.

The product obtained was transformed according the protocol of Example 3 to 3-(4,4-dimethylcyclohex-1-en-1-yl)propanal (80° C., 4-6 h). To increase the conversion the aqueous phase was separated and a fresh water/AcOH mixture was added and the mixture was further heated at 80° C. The procedure could be repeated several times.

### Example 29

### Preparation of 2-(2-(4,4-dimethylcyclohex-1-en-1-yl)ethyl)-1,3-dioxan-5-ol/(2-(2-(4,4-dimethylcyclohex-1-en-1-yl)ethyl)-1,3-dioxolan-4-yl)methanol from 2-methoxy-8,8-dimethyl-1-oxaspiro[4.5]decane Using 1,2,3-propanetriol

In a 50 mL reactor (equipped with a Dean-Stark apparatus and a small column, 2 elements sulzer EX) 3.0 g of the crude of Example 20 (from 10.308 mmol 4,4-dimethyl-1-vinylcyclohexan-1-ol) were mixed after MeOH evaporation with 20 mL xylene, 2.85 g (51.54 mmol) 1,2,3-propanetriol and 25.3 mg (0.258 mmol, 2.5 mol %) (98%) H₂SO₄. After stirring at 50° C. the formation of 3-((8,8-dimethyl-1-oxaspiro[4.5]decan-2-yl)oxy)propane-1,2-diol and 1,3-bis((8,8-dimethyl-1-oxaspiro[4.5]decan-2-yl)oxy)propan-2-ol was observed by GC and GC-MS (15 min). The mixture was heated to reflux and water was collected (Dean Stark apparatus, after 2 h 2 eq of 1,2,3-propanetriol were added). After 3 hour (further 1,2,3-propanetriol was added during the heating) the quantitative formation of 2-(2-(4,4-dimethylcyclohex-1-en-1-yl)ethyl)-1,3-dioxan-5-ol/(2-(2-(4,4-dimethylcyclohex-1-en-1-yl)ethyl)-1,3-dioxolan-4-yl)methanol was observed (GC 97%, 2.26 g crude after workup, 2.11 g (8.78 mmol) after distillation, 85% yield over 2 steps). 2% 2-(2-(4,4-dimethylcyclohexylidene)ethyl)-1,3-dioxan-5-ol/(2-(2-(4,4-dimethylcyclohexylidene)ethyl)-1,3-dioxolan-4-yl)methanol were observed by GC/NMR.

A sample was isolated after workup (NaHCO₃, water) and column chromatography:

### 2-(2-(4,4-dimethylcyclohex-1-en-1-yl)ethyl)-1,3-dioxan-5-ol/(2-(2-(4,4-dimethylcyclohex-1-en-1-yl)ethyl)-1,3-dioxolan-4-yl)methanol

¹H-NMR (500.15 MHz) mixture of 4 isomers (5/3/1/1): 0.88 (s, 6H), 1.36 (t, 2H, J=6.5 Hz), 1.68-1.85 (m, 4H), 1.89-1.97 (m, 2H), 2.01-2.10 (m, 2H), 3.33-4.24 (m, 5H), 4.38 (t, 0.1H, J=5.2 Hz), 4.54 (t, 0.1H, J=5.1 Hz), 4.91 (t, 0.5H, J=4.7 Hz), 4.91 (t, 0.3H, J=4.8 Hz), 5.30-5.36 (m, 1H).

¹³C NMR (125 MHz, CDCl₃ characteristic signals): δ 26.1, 26.2, 26.2, 28.2, 28.2, 28.2, 28.2, 28.5, 28.5, 31.5, 31.7, 31.7, 31.9, 32.1, 32.4, 32.5, 33.1 35.7, 39.3, 39.3, 39.3, 61.5, 62.7, 63.4, 64.1, 66.3, 66.5, 71.5, 71.8, 76.1, 76.2, 101.8, 102.5, 104.6, 104.9, 120.1, 120.2, 120.2, 135.3, 135.3, 135.4.

### 2-(2-(4,4-dimethylcyclohexylidene)ethyl)-1,3-dioxan-5-ol/(2-(2-(4,4-dimethylcyclohexylidene)ethyl)-1,3-dioxolan-4-yl)methanol

¹³C NMR (125 MHz, CDCl₃ characteristic signals): 113.5, 113.8, 113.9.

The product obtained was transformed according the protocol of Example 3 to 3-(4,4-dimethylcyclohex-1-en-1-yl)propanal (80° C., 4-6 h). To increase the conversion the aqueous phase was separated and a fresh water/AcOH mixture was added and the mixture was further heated at 80° C. The procedure could be repeated several times.

### Example 30

### Hydroformylation of 4,4-dimethyl-1-vinylcyclohexan-1-ol in glycerol

The autoclave was charged with 4,4-dimethyl-1-vinylcyclohexan-1-ol (7.54 g, 48.882 mmol), glycerol (13.65 g, 148.22 mmol), BiPhePhos (0.1148 mg, 1×10⁻⁴ mmol) and Rh(CO)₂acac (0.0125 mg, 4.86×10−5 mmol). The vessel was purged with H₂/CO 1:1 (4×5 bar) and pressurized at 10 bar and heated 60 h at 90° C. under vigorous stirring to reach 93.6% conversion. After cooling and depressurization, the crude mixture was diluted with Et₂O (20 ml) and washed with H₂O (20 ml), the aqueous layer was decanted and extracted with a second portion of Et₂O (20 ml). The combined organic layers were dried with anhydrous sodium sulfate, filtered and concentrated to give 11.24 g of residue. GLC analysis of the residue revealed the presence of starting material (6.3%), 3-((8,8-dimethyl-1-oxaspiro[4.5]decan-2-yl)oxy)propane-1,2-diol (44.7%), 1,3-bis((8,8-dimethyl-1-oxaspiro[4.5]decan-2-yl)oxy)propan-2-ol (33.5%) and hydrogenated starting material (6.6%).

3-((8,8-dimethyl-1-oxaspiro[4.5]decan-2-yl)oxy)propane-1,2-diol and 1,3-bis((8,8-dimethyl-1-oxaspiro[4.5]decan-2-yl)oxy)propan-2-ol were isolated by chromatography (Et₂O/pentane—1:1) and identified by spectral means.

### 3-((8,8-dimethyl-1-oxaspiro[4.5]decan-2-yl)oxy)propane-1,2-diol (Isomer Mixture)

¹H-NMR (500.15 MHz, CDCl₃): δ 0.90 (s, 3H), 0.94 (s, 3H), 1.18-2.25, 3.51-3.88 (overlapping multiplets, 20H), 5.07 (overlapping triplets, 2H).

¹³C NMR (125 MHz, CDCl₃): δ 28.22 (q, br), 29.42 (s, br), 32.38, 32.42 (t, br), 33.44, 33.46 (t), 33.8 (t, br), 35.22, 35.27 (t), 36.5, 36.6 (t, br), 36.63, 36.68 (t), 63.99, 64.23 (t), 70.1 (t), 70.74 (d), 70.99 (d), 71.68 (t), 85.2 (s), 104.93, 105.14 (d).

### 1,3-bis((8,8-dimethyl-1-oxaspiro[4.5]decan-2-yl)oxy)propan-2-ol

¹H-NMR (500.15 MHz, CDCl₃): δ 0.90 (s, 3H), 0.94 (s, 3H), 1.15-2.07, 3.45-3.9 (overlapping multiplets, 20H), 5.07 (overlapping triplets, 2H).

¹³C NMR (125 MHz, CDCl₃): δ 28.18 (q, br), 29.40 (s), 32.42, 32.45 (t, br), 33.61 (overlapping t), 33.8 (t, br), 35.33, 35.39 (t), 36.57 (t, br), 36.71, 36.73 (t), 69.01 (t), 67.74 (d), 79.9 (d), 70.3 (t), 84.7, 84.8 (s), 104.45, 104.65 (d).

### Example 31

### Hydroformylation of 4-(tert-butyl)-1-vinylcyclohexan-1-ol

The autoclave was charged with 4-tert-butyl-1-vinyl-cyclohexanol mixture (5.04 g, 27.647 mmol, 43/51 cis/trans), 1,2-ethanediol (5.98 g, 96.343 mmol), BiPhePhos (32.9 mg, 0.0418 mmol) and Rh(CO)₂acac (3.8 mg, 0.0147 mmol). The vessel was purged with H₂/CO 1:1 (4×5 bar), pressurized at 10 bar and then heated under vigorous stirring at 90° C. and constant pressure for 48 h to reach >99% conversion. A sample (1.65 g) of the crude solid reaction mixture (10.89 g) was dissolved in Et₂O (20 ml) and washed with water (10 ml). The organic phase was dried over anhydrous sodium sulfate, filtered and concentrated to give a white gluey solid (1.27 g) which was analyzed by GLC to reveal the presence of a mixture of hydrogenated starting material 4-(tert-butyl)-1-ethylcyclohexan-1-ol (4.1%), 2-(((2RS,5s,8RS)— and 2-(((2SR,5r,8RS)-8-(tert-butyl)-1-oxaspiro[4.5]decan-2-yl)oxy)ethan-1-ol (80.4%), 1,2-bis(((2SR,5r,8RS)-8-(tert-butyl)-1-oxaspiro[4.5]decan-2-yl)oxy)ethane (˜5-7%) and 8-(tert-butyl)-1,4-dioxaspiro[4.5]decane (5-7%, resulting from an impurity in the starting material). The products were isolated by chromatography (Et₂O/pentane—1:1) and identified by spectral means. The crude reaction mixture remaining after the analyses (9.24 g) was further processed in the next step.

### 2-(((2RS,5s,8RS)-8-(tert-butyl)-1-oxaspiro[4.5]decan-2-yl)oxy)ethan-1-ol

¹H-NMR (500.15 MHz, CDCl₃): δ 0.86 (s, 9H), 0.94-1.0 (m, 1H), 1.25-1.40 (m, 3H), 1.46 (td, 1H), 1.58-1.63 (m, 1H), 1.63-1.69 (m, 2H), 1.71-1.76 (m, 1H), 1.84-1.89 (m, 2H), 1.93-1.99 (m, 1H), 2.03-2.11 (m, 1H), 3.27 (dd, 1H), 3.62-3.78 (m, 4H), 5.09 (d, 1H).

¹³C NMR (150 MHz, CDCl₃): 323.4 (t), 23.9 (t), 27.6 (q), 32.3 (t), 32.4 (s), 36.2 (t), 38.2 (t), 38.9 (t), 47.4 (d), 62.7 (t), 71.1 (t), 83.5 (s), 105.3 (d).

### 2-(((2SR,5r,8RS)-8-(tert-butyl)-1-oxaspiro[4.5]decan-2-yl)oxy)ethan-1-ol

¹H-NMR (500.15 MHz, CDCl₃): δ 0.85 (s, 9H), 0.99-1.05 (m, 1H), 1.43-1.45 (m, 1H), 1.59-1.70 (m, 3H), 1.72-1.88 (m, 6H), 1.96-2.10 (m, 2H), 3.16 (dd, 1H), 3.61-3.76 (m, 4H), 5.08 (d, 1H).

¹³C NMR (150 MHz, CDCl₃): 325.2 (t), 25.7 (t), 27.6 (q), 31.8 (t), 32.2 (s), 32.7 (t), 37.4 (t), 40.1 (t), 47.1 (d), 62.5 (t), 70.8 (t), 85.9 (s), 104.4 (d). 1,2-bis(((2SR,5r,8RS)-8-(tert-butyl)-1-oxaspiro[4.5]decan-2-yl)oxy)ethane

¹H-NMR (500.15 MHz, CDCl₃): δ 0.85 (s, 18H), 0.97-1.04 (m, 4H), 1.25-2.02 (m, 22H), 3.54 (m, 2H), 3.77 (m, 2H), 5.08 (d, 2H).

¹³C NMR (150 MHz, CDCl₃): δ 25.2 (t), 25.8 (t), 27.6 (q), 31.6 (t), 32.2 (s), 32.7 (t), 37.6 (t), 40.3 (t), 47.2 (d), 65.6 (t), 85.3 (s), 103.1 (d).

### Example 32

### Preparation of 2-(2-(4-(tert-butyl)cyclohex-1-en-1-yl)ethyl)-1,3-dioxolane from 2-((8-(tert-butyl)-1-oxaspiro[4.5]decan-2-yl)oxy)ethan-1-ol and 1,2-bis((8-(tert-butyl)-1-oxaspiro[4.5]decan-2-yl)oxy)ethane

In a 100 mL reactor (equipped with a Dean-Stark apparatus and a small column, 2 elements sulzer EX) 9.24 g of the crude of Example 31 (hydroformylation reaction, 22.001 mmol 4-tert-butyl-1-vinyl-cyclohexanol) was stirred with 20 mL Xylene, 6.83 g (110.01 mmol) ethylene glycol and 54.9 mg (0.55 mmol, 2.5 mol %) (98%) H₂SO₄. The mixture was heated to 130° C.-138° C. and water was collected (Dean Stark apparatus). After 3 hours 97% conversion of 2-((8-(tert-butyl)-1-oxaspiro[4.5]decan-2-yl)oxy)ethan-1-ol and 1,2-bis((8-(tert-butyl)-1-oxaspiro[4.5]decan-2-yl)oxy)ethane was observed by GC. After cooling down to room temperature the mixture was diluted with 20 mL of MTBE and washed with 10 mL of water. The organic phase was then washed with 10 mL of a saturated aqueous NaHCO₃ solution, once with 10 mL of water, and with 10 mL of a saturated aqueous NaCl solution. After drying over sodium sulfate the solvent was evaporated under reduced pressure (50° C., 15 mbar). The crude (5.37 g) was distilled (Kugelrohr) for the purification. 3.445 g (14.452 mmol) 2-(2-(4-(tert-butyl)cyclohex-1-en-1-yl)ethyl)-1,3-dioxolane were obtained (66% yield over 2 steps) and 244 mg (1.023 mmol, 5% yield over 2 steps) 2-(2-(4-(tert-butyl)cyclohexylidene)ethyl)-1,3-dioxolane).

### 2-(2-(4-(tert-butyl)cyclohex-1-en-1-yl)ethyl)-1,3-dioxolane

¹H-NMR (500.15 MHz): 0.89 (t, 3H, J=7.0 Hz), 1.14-1.34 (m, 7H), 1.39-1.40 (m, 1H), 1.55-1.65 (m, 1H), 1.70-1.79 (m, 3H), 1.89-2.12 (m, 5H), 3.82-3.89 (m, 2H), 3.92-4.01 (m, 2H), 4.85 (t, 1H, J=4.8 Hz), 5.38-5.42 (m, 1H). 13C NMR (125 MHz, CDCl₃): δ 14.2, 23.0, 28.5, 29.3, 29.4, 31.9, 32.1, 32.2, 33.5, 36.2, 64.9, 104.5, 120.6, 136.8.

### 2-(2-(4-(tert-butyl)cyclohexylidene)ethyl)-1,3-dioxolane

¹³C NMR (125 MHz, CDCl₃) characteristic signal: 113.7.

### Example 33

### Preparation of 3-(4-(tert-butyl)cyclohex-1-en-1-yl)propanal

The compound was prepared according to procedure reported in Example 3 using, as a starting material, the compound prepared in Example 32.

The ¹H and ¹³C-NMR analysis results in CDCl₃ were in accordance with data from literature (see B. Winter EP 1054053 A2).

### Example 34

### Hydroformylation of (1RS,3SR)— and (1SR,3SR)-3-isopropyl-1-vinylcyclohexan-1-ol

The autoclave was charged with freshly distilled (1RS,3SR)— and (1SR,3SR)-3-isopropyl-1-vinylcyclohexan-1-ol (5.01 g, 29.772 mmol, 39.6/48.3 diastereomeric mixture, contaminated with ≈10% cis/trans-4-isopropyl-1-vinylcyclohexan-1-ol), 1,2-ethanediol (6.46 g, 104.08 mmol), BiPhePhos (35.4 mg, 0.045 mmol) and Rh(CO)₂acac (3.9 mg, 0.0151 mmol). The vessel was purged with H₂/CO 1:1 (4×5 bar), pressurized at 10 bar and then heated under vigorous stirring at 90° C. and constant pressure for 48 h to reach >99% conversion. The crude mixture (˜12 g) was diluted with Et₂O (50 ml) and washed with water (2×20 ml). The organic phase was dried over anhydrous sodium sulfate, filtered and concentrated to give yellow oil (5.9 g) which was analyzed by GLC to reveal the presence of a mixture of 2-((7-isopropyl-1-oxaspiro[4.5]decan-2-yl)oxy)ethan-1-ol (81.5%) and only traces of 1,2-bis((7-isopropyl-1-oxaspiro[4.5]decan-2-yl)oxy)ethane. Cis/trans-4-isopropyl-1-vinyl-cyclohexan-1-ol was also converted into 2-((8-isopropyl-1-oxaspiro[4.5]decan-2-yl)oxy)ethan-1-ol (≈12%). The crude reaction mixture (5.9 g) was further processed in the next step.

### 2-((7-isopropyl-1-oxaspiro[4.5]decan-2-yl)oxy)ethan-1-ol (4 Diastereomers)

¹H-NMR (500.15 MHz, CDCl₃): δ 0.82-0.88 (m, 6H), 0.95-2.14 (m, 15H), 3.6-3.8 (m, 4H), 5.06-5.2 (m, 1H).

¹³C NMR (150 MHz, CDCl₃): δ 19.3, 19.8, 19.6, 19.67, 19.73, 19.75, 19.78, 19.82 (q), 22.5, 23.0, 23.9, 24.4 (t), 28.4, 28.47, 28.5, 29.0 (t), 32.16, 32.2, 32.4 (t), 32.59, 32.63, 32.66, 32.72 (d), 32.80, 32.81 (t), 36.9, 37.1, 37.8, 38.4 (t), 39.5 (d), 39.9 (t), 40.3 (d), 40.8 (t), 41.4 (t), 41.8 (t), 42.3, 42.7 (d), 43.6 (t), 62.5, 62.55, 62.59, 62.62 (t), 70.8, 70.81, 70.98, 71.03 (t), 84.56, 84.65, 86.45, 86.57 (s), 104.30, 104.38, 105.31, 105.34 (d).

### Example 35

### Preparation of 2-(2-(5-isopropylcyclohex-1-en-1-yl)ethyl)-1,3-dioxolane and 2-(2-(3-isopropylcyclohex-1-en-1-yl)ethyl)-1,3-dioxolane from 2-((7-isopropyl-1-oxaspiro[4.5]decan-2-yl)oxy)ethan-1-ol and 1,2-bis((7-isopropyl-1-oxaspiro[4.5]decan-2-yl)oxy)ethane

In a 100 mL reactor (equipped with a Dean-Stark apparatus and a small column, 2 elements sulzer EX) 5.01 g of the crude obtained of Example 34 (hydroformylation reaction, 26.795 mmol 3-isopropyl-1-vinylcyclohexan-1-ol, and 2.98 mmol cis/trans-4-isopropyl-1-vinylcyclohexan-1-ol) was stirred with 50 mL xylene, 8.32 g (133.97 mmol) ethylene glycol and 66.7 mg (0.67 mmol, 2.5 mol %) (98%) H₂SO₄. The mixture was heated to 130° C.-138° C. and water was collected (Dean Stark apparatus). After 2 hours a full conversion of 2-((7-isopropyl-1-oxaspiro[4.5]decan-2-yl)oxy)ethan-1-ol and 1,2-bis((7-isopropyl-1-oxaspiro[4.5]decan-2-yl)oxy)ethane was observed by GC. After cooling down to room temperature the mixture was diluted with 20 mL of MTBE and washed with 10 mL of water. The organic phase was then washed with 10 mL of a saturated aqueous NaHCO₃ solution, once with 10 mL of water, and with 10 mL of a saturated aqueous NaCl solution. After drying over sodium sulfate the solvent was evaporated under reduced pressure (50° C., 15 mbar). The crude (5.23 g) was distilled (Kugelrohr) for the purification. 3.57 g (15.913 mmol) 2-(2-(5-isopropylcyclohex-1-en-1-yl)ethyl)-1,3-dioxolane and 2-(2-(3-isopropylcyclohex-1-en-1-yl)ethyl)-1,3-dioxolane were obtained (˜1/1 mixture, 59% yield over 2 steps, and 307 mg (1.36 mmol, 5% yield over 2 steps) 2-(2-(3-isopropylcyclohexylidene)ethyl)-1,3-dioxolane). 368 mg (1.64 mmol) 2-(2-(4-isopropylcyclohex-1-en-1-yl)ethyl)-1,3-dioxolane were also isolated.

### 2-(2-(5-isopropylcyclohex-1-en-1-yl)ethyl)-1,3-dioxolane/2-(2-(3-isopropylcyclohex-1-en-1-yl)ethyl)-1,3-dioxolane 1/1 Mixture

¹H-NMR (500.15 MHz): 0.82-0.92 (m, 6H), 1.06-1.35 (m, 2H), 1.39-1.59 (m, 1H), 1.63-2.10 (m, 9H), 3.81-3.90 (m, 2H), 3.91-4.00 (m, 2H), 4.85 (t, 0.5H, J=4.8 Hz) 4.86 (t, 0.5H, J=4.8 Hz), 5.34 (m, 0.5H), 5.41 (m, 0.5H).

### 2-(2-(5-isopropylcyclohex-1-en-1-yl)ethyl)-1,3-dioxolane

¹³C NMR (125 MHz, CDCl₃): δ 19.6, 19.9, 25.9, 26.0, 32.1, 32.2, 32.2, 32.4, 40.5, 64.9, 104.5, 120.8, 136.8.

### 2-(2-(3-isopropylcyclohex-1-en-1-yl)ethyl)-1,3-dioxolane

¹³C NMR (125 MHz, CDCl₃): δ 19.3, 19.6, 22.6, 25.4, 28.7, 32.2, 32.3, 32.4, 41.8, 64.9, 104.4, 124.9, 137.2.

### 2-(2-(4-isopropylcyclohex-1-en-1-yl)ethyl)-1,3-dioxolane (Characterized from the Mixture)

¹³C NMR (100 MHz, CDCl₃): δ 19.7, 20.0, 26.4, 28.9, 29.1, 31.8, 32.2, 32.3, 40.2, 64.8, 104.6, 120.9, 136.7.

### Example 36

### Preparation of 3-(5-isopropylcyclohex-1-en-1-yl)propanal/3-(3-isopropylcyclohex-1-en-1-yl)propanal/and 10% (3-(4-isopropylcyclohex-1-en-1-yl)propanal)

The compound (˜1/1 mixture) was prepared according to procedure reported in Example 3 using, as a starting material, the compound prepared in Example 36. The ¹H and ¹³C-NMR analysis results in CDCl₃ were in accordance with data from literature (see R. Moretti, A. Birkbeck WO 2017046071 A1).

### Example 37

### Hydroformylation of 4-isopropyl-1-vinylcyclohexan-1-ol

The autoclave was charged with 4-isopropyl-1-vinyl-cyclohexan-1-ol (5.08 g, 30.188 mmol, 35/59 cis/trans), 1,2-ethanediol (6.56 g, 105.69 mmol), BiPhePhos (36.3 mg, 0.0461 mmol) and Rh(CO)₂acac (4.3 mg, 0.0167 mmol). The vessel was purged with H₂/CO 1:1 (4×5 bar), pressurized at 10 bar and then heated under vigorous stirring at 90° C. and constant pressure for 48 h to reach >99% conversion. The crude yellow oil (11.2 g) was analyzed by GLC to reveal the presence of a mixture of hydrogenated starting material cis/trans 4-isopropyl-1-vinyl-cyclohexan-1-ol (≈2%/4%), 2-((−8-isopropyl-1-oxaspiro[4.5]decan-2-yl)oxy)ethan-1-ols (20%/36%) and 1,2-bis((-8-isopropyl-1-oxaspiro[4.5]decan-2-yl)oxy)ethanes (4.2%/7.3%/5%/3.9%/3.9%). The products were isolated by chromatography (Et₂O/pentane—1:1) and identified by spectral means. Among the stereoisomeric 1,2-bis((-8-isopropyl-1-oxaspiro[4.5]decan-2-yl)oxy)ethanes, only the stereochemical structure of 1,2-bis(((2SR,5r,8RS)-8-isopropyl-1-oxaspiro[4.5]decan-2-yl)oxy)ethane could be assigned exactly. The crude reaction mixture remaining after the analyses (10.2 g) was further processed in the next step.

### 2-(((2RS,5s,8RS)-8-isopropyl-1-oxaspiro[4.5]decan-2-yl)oxy)ethan-1-ol

¹H-NMR (500.15 MHz, CDCl₃): δ 0.87 (s, 3H), 0.88 (s, 3H), 0.97-1.06 (m, 1H), 1.24-1.74 (m, 10H), 1.81-1.88 (m, 2H), 1.93-1.99 (m, 1H), 2.03-2.11 (m, 1H), 3.62-3.77 (m, 4H), 5.09 (d, 1H).

¹³C NMR (150 MHz, CDCl₃): δ 19.9 (q), 20.0 (q), 25.8 (t), 26.4 (t), 32.3 (t), 32.5 (d), 36.1 (t), 37.7 (t), 38.5 (t), 43.2 (d), 62.7 (t), 71.1 (t), 83.8 (s), 105.2 (d).

### 2-(((2SR,5r,8RS)-8-isopropyl-1-oxaspiro[4.5]decan-2-yl)oxy)ethan-1-ol

¹H-NMR (500.15 MHz, CDCl₃): δ 0.86 (s, 3H), 0.87 (s, 3H), 1.02-1.06 (m, 1H), 1.38-1.75 (m, 10H), 1.78-1.85 (m, 2H), 1.95-2.02 (m, 1H), 2.02-2.09 (m, 1H), 3.62-3.75 (m, 4H), 5.08 (d, 1H).

¹³C NMR (150 MHz, CDCl₃): δ 20.0 (q), 27.6 (t), 27.9 (t), 31.8 (t), 32.3 (d), 32.7 (t), 37.0 (t), 39.7 (t), 43.0 (d), 62.5 (t), 70.8 (t), 86.0 (s), 104.4 (d).

### 1,2-bis(((2SR,5r,8RS)-8-isopropyl-1-oxaspiro[4.5]decan-2-yl)oxy)ethane

¹H-NMR (500.15 MHz, CDCl₃): δ 0.85 (s, 6H), 0.86 (s, 6H), 0.95-2.03 (m, 28H), 3.50-3.57 (m, 2H), 3.74-3.84 (m, 2H), 5.08 (d, 2H).

¹³C NMR (150 MHz, CDCl₃): δ 20.0 (q), 20.0 (q), 27.5 (t), 28.1 (t), 31.7 (t), 32.3 (d), 32.7 (t), 37.2 (t), 40.0 (t), 43.1 (d), 65.5 (t), 85.4 (s), 103.1 (d).

### Example 38

### Preparation of 2-(2-(4-isopropylcyclohex-1-en-1-yl)ethyl)-1,3-dioxolane from 2-((8-isopropyl-1-oxaspiro[4.5]decan-2-yl)oxy)ethan-1-ol and 1,2-bis((8-isopropyl-1-oxaspiro[4.5]decan-2-yl)oxy)ethane

In a 100 mL reactor (equipped with a Dean-Stark apparatus and a small column, 2 elements sulzer EX) 10.2 g of the crude of Example 37 (hydroformylation reaction, 26.124 mmol 4-isopropyl-1-vinyl-cyclohexan-1-ol) was stirred with 20 mL xylene, 8.11 g (130.6 mmol) ethylene glycol and 64.1 mg (0.65 mmol, 2.5 mol %) (98%) H₂SO₄. The mixture was heated to 130° C.-138° C. and water was collected (Dean Stark apparatus). After 4 hours 97% conversion of 2-((8-isopropyl-1-oxaspiro[4.5]decan-2-yl)oxy)ethan-1-ol and 1,2-bis((8-isopropyl-1-oxaspiro[4.5]decan-2-yl)oxy)ethane was observed by GC. After cooling down to room temperature the mixture was diluted with 20 mL of MTBE and washed with 10 mL of water. The organic phase was then washed with 10 mL of a saturated aqueous NaHCO₃ solution, once with 10 mL of water, and with 10 mL of a saturated aqueous NaCl solution. After drying over sodium sulfate the solvent was evaporated under reduced pressure (50° C., 15 mbar). The crude (5.02 g) was distilled (Kugelrohr) for the purification. 3.37 g (15.022 mmol) 2-(2-(4-isopropylcyclohex-1-en-1-yl)ethyl)-1,3-dioxolane were obtained (58% yield over 2 steps) and 279 mg (1.24 mmol, 5% yield over 2 steps) 2-(2-(4-isopropylcyclohexylidene)ethyl)-1,3-dioxolane).

### 2-(2-(4-isopropylcyclohex-1-en-1-yl)ethyl)-1,3-dioxolane

¹H-NMR (500.15 MHz): 0.87 (d, 3H, J=5.9 Hz), 0.89 (d, 3H, J=5.9 Hz), 1.10-1.31 (m, 2H), 1.38-1.52 (m, 1H), 1.65-1.81 (m, 4H), 1.88-2.10 (m, 5H), 3.82-3.90 (m, 2H), 3.92-4.01 (m, 2H), 4.86 (t, 1H, J=4.9 Hz), 5.39-5.45 (m, 1H).

¹³C NMR (125 MHz, CDCl₃): δ 19.7, 20.0, 26.4, 28.9, 29.1, 31.8, 32.2, 32.3, 40.2, 64.8, 104.6, 120.9, 136.7.

### 2-(2-(4-isopropylcyclohexylidene)ethyl)-1,3-dioxolane

¹³C NMR (125 MHz, CDCl₃) characteristic signal: 113.7.

### Example 39

### Preparation of 3-(4-isopropylcyclohex-1-en-1-yl)propanal

The compound was prepared according to procedure reported in Example 3 using, as a starting material, the compound prepared in Example 38. ¹H and ¹³C-NMR analysis results in CDCl₃ were in accordance with data from literature (E. Singer, B, Holscher, US2013/90390, 2013, A1).

### Example 40

### Hydroformylation of 3-isopropyl-1-vinyl-cyclopentanol

The autoclave was charged with (1RS,3SR)—/(1RS,3RS)-3-isopropyl-1-vinyl-cyclopentanol (5.04 g, 32.48 mmol, (1RS,3SR)—/(1RS,3RS)=54.6/42.1), 1,2-ethanediol (7.17 g, 115.51 mmol), Rh(CO)₂acac (4.2 mg, 0.0163 mmol) and BiPhePhos (34.4 mg, 0.0488 mmol). The vessel was purged with H₂/CO, 1:1 (4×5 bar), pressurized at 10 bar and then heated under vigorous stirring at 90° C. for 48 h at constant pressure to reach >99% conversion. A sample (250 mg) of the crude mixture (11.39 g) was diluted with Et₂O (2 ml) and washed with water (2×1 ml). The organic phase was dried over anhydrous sodium sulfate, filtered and concentrated to give a pale-yellow oil (128.1 mg).

Spectral analysis of the pale yellow oil revealed the presence of 2-((7-isopropyl-1-oxaspiro[4.4]nonan-2-yl)oxy)ethan-1-ol (62.2%) and 1,2-bis((7-isopropyl-1-oxaspiro[4.4]nonan-2-yl)oxy)ethane (25%). The crude reaction mixture remaining after the analyses (11.14 g) was further processed in the next step. 2-((7-isopropyl-1-oxaspiro[4.4]nonan-2-yl)oxy)ethan-1-ol (4 isomers) ¹H-NMR (500.15 MHz, CDCl₃): δ 0.85-0.90 (m, 6H), 1.13-2.17 (m, 13H), 3.6-3.8 (m, 4H), 5.05-5.13 (m, 1H).

¹³C NMR (150 MHz, CDCl₃): δ 21.15, 21.16, 21.019, 21.22, 21.35, 21.39, 21.44, 21.49 (q), 28.54, 28.97, 29.07, 29.15 (t), 33.02, 33.15, 33.20, 33.23 (t), 33.65, 33.74, 33.80, 33.84 (d), 34.85, 35.11, 35.79, 35.96 (t), 38.58, 38.92, 39.20, 39.59 (t), 43.97, 44.06, 44.37, 44.58 (t), 45.71, 45.92, 45.97, 46.15 (d), 62.57, 62.62, 62.64, 62.65 (t), 71.13, 71.17, 71.19, 71.31 (t), 92.13, 92.23, 92.94, 93.05 (s), 104.92, 104.99, 105.11, 105.23 (d).

### 1,2-bis((7-isopropyl-1-oxaspiro[4.4]nonan-2-yl)oxy)ethane (Complex Isomer Mixture)

¹H-NMR (500.15 MHz): δ 0.83-0.95 (m, 12H), 1.13-2.17 (m, 24H), 3.49-3.86 (m, 4H), 5.06-5.14 (m, 2H).

¹³C NMR (150 MHz, CDCl₃): δ 21.06-21.58 (series of q), 28.38-29.5 (series of t), 32.95-33.38 (series of t), 33.58-33.95 (series of d), 34.64-35.15 (series of t), 35.58-36.01 (series of t), 38.51-40.05 (series of t), 44.12-45.11 (series of t), 45.58-46.45 (series of d), 65.71-66.30 (series of t), 91.48-92.48 (series of s), 103.44-104.10 (series of d).

### Example 41

### Preparation of 2-(2-(4-isopropylcyclopent-1-en-1-yl)ethyl)-1,3-dioxolane and 2-(2-(3-isopropylcyclopent-1-en-1-yl)ethyl)-1,3-dioxolane from 2-((7-isopropyl-1-oxaspiro[4.4]nonan-2-yl)oxy)ethan-1-ol and 1,2-bis((7-isopropyl-1-oxaspiro[4.4]nonan-2-yl)oxy)ethane

In a 100 mL reactor (equipped with a Dean-Stark apparatus and a small column, 2 elements sulzer EX) 3.0 g of the crude of Example 40 (hydroformylation reaction, 8.32 mmol 3-isopropyl-1-vinyl-cyclopentanol) was stirred with 20 mL Xylene, 1.03 g (16.64 mmol) ethylene glycol and 20.4 mg (0.208, 2.5 mol %) (98%) H₂SO₄. The mixture was heated to 130° C.-138° C. and water was collected (Dean Stark apparatus). After 3 hours a full conversion of 2-((7-isopropyl-1-oxaspiro[4.4]nonan-2-yl)oxy)ethan-1-ol and 1,2-bis((7-isopropyl-1-oxaspiro[4.4]nonan-2-yl)oxy)ethane was observed by GC. After cooling down to room temperature 1.45 g decane were added for the yield determination with internal standard. 727 mg (3.456 mmol) 2-(2-(4-isopropylcyclopent-1-en-1-yl)ethyl)-1,3-dioxolane and 545 mg (2.59 mmol) 2-(2-(3-isopropylcyclopent-1-en-1-yl)ethyl)-1,3-dioxolane were obtained (72% yield over 2 steps) and 116 mg (0.55 mmol) 2-(2-(3-isopropylcyclopentylidene)ethyl)-1,3-dioxolane 7% yield over 2 steps).

### 2-(2-(4-isopropylcyclopent-1-en-1-yl)ethyl)-1,3-dioxolane/2-(2-(3-isopropylcyclopent-1-en-1-yl)ethyl)-1,3-dioxolane ˜1/1 Mixture

¹H-NMR (500.15 MHz): 0.83, 0.86, 0.87, 0.87 (d, 6H, J=6.7 Hz), 1.42-1.54 (m, 1.5H) 1.77-1.84 (m, 2H), 1.92-2.02 (m, 2H), 2.12-2.45 (m, 4.5H), 3.82-3.89 (m, 2H), 3.93-4.01 (m, 2H), 4.87 (t, J=4.8 Hz, 0.5H), 4.88 (t, J=4.8 Hz, 0.5H), 5.29-5.32 (m, 0.5H), 5.33-5.35 (m, 0.5H).

¹³C NMR (125 MHz, CDCl₃): 320.3, 20.5, 20.9, 21.0, 25.7, 25.8, 27.8, 32.1, 32.3, 33.0, 33.6, 34.9, 36.9, 39.6, 46.2, 52.8, 64.9, 64.9, 104.3, 104.3, 123.0, 126.6, 143.2, 143.9.

Characteristic signal for 2-(2-(4-isopropylcyclopent-1-en-1-yl)ethyl)-1,3-dioxolane: ¹³C NMR (125 MHz, CDCl₃): 46.2 ppm

Characteristic signal for 2-(2-(3-isopropylcyclopent-1-en-1-yl)ethyl)-1,3-dioxolane: ¹³C NMR (125 MHz, CDCl₃): 52.8 ppm

Characteristic signals for 2-(2-(3-isopropylcyclopentylidene)ethyl)-1,3-dioxolane: 13C NMR (125 MHz, CDCl₃): 112.91, 112.95 ppm

### Example 42

### Preparation of 3-(4-isopropylcyclopent-1-en-1-yl)propanal/3-(3-isopropylcyclopent-1-en-1-yl)propanal

The compound (as a mixture) was prepared according to procedure reported in Example 3 using, as a starting material, the compound prepared in Example 41.

### 3-(4-isopropylcyclopent-1-en-1-yl)propanal/3-(3-isopropylcyclopent-1-en-1-yl)propanal ˜1/1 Mixture

¹H-NMR (500.15 MHz): 0.83, 0.86, 0.86, 0.87 (d, 6H, J=6.7 Hz), 1.43-1.54 (m, 1.5H) 1.94-2.03 (m, 2H), 2.18-2.45 (m, 4.5H), 2.54-2.60 (m, 2H), 5.28-5.31 (m, 0.5H), 5.32-5.34 (m, 0.5H), 9.75 (t, 0.5H, J=1.86 Hz), 9.77 (t, 0.5H, J=1.74 Hz).

### 3-(4-isopropylcyclopent-1-en-1-yl)propanal

¹³C NMR (150 MHz, CDCl₃): δ 20.9, 21.0, 23.9, 33.5, 36.9, 39.7, 41.8, 46.1, 124.0, 142.0, 202.6.

### 3-(3-isopropylcyclopent-1-en-1-yl)propanal

¹³C NMR (150 MHz, CDCl₃): δ 20.2, 20.5, 23.8, 27.7, 32.9, 35.0, 42.0, 52.8, 127.5, 142.7, 202.6.

### Example 43

### Hydroformylation of cis/trans-4-butyl-1-vinylcyclohexan-1-ol

The autoclave was charged with cis/trans-4-butyl-1-vinylcyclohexan-1-ol (5.01 g, 27.48 mmol, c/t=39/61), 1,2-ethanediol (6.05 g, 97.47 mmol), BiPhePhos (0.00066 mmol) and Rh(CO)₂acac (0.00021 mmol). The vessel was purged with H₂/CO 1:1 (4×5 bar), pressurized at 10 bar and then heated under vigorous stirring at 90° C. and constant pressure for 60 h to reach 96% conversion. The product mixture (10.48 g) is made up of cis/trans starting material (2.5%, 1.3%), hydrogenated starting material (0.36%), 2-((8-butyl-1-oxaspiro[4.5]decan-2-yl)oxy)ethan-1-ol (27.9%, 45.4%). 20% of 1,2-bis((8-butyl-1-oxaspiro[4.5]decan-2-yl)oxy)ethane were identified by GC/GCMS. The two diastereomeric of 2-((8-butyl-1-oxaspiro[4.5]decan-2-yl)oxy)ethan-1-ols were isolated by column chromatography on silica gel (pentane/Et₂O—1/1) and identified by spectral means.

### Cis-2-((8-butyl-1-oxaspiro[4.5]decan-2-yl)oxy)ethan-1-ol

¹H-NMR (500.15 MHz, CDCl₃): 30.88 (m, 3H), 1.13-2.17 (m, 20H), 3.62-3.80 (m, 4H), 5.09 (dd, 1H).

¹³C NMR (125 MHz, CDCl₃): δ 14.1 (q), 23.0 (t), 29.2 (t), 29.3 (t), 29.7 (t), 32.3 (t), 36.0 (t), 36.4 (t), 36.6 (d), 37.3 (t), 38.2 (t), 62.6 (t), 71.0 (t), 84.0 (s), 105.2 (d).

### Trans-2-((8-butyl-1-oxaspiro[4.5]decan-2-yl)oxy)ethan-1-ol

¹H-NMR (500.15 MHz, CDCl₃): 30.89 (m, 3H), 1.12-2.79 (m, 20H), 3.58-3.80 (m, 4H), 5.08 (dd, 1H).

¹³C NMR (125 MHz, CDCl₃): δ 14.1 (q), 23.0 (t), 29.4 (t), 30.8 (t), 31.3 (t), 32.0 (t), 32.7 (t), 36.2 (t), 36.5 (d), 36.8 (t), 39.5 (t), 62.5 (t), 70.8 (t), 86.1 (s), 104.5 (d).

### Example 44

### Preparation of 2-(2-(4-butylcyclohex-1-en-1-yl)ethyl)-1,3-dioxolane from 2-((8-butyl-1-oxaspiro[4.5]decan-2-yl)oxy)ethan-1-ol and 1,2-bis((8-butyl-1-oxaspiro[4.5]decan-2-yl)oxy)ethane

In a 100 mL reactor (equipped with a Dean-Stark apparatus and a small column, 2 elements sulzer EX) 5 g of the crude of Example 43 (hydroformylation reaction, 13.11 mmol 4-butyl-1-vinyl-cyclohexan-1-ol) was stirred with 20 mL Xylene, 8.11 g (130.6 mmol) ethylene glycol and 64.1 mg (0.65 mmol, 2.5 mol %) (98%) H₂SO₄. The mixture was heated to 130° C.-138° C. and water was collected (Dean Stark apparatus). After 4 hours 97% conversion of 2-((8-butyl-1-oxaspiro[4.5]decan-2-yl)oxy)ethan-1-ol and 1,2-bis((8-butyl-1-oxaspiro[4.5]decan-2-yl)oxy)ethane was observed by GC. After cooling down to room temperature the mixture was diluted with 20 mL of MTBE and washed with 10 mL of water. The organic phase was then washed with 10 mL of a saturated aqueous NaHCO₃ solution, once with 10 mL of water, and with 10 mL of a saturated aqueous NaCl solution. After drying over sodium sulfate the solvent was evaporated under reduced pressure (50° C., 15 mbar).

The crude (3.12 g) was purified by column chromatography (80 g cartridge, from cyclohexane to cyclohexane/MTBE 90/10 to cyclohexane/MTBE 50) 1.95 g (8.12 mmol) 2-(2-(4-butylcyclohex-1-en-1-yl)ethyl)-1,3-dioxolane were obtained (62% yield over 2 steps) and 175 mg (0.73 mmol, 6% yield over 2 steps) 2-(2-(4-butylcyclohexylidene)ethyl)-1,3-dioxolane).

### 2-(2-(4-butylcyclohex-1-en-1-yl)ethyl)-1,3-dioxolane

¹H-NMR (500.15 MHz): 0.89 (t, 3H, J=7.0 Hz), 1.14-1.34 (m, 7H), 1.39-1.40 (m, 1H), 1.55-1.65 (m, 1H), 1.70-1.79 (m, 3H), 1.89-2.12 (m, 5H), 3.82-3.89 (m, 2H), 3.92-4.01 (m, 2H), 4.85 (t, 1H, J=4.8 Hz), 5.38-5.42 (m, 1H).

¹³C NMR (125 MHz, CDCl₃): δ 14.2, 23.0, 28.5, 29.3, 29.4, 31.9, 32.1, 32.2, 33.5, 36.2, 64.9, 104.5, 120.6, 136.8.

Characteristic signals for 2-(2-(4-butylcyclohexylidene)ethyl)-1,3-dioxolane: ¹³C NMR (125 MHz, CDCl₃): 114.0 ppm.

### Example 45

### Preparation of 3-(4-butylcyclohex-1-en-1-yl)propanal

The compound was prepared according to procedure reported in Example 3 using, as a starting material, the compound prepared in Example 44. The ¹H and ¹³C-NMR analysis results in CDCl₃ were in accordance with data from literature (see R. Moretti WO 2019185599 A1).

### Example 46

### Preparation of 2-(2-(4,4-dimethylcyclohex-1-en-1-yl)ethyl)-1,3-dioxolane from 2-((8,8-dimethyl-1-oxaspiro[4.5]decan-2-yl)oxy)ethan-1-ol and 1,2-bis((8,8-dimethyl-1-oxaspiro[4.5]decan-2-yl)oxy)ethane Using Different Catalysts

### General Procedure for Testing Several Acids and Lewis Acids:

In a 100 mL reactor (equipped with a Dean-Stark apparatus and a small column, 2 elements sulzer EX) 2.0 g of the crude from Example 1 (hydroformylation reaction, 5.13 mmol 4,4-dimethyl-1-vinylcyclohexan-1-ol) was stirred with 20 mL xylene, 1.12 g (18.03 mmol) ethylene glycol and the acid or Lewis acid. The mixture was heated to reflux and water was collected (Dean Stark apparatus). After the full conversion of 2-((8,8-dimethyl-1-oxaspiro[4.5]decan-2-yl)oxy)ethan-1-ol and 1,2-bis((8,8-dimethyl-1-oxaspiro[4.5]decan-2-yl)oxy)ethane was observed by GC the mixture was cooled down to room temperature and 831 mg decane were added for the yield determination with internal standard.

Acid: 382.4 mg (5 mol %) Cu(OTf)₂ (138° C., 2 h 5 mol %, 2 h 20 mol %):

A yield of 62% of 2-(2-(4,4-dimethylcyclohex-1-en-1-yl)ethyl)-1,3-dioxolane/2-(2-(4,4-dimethylcyclohexylidene)ethyl)-1,3-dioxolane (ratio 95.6/4.4) was obtained.

Acid: 170 mg (5 mol %) Bi(OTf)₃ (1 h at 138° C.):

A yield of 77.4% of 2-(2-(4,4-dimethylcyclohex-1-en-1-yl)ethyl)-1,3-dioxolane/2-(2-(4,4-dimethylcyclohexylidene)ethyl)-1,3-dioxolane (ratio 95.5/4.5) was obtained.

Acid: 367 mg (20 mol %) SnCl₄ (at 138° C., 2 h 5 mol %, 2 h 10 mol %, 4 h 20 mol %):

A yield of 84.5% of 2-(2-(4,4-dimethylcyclohex-1-en-1-yl)ethyl)-1,3-dioxolane/2-(2-(4,4-dimethylcyclohexylidene)ethyl)-1,3-dioxolane (ratio 94.3/5.7) was obtained.

Acid: 58 mg (20 mol %) ZnBr₂ (at 138° C., 3 h):

17% of 2-(2-(4,4-dimethylcyclohex-1-en-1-yl)ethyl)-1,3-dioxolane and unreacted starting material 2-((8,8-dimethyl-1-oxaspiro[4.5]decan-2-yl)oxy)ethan-1-ol were observed by GC.

Acid: 96 mg (5 mol %) Zn(OTf)₂ (4 h at 138° C.):

A yield of 76% of 2-(2-(4,4-dimethylcyclohex-1-en-1-yl)ethyl)-1,3-dioxolane/2-(2-(4,4-dimethylcyclohexylidene)ethyl)-1,3-dioxolane (ratio 95.7/4.3) was obtained.

Acid: 200.8 mg (20 mol %) BF₃ (AcOH)₂ (4 h at 138° C.):

A yield of 67% of 2-(2-(4,4-dimethylcyclohex-1-en-1-yl)ethyl)-1,3-dioxolane/2-(2-(4,4-dimethylcyclohexylidene)ethyl)-1,3-dioxolane (ratio 94/6) was obtained.

Acid: 168 mg (20 mol %) FeCl₃ (at 138° C., 1 h 5 mol %, 2 h 20 mol %):

30% of 2-(2-(4,4-dimethylcyclohex-1-en-1-yl)ethyl)-1,3-dioxolane and unreacted starting material 2-((8,8-dimethyl-1-oxaspiro[4.5]decan-2-yl)oxy)ethan-1-ol were observed by GC.

Acid: 68 mg (2.5 mol %) Fe(OTf)₃ (at 138° C., 4 h):

A yield of 86.2% of 2-(2-(4,4-dimethylcyclohex-1-en-1-yl)ethyl)-1,3-dioxolane/2-(2-(4,4-dimethylcyclohexylidene)ethyl)-1,3-dioxolane (ratio 95.8/4.2) was obtained.

Acid: 154 mg (5 mol %) Fe(OTs)₃ (at 138° C., 8 h):

A yield of 80.1% of 2-(2-(4,4-dimethylcyclohex-1-en-1-yl)ethyl)-1,3-dioxolane/2-(2-(4,4-dimethylcyclohexylidene)ethyl)-1,3-dioxolane (ratio 95.5/4.5) was obtained.

Acid: 19.4 mg (2.5 mol %) TfOH (at 138° C., 5 h):

A yield of 90.0% of 2-(2-(4,4-dimethylcyclohex-1-en-1-yl)ethyl)-1,3-dioxolane/2-(2-(4,4-dimethylcyclohexylidene)ethyl)-1,3-dioxolane (ratio 94.7/5.3) was obtained.

Acid: 80 mg Amberlyst 15 dry (at 138° C., 4 h):

A yield of 85.4% of 2-(2-(4,4-dimethylcyclohex-1-en-1-yl)ethyl)-1,3-dioxolane/2-(2-(4,4-dimethylcyclohexylidene)ethyl)-1,3-dioxolane (ratio 94.5/5.5) was obtained (conversion 96%).

### Example 47

### Hydroformylation of 1-(1-butoxyethoxy)-4,4-dimethyl-1-vinyl-cyclo-hexane

The autoclave was charged with 1-(1-butoxyethoxy)-4,4-dimethyl-1-vinyl-cyclo-hexane (5.01 g, 19.693 mmol, 1,2-ethanediol (3.84 g, 61.866 mmol), BiPhePhos solution (5.9×10-mmol) and Rh(CO)₂acac (2×10⁻⁵ mmol [Rh]). The vessel was purged with H₂/CO 1:1 (4×5 bar) and heated under vigorous stirring at 90° C. and 10 bar syngas pressure for 48 h. After cooling and depressurization, the crude was diluted with water (15 ml) and extracted with Et₂O (2×30 ml). The organic layers were combined, dried over anhydrous sodium sulfate, filtered and concentrated to give a residue (4.66 g) containing 2-((8,8-dimethyl-1-oxaspiro[4.5]decan-2-yl)oxy)ethan-1-ol (22.9%), 1,2-bis((8,8-dimethyl-1-oxaspiro[4.5]decan-2-yl)oxy)ethane (4%), 2-((1,3-dioxolan-2-yl)methoxy)-8,8-dimethyl-1-oxaspiro[4.5]decane (2.2%), 2-((4,4-dimethyl-1-vinylcyclohexyl)oxy)ethan-1-ol (1.4%), 2-butoxy-8,8-dimethyl-1-oxaspiro[4.5]decane (39.4%) and some minor unidentified products. The products were isolated by chromatography (Et₂O/pentane—1/1) and identified by spectral means.

### 2-butoxy-8,8-dimethyl-1-oxaspiro[4.5]decane

¹H-NMR (500.15 MHz, CDCl₃): δ 0.90 (s, 3H), 0.91 (t, 3H), 0.94 (s, 3H), 1.17-2.03 (m, 16H), 3.34 (m, 1H), 3.68 (m, 1H), 5.05 (m, 1H).

¹³C NMR (125 MHz, CDCl₃): 313.9 (q), 19.5 (t), 28.1 (q), 28.2 (q), 29.4 (s), 31.9 (t), 32.5 (t), 33.8 (t), 35.5 (t), 36.7 (t), 36.8 (t), 66.6 (t), 84.1 (s), 103.5 (d).

The mixture obtained could be transformed to 2-(2-(4,4-dimethylcyclohex-1-en-1-yl)ethyl)-1,3-dioxolane (81% yield) in the presence of xylene, sulfuric acid (2.5 mol %) and 5 eq 1,2-ethanediol (azeotropic distillation of water).

### Example 48

### Hydroformylation of 3,7-Dimethyl-1-octen-3-ol

The autoclave was charged with 3,7-dimethyl-1-octen-3-ol (5.43 g, 34.19 mmol), 1,2-ethanediol (6.53 g, 105.2 mmol), BiPhePhos (3×10⁻⁶ mmol) and Rh(CO)₂acac (10⁻⁶ mmol [Rh]). The vessel was purged with H₂/CO (1:1, 4×5 bar), pressurized at 10 bar and then heated under vigorous stirring at 90° C. and constant pressure for 48 h to reach 94% conversion. The crude was diluted with water (15 mL), extracted with Et₂O (2×30 mL) and the combined organic layers dried with anhydrous sodium sulfate. The mixture was filtered and concentrated to give a transparent liquid residue (7.21 g). GLC analysis of the residue revealed the presence of starting 3,7-dimethyl-1-octen-3-ol (5.9%), 2-((5-methyl-5-(4-methylpentyl)tetrahydrofuran-2-yl)oxy) ethan-1-ol (32.6%, 35.3%, 62% yield for both distereomers), 5,5′-oxybis(2-methyl-2-(4-methylpentyl)tetrahydrofuran) (1.6%, 2% yield), 1,2-bis((5-methyl-5-(4-methylpentyl)tetrahydrofuran-2-yl)oxy)ethane (17.6%, 19% yield) and 3,7-dimethyloctan-3-ol (2.9%). The products were isolated by chromatography (Et₂O/pentane—1:1) and identified by NMR.

### 2-((5-methyl-5-(4-methylpentyl)tetrahydrofuran-2-yl)oxy) ethan-1-ol (Diast.Mix)

¹H-NMR (500.15 MHz, CDCl₃): δ 0.84-0.89 (m, 6H), 1.11-1.33 (m, 4H), 1.34 (s, 3H), 1.36-2.02 (m, 6H), 2.03-2.15 (m, 1H), 2.74 (s, 1H), 3.62-3.77 (m, 4H), 5.07 (m, 1H).

¹³C NMR (125 MHz, CDCl₃): δ 22.3 (t), 22.6 (q), 22.6 (q), 27.7 (q), 27.9 (d), 33.2 (t), 34.8 (t), 39.4 (t), 42.0 (t), 62.6 (t), 71.0 (t), 85.3 (s), 105.4 (d).

¹³C NMR (125 MHz, CDCl₃): δ 22.6 (q), 22.6 (q), 22.8 (t), 25.8 (q), 27.9 (d), 32.7 (t), 34.8 (t), 39.4 (t), 43.1 (t), 62.4 (t), 70.6 (t), 85.6 (s), 105.0 (d).

### 1,2-bis((5-methyl-5-(4-methylpentyl)tetrahydrofuran-2-yl)oxy)ethane (Diast.Mix)

¹H-NMR (500.15 MHz, CDCl₃): δ 0.82-0.90 (m, 12H), 1.09-2.08 (m, 28H), 3.46-3.60 (m, 2H), 3.73-3.86 (m, 2H), 25.3-5.1 (m, 2H).

¹³C NMR (125 MHz, CDCl₃): 322.39 (t), 22.55-22.7 (q), 22.7-22.81(t), 26.0-26.14 (q), 27.9-28.0 (d), 28.23-28.34 (q), 32.6-32.76 (t), 33.27-33.40 (t), 34.24-34.38 (t), 34.27-34.86 (t), 39.48-39.64 (t), 42.27-42.38 (t), 43.13-43.25 (t), 65.4-66.1 (t), 84.67-85.0 (s), 103.65-104.25 (d).

### Example 49

### Preparation of unsaturated dioxolanes from 2-((5-methyl-5-(4-methylpentyl)tetrahydrofuran-2-yl)oxy)ethan-1-ol and 1,2-bis((5-methyl-5-(4-methylpentyl)tetrahydrofuran-2-yl)oxy)ethane

In a 10 mL reactor (equipped with a Dean-Stark apparatus and a small column, 2 elements sulzer EX) 1.9 g (8.249 mmol) of 2-((5-methyl-5-(4-methylpentyl)tetrahydrofuran-2-yl)oxy)ethan-1-ol from Example 48 was stirred with 5 mL Xylene, 4.10 g (65.99 mmol, 8 eq) 1,2-ethanediol and 18.4 mg (0.188 mmol, 2.3 mol %) (98%) H₂SO₄. The mixture was heated to 130° C.-138° C.) and water was collected (Dean Stark apparatus). After 3 hours a full conversion of 2-((5-methyl-5-(4-methylpentyl)tetrahydrofuran-2-yl)oxy)ethan-1-ol was observed by GC. The mixture is diluted with 10 mL of MTBE and washed with 5 mL of water. The organic phase was then washed with 5 mL of a saturated aqueous NaHCO₃ solution, once with 5 mL of water, and with 5 mL of a saturated aqueous NaCl solution. After drying over sodium sulfate the solvent was evaporated under reduced pressure (45° C., 15 mbar). The crude (2.06 g) was purified by column chromatography (80 g cartridge, from cyclohexane 100% to MTBE 100%). 1568 mg (7.385 mmol, 89.5% yield) of a mixture of isomers (ratio 32/18/27/16/4) was isolated ((E)-2-(3,7-dimethyloct-3-en-1-yl)-1,3-dioxolane/(Z)-2-(3,7-dimethyloct-3-en-1-yl)-1,3-dioxolane/(E)-2-(3,7-dimethyloct-2-en-1-yl)-1,3-dioxolane/(Z)-2-(3,7-dimethyloct-2-en-1-yl)-1,3-dioxolane/2-(7-methyl-3-methyleneoctyl)-1,3-dioxolane, ratio 32/18/27/16/4).

### (E)-2-(3,7-dimethyloct-3-en-1-yl)-1,3-dioxolane (Characteristic Signals)

¹³C NMR (125 MHz, CDCl₃): 3125.1, 133.7.

GC 3.20 min (column DB-1 10 m, 80° C.-320° C. (30° C./min)

### (Z)-2-(3,7-dimethyloct-3-en-1-yl)-1,3-dioxolane (Characteristic Signals)

¹³C NMR (125 MHz, CDCl₃): 3126.2, 133.9.

GC 3.04 min (column DB-1 10 m, 80° C.-320° C. (30° C./min)

### (E)-2-(3,7-dimethyloct-2-en-1-yl)-1,3-dioxolane (Characteristic Signals)

¹³C NMR (125 MHz, CDCl₃): 3117.0, 139.0.

GC 3.16 min (column DB-1 10 m, 80° C.-320° C. (30° C./min)

### (Z)-2-(3,7-dimethyloct-2-en-1-yl)-1,3-dioxolane (Characteristic Signals)

¹³C NMR (125 MHz, CDCl₃): 3117.7, 139.2.

GC 3.10 min (column DB-1 10 m, 80° C.-320° C. (30° C./min)

### 2-(7-methyl-3-methyleneoctyl)-1,3-dioxolane (Characteristic Signals)

¹³C NMR (125 MHz, CDCl₃): 3108.8, 149.2.

GC 3.13 min (column DB-1 10 m, 80° C.-320° C. (30° C./min)

(E)-2-(3,7-dimethyloct-3-en-1-yl)-1,3-dioxolane/(Z)-2-(3,7-dimethyloct-3-en-1-yl)-1,3-dioxolane/(E)-2-(3,7-dimethyloct-2-en-1-yl)-1,3-dioxolane/(Z)-2-(3,7-dimethyloct-2-en-1-yl)-1,3-dioxolane/2-(7-methyl-3-methyleneoctyl)-1,3-dioxolane could be deprotected to the unsaturated aldehydes according the procedure of Example 3 (99% yield on conversion).

¹³C NMR (125 MHz, CDCl₃) major isomer (E)-4,8-dimethylnon-4-enal: δ 16.0, 22.5, 25.8, 27.6, 31.9, 38.9, 42.2, 126.1, 132.5, 202.8.

### (E)-4,8-dimethylnon-4-enal/(E)-4,8-dimethylnon-3-enal/(Z)-4,8-dimethylnon-4-enal/(Z)-4,8-dimethylnon-3-enal/8-methyl-4-methylenenonanal

¹³C NMR (125 MHz, CDCl₃) characteristic signals of the mixture:

δ 109.5, 112.5, 113.2, 126.1, 127.2, 132.5, 132.5, 141.8, 141.9, 147.9.

1. A process for preparing a compound of formula 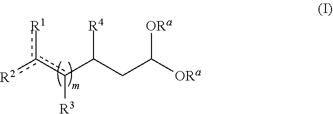
in the form of any one of its stereoisomers or a mixture thereof, wherein one dotted line is a carbon-carbon double bond and the others are carbon-carbon single bonds; m is 0 or 1; R¹ represents a C₁₋₁₂ hydrocarbon group, optionally comprising one to three oxygen atoms and/or one to two nitrogen atoms and/or one sulfur atom; R² represents a hydrogen atom or a R¹ group; or R¹ and R² are taken together and form a C₅₋₁₈ cycloalkyl or C₅₋₁₈ cycloalkenyl group, each optionally substituted by one or more hydroxy, C₁₋₁₅ alkyl, C₂₋₁₅ alkenyl, C₁₋₁₅ alkoxy, C₃₋₁₅ cycloalkyl, C₅₋₁₅ cycloalkenyl, C₁₋₆ ester, COOH, C₆₋₁₀ aryl and/or C₆₋₁₀ aryloxy groups; R³ and R⁴, independently from each other, represent a hydrogen atom or a C₁₋₆ alkyl group; both R^{a} are taken together and represent a C₂₋₂₀ hydrocarbylene group, optionally comprising one to five oxygen atoms;
the process comprising reacting a compound of formula (II) 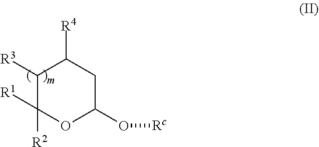
in the form of any one of its stereoisomers or a mixture thereof, wherein R¹, R², R³, R⁴ and m have the same meaning as defined in formula (I) and R^{c} is a hydrogen atom, C₁₋₁₂ hydrocarbon group optionally comprising one to five oxygen atoms or R^{c} is a group of formula 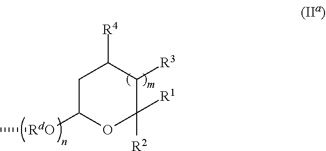
wherein R¹, R², R³, R⁴ and m have the same meaning as defined in formula (I); n is 0 or 1, R^{d} is a C₁₋₁₂ hydrocarbylene group optionally comprising one to five oxygen atoms and the hatched line indicates the location of the bond between (II^{a}) and the oxygen atom of formula (II);
with an acid and a polyol or an alpha hydroxy carboxylic acid.

2. The process according to claim 1, wherein R³ and R⁴, independently from each other, represent a hydrogen atom or a C₁₋₃ alkyl group.

3. The process according to claim 1, wherein m is 0.

4. The process according to claim 1, wherein R¹ and R² are taken together and form C₅₋₁₂ cycloalkyl or C₅₋₁₂ cycloalkenyl group, each optionally substituted by one or two C₁₋₁₀ alkyl groups.

5. The process according to claim 1, wherein R^{c} is C₁₋₇ alkyl group optionally substituted by one oxo group or one or two hydroxy groups.

6. The process according to claim 1, wherein the compound of formula (I) is of formula 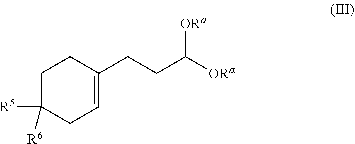
in the form of any one of its stereoisomers or a mixture thereof, wherein each R^{a} has the same meaning as defined in claim 1 and R⁵ and R⁶, independently from each other, represent a hydrogen atom or a C₁₋₆ alkyl group;
and said compound of formula (II) is of formula 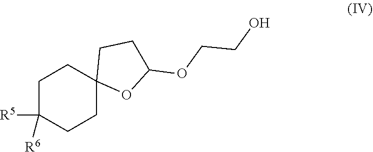
in the form of any one of its stereoisomers or a mixture thereof, and wherein each R⁵ and R⁶ have the same meaning as defined in formula (III).

7. The process according to claim 1, wherein the acid has a pH below 3.

8. The process according to claim 1, wherein the polyol is of formula HOROH wherein R is a C₂₋₆ alkanediyl group optionally substituted by one or two hydroxy groups and the alpha hydroxy carboxylic acid is of formula R′CH(OH)COOH wherein R′ is a hydrogen atom or a C₁₋₆ alkyl group.

9. The process according to claim 1, further comprising the step of converting compound of formula (I) to compound of formula 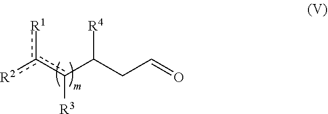
in the form of any one of its stereoisomers or a mixture thereof, and wherein the dotted lines, m, R¹, R², R³ and R⁴ have the same meaning as defined in claim 1.

10. A process for preparing a compound of formula 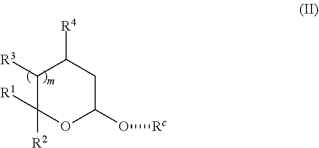
in the form of any one of its stereoisomers or a mixture thereof, wherein m is 0 or 1; each R¹ represents a C₁₋₁₂ hydrocarbon group, optionally comprising one to three oxygen atoms and/or one to two nitrogen atoms and/or one sulfur atom; R² represents a hydrogen atom or a R¹ group; or R¹ and R² are taken together and form C₅₋₁₈ cycloalkyl or C₅₋₁₈ cycloalkenyl group, each optionally substituted by one or more hydroxy, C₁₋₁₅ alkyl, C₂₋₁₅ alkenyl, C₁₋₁₅ alkoxy, C₃₋₁₅ cycloalkyl, C₅₋₁₅ cycloalkenyl, C₁₋₆ ester, COOH, C₆₋₁₀ aryl and/or C₆₋₁₀ aryloxy groups; R³ and R⁴, independently from each other, represent a hydrogen atom or a C₁₋₆ alkyl group; and R^{c} is a is a hydrogen atom, a C₁₋₁₂ hydrocarbon group optionally comprising one to five oxygen atoms or R^{c} is a group of formula 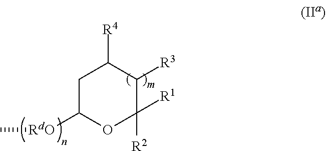
wherein R¹, R², R³, R⁴ and m have the same meaning as defined in formula (II); n is 0 or 1, R^{d} is a C₁₋₁₂ hydrocarbylene group optionally comprising one to five oxygen atoms and the hatched line indicates the location of the bond between (II^{a}) and the oxygen atom of formula (II);
by the hydroformylation of a compound of formula 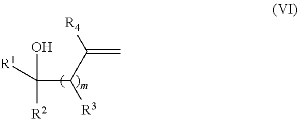
in the form of any one of its stereoisomers or a mixture thereof, and wherein m, R¹, R²,
R³ and R⁴ have the same meaning as defined in formula (II);
in a presence of a hydroformylation catalyst comprising a bidentate phosphorous ligand and an alcohol or a polyol.

11. The process according to claim 10, wherein the hydroformylation catalyst is a Rhodium catalyst.

12. The process according to claim 10, wherein the alcohol or polyol is added into the reaction medium of the process at a concentration of at least 1 equivalent, relative to the amount of the compound of formula (VI).

13. The process according to claim 10, wherein the bidentate phosphorous ligand is a bidentate phosphite ligand.

14. The process according to claim 10, wherein the polyol is a diol.

15. A compound of formula 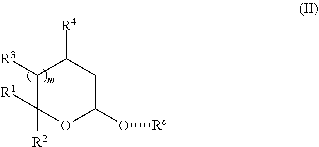
in the form of any one of its stereoisomers or a mixture thereof, wherein m is 0 or 1; R¹ and R², independently from each other, represent a C₁₋₁₂ hydrocarbon group, optionally comprising one to three oxygen atoms and/or one to two nitrogen atoms and/or one sulfur atom; or R¹ and R² are taken together and form C₅₋₁₈ cycloalkyl or C₅₋₁₈ cycloalkenyl group, each optionally substituted by one or more hydroxy, C₁₋₁₅ alkyl, C₂₋₁₅ alkenyl, C₁₋₁₅ alkoxy, C₃₋₁₅ cycloalkyl, C₅₋₁₅ cycloalkenyl, C₁₋₆ ester, COOH, C₆₋₁₀ aryl and/or C₆₋₁₀ aryloxy groups; R³ and R⁴, independently from each other, represent a hydrogen atom or a C₁₋₆ alkyl group; and R^{c} is a hydrogen atom, a C₁₋₁₂ hydrocarbon group optionally comprising one to five oxygen atoms or a R^{c} is a group of formula 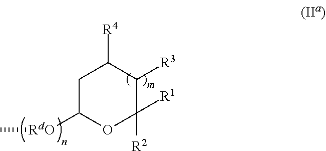
wherein R¹, R², R³, R⁴ and m have the same meaning as defined in formula (II); n is 0 or 1, R^{d} is a C₁₋₁₂ hydrocarbylene group optionally comprising one to five oxygen atoms and the hatched line indicates the location of the bond between (II^{a}) and the oxygen atom of formula (II); provided that 4-butyl-1-[(5,5-dibutyltetrahydro-2-furayl)oxy]-2,4-octanediol and 2-[(Tetrahydro-5,5-dimethyl-2-furanyl)oxy]ethanol are excluded.

16. The process according to claim 1, wherein R³ and R⁴, independently from each other, represent a hydrogen atom.

17. The process according to claim 1, wherein R^{c} is a (CH₂)ₙOH group wherein n is 2 or 3 or 4.

18. The process according to claim 1, wherein the acid is selected from the group consisting of pTsOH, MsOH, TfOH, H₂SO₄, HCl, H₃PO₄, KHSO₄, NaHSO₄, BF₃·Et₂O, BF₃·(AcOH)₂, ZnBr₂, Fe(OTf)₃, Fe(OTs)₃, FeCl₃, Zn(OTf)₂, Bi(OTf)₃, Cu(OTf)₂, SnCl₄, Alox acidic, Amberlyst 15, Amberlyst 35, SiO₂, Wayphos, polyphosphoric acid, Zeolite, boric acid, CSA, Bentonite, and Clay.

19. The process according to claim 10, wherein the alcohol or polyol is added into the reaction medium of the process at a concentration of at least 3.4 equivalents, relative to the amount of the compound of formula (VI).
